(19) United States
(12) Patent Application Publication
(10) Pub. No.: US 2007/0292880 A1
(43) Pub. Date: Dec. 20, 2007

(54) Compositions and methods for detecting predisposition to a substance use disorder or to a mental illness or syndrome

(76) Inventors: Robert Philibert, University Heights, IA (US); Anup Madan, Bellevue, WA (US)

Correspondence Address: VIKSNINS HARRIS & PADYS PLLP, P.O. BOX 111098, ST. PAUL, MN, 55111-1098, US

(21) Appl. No.: 11/800,629

(22) Filed: May 7, 2007

Related U.S. Application Data
(60) Provisional application No. 60/798,259, filed on May 5, 2006.

(51) Int. Cl. C12Q 1/68; G01N 33/00

(52) U.S. Cl. 435/6; 436/94

(57) ABSTRACT

The present invention provides screening kits, compositions, and diagnostic methods for determining whether a subject has a predisposition to, or likelihood of having, a substance use disorder or mental illness or syndrome by determining a nucleic acid expression profile from a biological sample from the subject, wherein a given profile indicates that the subject has a predisposition to a substance use disorder or mental illness or syndrome.

### RELATED APPLICATION

This application claims priority under 35 U.S.C. 119(e) to provisional application U.S. Ser. No. 60/798,259, filed May 5, 2006, which application is incorporated hereby by reference.

### STATEMENT OF GOVERNMENT SUPPORT

Work related to this invention was funded by the U.S. government (NIH Grants K08MH064714, R01DA015789 and R01AI053264). The government has certain rights in this patent.

### BACKGROUND

Substance use disorders and mental illnesses and syndromes cause serious problems, both for the affected individuals and for society in general. Despite intensive research, however, a reliable laboratory test for diagnosing a patient as having, or for being at risk for developing, such conditions has not been developed. Such diagnoses are still generally made clinically, on the basis of observed behavior. Given the difficulties of defining normal experience and behavior and the lack of reliable objective indicators, it is not surprising that to date that the systems of diagnosis in psychiatry have been less than satisfactory. A reliable laboratory test would be of practical value in everyday clinical practice, for example, in assisting doctors in prescribing the appropriate treatment for their patients. Thus, methods of identifying subjects that have, or are at risk for developing, substance use disorders or mental illnesses or syndromes are needed.

### SUMMARY OF CERTAIN EMBODIMENTS OF THE INVENTION

The present invention provides a screening kit for determining whether a subject has a predisposition to, or likelihood of having, a substance use disorder or mental illness or syndrome. The screening kit includes the following: (a) a solid substrate, at least one probe specific for a down-regulated gene associated with nicotine dependence, and/or at least one probe specific for an up-regulated gene associated with nicotine dependence, wherein each probe is bound onto the substrate in a distinct spot; (b) a solid substrate, at least one probe specific for a down-regulated gene associated with panic disorder, and/or at least one probe specific for an up-regulated gene associated with panic disorder, wherein each probe is bound onto the substrate in a distinct spot; (c) a solid substrate, at least one probe specific for a down-regulated gene associated with Antisocial Personality Disorder (ASPD), and/or at least one probe specific for an up-regulated gene associated ASPD, wherein each probe is bound on the substrate in a distinct spot; (d) a solid substrate, at least one probe specific for a down-regulated gene associated with Major Depressive Disorder (MDD), and/or at least one probe specific for an up-regulated gene associated with MDD, wherein each probe is bound onto the substrate in a distinct spot; and (e) a solid substrate, at least one probe specific for a down-regulated gene associated with positive symptom schizophrenia such as that associated with the HOPA^{12bp} allele, and/or at least one probe specific for an up-regulated gene associated with positive symptom schizophrenia such as that associated with the HOPA^{12bp} allele, wherein each probe is bound onto the substrate in a distinct spot. In certain embodiments, the probe is an oligonucleotide probe. In certain embodiments, the probe is a nucleic acid derivative probe. The probes may be labeled.

In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. The solid substrate may contain only probes in a certain category (e.g., probes specific for down-regulated genes associated with nicotine dependence), or may containing probes of more than one category (e.g., probes specific for down-regulated genes associated with nicotine dependence and probes specific for down-regulated genes associated with panic disorder). In one kit, all of the probes may be physically located on a single solid substrate or on multiple substrates. A single category of probes may be located on a single solid substrate, or on multiple substrates. In a certain embodiment, the solid substrate may contain about 40-45 down-regulated probes and about 40-45 up-regulated probes.

In certain embodiments, the current invention can also take the form of a PCR (polymerize chain reaction) assay. In some cases, this will take the form of real time PCR assays (RTPCR) assays. In certain embodiments of these PCR assays, a kit may contain two primers that specifically amplify a region of a gene transcript and gene specific probe that selectively recognizes the amplified region. Together, the primers and the gene specific probes are referred to as a primer-probe set. By measuring the amount of gene specific probe that has hybridized to an amplified segment at a given point of the PCR reaction or throughout the PCR reaction, one who is skilled in the art can infer the amount of transcript originally present at the start of the reaction. In some cases, the amount of probe hybridized is measured through fluorescence spectrophotometry. The number of primer-probe sets can be any integer between 1 and 10,000 probes, such as 1, 2, 3, 4, 5, 6, 7, 8, 9, 10, . . . 9997, 9998, 9999, 10,000. The PCR kit may contain only primer-probe sets in a certain category (e.g., specific for down-regulated genes associated with nicotine dependence), or may containing primer-probe sets of more than one category (e.g., specific for down-regulated genes associated with nicotine dependence and oligonucleotide probes specific for down-regulated genes associated with panic disorder). In one kit, all of the probes may be physically located in a single reaction well or in multiple reaction wells. A single category of probes may be in dry or in liquid form. They may be used in a single reaction or in a series of reactions. In a certain embodiments, the PCR kit may contain about 40-45 down-regulated primer-probe sets and about 40-45 up-regulated primer-probe sets. In certain embodiments, the probe is an oligonucleotide probe. In certain embodiments, the probe is a nucleic acid derivative probe.

The term “substrate” refers to any solid support to which the probes may be attached. The substrate material may be modified, covalently or otherwise, with coatings or functional groups to facilitate binding of probes. Suitable substrate materials include polymers, glasses, semiconductors, papers, metals, gels and hydrogels among others. Substrates may have any physical shape or size, e.g., plates, strips, or microparticles.

The term “spot” refers to a distinct location on a substrate to which probes of known sequence or sequences are attached. A spot may be an area on a planar substrate, or it may be, for example, a microparticle distinguishable from other microparticles.

The term “bound” means affixed to the solid substrate. A spot is “bound” to the solid substrate when it is affixed in a particular location on the-substrate for purposes of the screening assay.

In certain embodiments of the kit of the present invention, the substrate is a polymer, glass, semiconductor, paper, metal, gel or hydrogel. In certain embodiments of the present invention, the kit further includes a solid substrate and at least one control probe, wherein the at least one control probe is bound onto the substrate in a distinct spot.

In certain embodiments of the present invention, the solid substrate is a microarray. An “array” or “microarray” is used synonymously herein to refer to a plurality of probes attached to one or more distinguishable spots on a substrate. A microarray may comprise a single substrate or a plurality of substrates, for example a plurality of beads or microspheres. A “copy” of a microarray contains the same types and arrangements of probes.

The present invention also provides a composition for determining whether a subject has a predisposition to, or likelihood of having, a substance use disorder or mental illness or syndrome, by determining a nucleic acid expression profile from a single type of blood cell or blood cell derivative from the subject, the method including obtaining a profile associated with the sample, wherein the profile includes quantitative data for at least one down-regulated gene associated with nicotine dependence, at least one up-regulated gene associated with nicotine dependence, at least one down-regulated gene associated with panic disorder, at least one up-regulated gene associated with panic disorder, at least one down-regulated gene associated with ASPD, at least one up-regulated gene associated with ASPD, at least one down-regulated gene associated with MDD, at least one up-regulated gene associated with MDD, at least one down-regulated gene associated with positive symptom schizophrenia such as that associated with the HOPA^{12bp} allele, or at least one up-regulated gene associated with positive symptom schizophrenia such as that associated with the HOPA^{12bp} allele; (b) inputting the data into an analytical process that uses the data to classify the sample, wherein the classification is a “substance use disorder or mental illness or syndrome” classification or a “healthy” classification; and (c) classifying the sample according to the output of the process. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

As used herein, the term “healthy” means that a subject does not manifest a particular condition, and is no more likely that at random to be susceptible to a particular condition.

The present invention also provides a composition for determining whether a subject has a predisposition to, or likelihood of having nicotine dependence including (a) a solid substrate; (b) at least one down-regulated probe specific for a down-regulated gene associated with nicotine dependence, wherein each down-regulated probe is bound onto the substrate in a distinct spot, and/or at least one up-regulated probe specific for an up-regulated gene associated with nicotine dependence, wherein each up-regulated probe is bound onto the substrate in a distinct spot. As used herein, the term “down-regulated gene associated with nicotine dependence” is defined as a probe listed in FIG. 4A, and wherein an “up-regulated gene associated with nicotine dependence” is defined as a probe listed in FIG. 4B. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

The present invention also provides a composition for determining whether a subject has a predisposition to, or likelihood of having panic disorder including (a) a solid substrate; (b) at least one down-regulated probe specific for a down-regulated gene associated with panic disorder, wherein each down-regulated probe is bound onto the substrate in a distinct spot, and/or at least one up-regulated probe specific for an up-regulated gene associated with panic disorder, wherein each up-regulated probe is bound onto the substrate in a distinct spot. As used herein, the term “down-regulated gene associated with panic disorder” is defined as a probe listed in FIG. 5A, and wherein an “up-regulated gene associated with panic disorder” is defined as a probe listed in FIG. 5B. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

The present invention also provides a composition for determining whether a subject has a predisposition to, or likelihood of having ASPD including (a) a solid substrate; (b) at least one down-regulated oligonucleotide probe specific for a down-regulated gene associated with ASPD, wherein each down-regulated probe is bound onto the substrate in a distinct spot, and/or at least one up-regulated probes specific for an up-regulated gene associated with ASPD, wherein each up-regulated probe is bound onto the substrate in a distinct spot. As used herein, the term “down-regulated gene associated with ASPD” is defined as a probe listed in FIG. 6A, and wherein an “up-regulated gene associated with ASPD” is defined as a probe listed in FIG. 6B. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

The present invention also provides a composition for determining whether a subject has a predisposition to, or likelihood of having MDD including (a) a solid substrate; (b) at least one down-regulated oligonucleotide probe specific for a down-regulated gene associated with MDD, wherein each down-regulated probe is bound onto the substrate in a distinct spot, and/or at least one up-regulated probe specific for an up-regulated gene associated with MDD, wherein each up-regulated probe is bound onto the substrate in a distinct spot. As used herein, the term “down-regulated gene associated with MDD” is defined as a probe listed in FIG. 7A, and wherein an “up-regulated gene associated with MDD” is defined as a probe listed in FIG. 7B. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

The present invention also provides a composition for determining whether a subject has a predisposition to, or likelihood of having positive symptom schizophrenia such as that associated with the HOPA^{12bp} allele including (a) a solid substrate; (b) at least one down-regulated probe specific for a down-regulated gene associated with positive symptom schizophrenia such as that associated with the HOPA^{12bp} allele, wherein each down-regulated probe is bound onto the substrate in a distinct spot, and/or at least one up-regulated probe specific for an up-regulated gene associated with positive symptom schizophrenia such as that associated with the HOPA^{12bp} allele, wherein each up-regulated probe is bound onto the substrate in a distinct spot. As used herein, the term “down-regulated gene associated with positive symptom schizophrenia such as that associated with the HOPA^{12bp} allele” is defined as a probe listed in FIG. 8A, and wherein an “up-regulated gene associated with positive symptom schizophrenia such as that associated with the HOPA^{12bp} allele” is defined as a probe listed in FIG. 8B. In certain embodiments, a solid substrate may contain a number of probes that is any integer between 1 and 10,000 probes, such as 1, 2, 3, 4, 5, 6, 7, 8, 9, 10, . . . 9997, 9998, 9999, 10,000. Each probe is specific for a different gene.

In addition to the specific biomarker sequences identified in this application by name, accession number, or sequence, the invention also contemplates use of biomarker variants that are at least 90% or at least 95% or at least 97% identical to the exemplified sequences and that are now known or later discover and that have utility for the methods of the invention. These variants may represent polymorphisms, splice variants, mutations, and the like. Various techniques and reagents find use in the diagnostic methods of the present invention. In one embodiment of the invention, blood samples, or samples derived from blood, e.g. plasma, circulating, etc. are assayed for the presence of polypeptides. Typically a blood sample is drawn, and a derivative product, such as plasma or serum, is tested. Such polypeptides may be detected through specific binding members. The use of antibodies for this purpose is of particular interest. Various formats find use for such assays, including antibody arrays; ELISA and RIA formats; binding of labeled antibodies in suspension/solution and detection by flow cytometry, mass spectroscopy, and the like. Detection may utilize one or a panel of antibodies, preferably a panel of antibodies in an array format. Expression signatures typically utilize a detection method coupled with analysis of the results to determine if there is a statistically significant match with a disease signature.

The present invention also provides a composition for determining whether a subject has a predisposition to, or likelihood of having nicotine dependence including a PCR or RTPCR assay kit comprising at least one primer-probe set specific for a down-regulated gene associated with nicotine dependence, and/or at least one primer-probe set specific for an up-regulated gene associated with nicotine dependence.

The present invention also provides a composition for determining whether a subject has a predisposition to, or likelihood of having panic disorder including a PCR or RTPCR assay kit comprising at least one primer-probe set specific for a down-regulated gene associated with panic disorder, and/or at least one primer-probe set specific for an up-regulated gene associated with panic disorder.

The present invention also provides a composition for determining whether a subject has a predisposition to, or likelihood of having Antisocial Personality Disorder (ASPD) comprising a PCR or RTPCR assay kit including at least one primer-probe set specific for a down-regulated gene associated with ASPD, and/or at least one primer-probe set specific for an up-regulated gene associated with ASPD.

The present invention also provides a composition for determining whether a subject has a predisposition to, or likelihood of having Major Depressive Disorder (MDD) including a PCR or RTPCR assay kit comprising at least one primer-probe set specific for a down-regulated gene associated with MDD, and/or at least one primer-probe set specific for an up-regulated gene associated with MDD.

The present invention also provides a composition for determining whether a subject has a predisposition to, or likelihood of having positive symptom schizophrenia such as that associated with the HOPA^{12bp} allele including a PCR or RTPCR assay kit comprising at least one primer-probe set specific for a down-regulated gene associated with positive symptom schizophrenia such as that associated with the HOPA^{12bp} allele, and/or at least one primer-probe set specific for an up-regulated gene associated with positive symptom schizophrenia such as that associated with the HOPA^{12bp} allele.

The present invention also provides a diagnostic method for determining whether a subject has a predisposition to, or likelihood of having, a substance use disorder or mental illness or syndrome, by determining a nucleic acid expression profile from a single type of blood cell or a blood cell derivative from the subject, the method involves (a) obtaining a profile associated with the sample, wherein the profile comprises quantitative data for at least one down-regulated gene associated with nicotine dependence, at least one up-regulated gene associated with nicotine dependence, at least one down-regulated gene associated with panic disorder, at least one up-regulated gene associated with panic disorder, at least one down-regulated gene associated with ASPD, at least one up-regulated gene associated with ASPD, at least one down-regulated gene associated with MDD, at least one up-regulated gene associated with MDD, at least one down-regulated gene associated with positive symptom schizophrenia such as that associated with the HOPA^{12bp} allele, or at least one up-regulated gene associated with positive symptom schizophrenia such as that associated with the HOPA^{12bp} allele; (b) inputting the data into an analytical process that uses the data to classify the sample, wherein the classification is a “substance use disorder or mental illness or syndrome” classification or a “healthy” classification; and (c) classifying the sample according to the output of the process. In certain embodiments, the analytical process comprises comparing the obtained profile with a pre-determined reference profile. In certain embodiments the reference profile comprises data obtained from one or more healthy control subjects, or comprises data obtained from one or more subjects diagnosed with a substance use disorder or mental illness or syndrome. In certain embodiments, the method further involves obtaining a statistical measure of a similarity of the obtained profile to the reference profile.

In certain embodiments the blood cell is a lymphocyte. In certain embodiments the lymphocyte type is a B-lymphocyte. In certain embodiments, the B-lymphocytes have been immortalized. In certain embodiments, the blood cell type is a monocyte. In certain embodiments, the blood cell type is a basophil.

In certain embodiments the mental illness or syndrome is major depression, antisocial personality disorder, panic disorder, bipolar disorder or schizophrenia. In certain embodiments the substance use disorder is nicotine dependence, alcohol dependence or cannabis dependence.

The present invention provides a diagnostic method for determining whether a subject has a predisposition to, or likelihood of having, a substance use disorder or a mental illness or syndrome. As used herein the term “predisposition” is defined as a tendency or susceptibility to manifest a condition. A subject is more likely than a control subject to manifest the condition. The term “substance use disorder” includes both abuse and dependence on a substance. The method involves determining a nucleic acid expression profile from cells in a biological sample from the subject, wherein a given profile indicates that the subject has a predisposition to, or likelihood of having, a mental illness or substance use disorder. The mental syndrome or illness to be diagnosed may be major depression, antisocial personality disorder, panic disorder, bipolar disorder and/or schizophrenia. The substance use disorder to be diagnosed may include nicotine dependence, alcohol dependence, and/or cannabis dependence. In certain embodiments, the nucleic acid expression profile determines the up- or down-regulation of one or more genes. In certain embodiments, the expression profile determines the up- or down-regulation of at least 1, 2, 3, 4, 5, 6, 7, 8, 9, 10, 11, 12, 13, 14, 15, 16, 17, 18, 19, 20, 21 or 22 or more genes, where each probe is specific for a single gene. In certain embodiments, the up-regulation is of one or more genes listed in FIGS. 4 through 8. In certain embodiments, the cells in the biological sample are a single type of cell, such as blood cells (e.g., lymphocytes, such as B-lymphocytes, monocytes, or basophils). The cells may have been immortalized.

The present invention also provides a method for diagnosing a predisposition to, or likelihood of having, a substance use disorder or a mental illness or syndrome, where the method involves (a) determining a nucleic acid expression profile from a single type of cell from a biological sample from the subject; and (b) comparing the nucleic acid expression profile with a nucleic acid expression profile characteristic of the condition to determine if the patient has the a predisposition to, or likelihood of having, a substance use disorder or a mental illness or syndrome.

The present invention further provides a method for evaluating and treating a patient experiencing a substance use disorder or a mental illness or syndrome, where the method involves (a) obtaining a baseline laboratory profile comprising collecting blood from the patient to determine the patient's baseline nucleic acid expression profile level from a single type of cell; (b) treating the patient for the substance use disorder or a mental illness or syndrome; (c) obtaining a post-treatment laboratory profile comprising collecting blood from the patient to determine the patient's post-treatment nucleic acid expression profile level from the same type of cell tested previously; and (d) comparing the baseline and post-treatment laboratory profile to evaluate the effectiveness of the treatment.

The present invention provides a diagnostic method for determining a predisposition to, or likelihood of having, a substance use disorder or a mental illness or syndrome, where the method that involves comparing a test level value of at least three nucleic acid expression markers contained in a physiological sample from a subject suspected of having a predisposition to, or having, a substance use disorder or a mental illness or syndrome with a control level value of the at least three markers, wherein a test level value with a variance of more than the control level value of a combination of RNA levels or a test level value of more than the control level value of a combination of RNA levels is predictive of a predisposition to, or likelihood of having, a substance use disorder or a mental illness or syndrome in the subject.

### BRIEF DESCRIPTION OF THE FIGURES

FIGS. 1A and 1B. Validation of the AUTS2 and CAPN2 expression profiling using RTPCR. In order to validate the results of the microarray analyses, one marker from each of the 30 most highly up-regulated and down-regulated markers from Table 1A and 1B were evaluated using RT-PCR. AUTS2 expression (FIG. 1A) was significantly decreased (higher Z-score; ANOVA p<0.008) and the amount of CAPN2 expression (FIG. 1B) was significantly increased (lower Z-score; ANOVA p<0.004) in the six nicotine cases as compared to the nine controls.

FIG. 2. RTPCR analysis of KIAA1183, GYS2, DERL3 and TCP1 expression in PD cases (n=16) and well controls (n=15). The expression of TCP1 (p<0.001) (FIG. 2D), KIAA1183 (p<0.008) (FIG. 2A), and DERL3 (p<0.05) (FIG. 2C), but not GYS2 (p<0.30) (FIG. 2B) is significantly higher in the PD subjects than in the psychiatrically well controls (ANOVA p<0.001; i.e., lower Z score of the cycle count means higher amounts of expression).

FIG. 3. RTPCR analysis of COMT expression in PD males (n=6) male controls (n=6). Despite the microarray data, after controlling for LDHA and GAPDH levels, COMT levels were not significantly associated with affected status.

FIG. 4. (A) Down-regulated genes associated with nicotine dependence. (B) Up-regulated genes associated with nicotine dependence.

FIG. 5. (A) Down-regulated genes associated with panic disorder. (B) Up-regulated genes associated with panic disorder.

FIG. 6. (A) Down-regulated genes associated with nicotine dependence. (B) Up-regulated genes associated with Antisocial Personality Disorder (ASPD).

FIG. 7. (A) Down-regulated genes associated with panic disorder. (B) Up-regulated genes associated with Major Depressive Disorder (MDD).

FIG. 8. (A) Down-regulated genes associated with nicotine dependence. (B) Up-regulated genes associated with positive symptom schizophrenia such as that associated with the HOPA^{12bp} allele.

### DETAILED DESCRIPTION

The present invention provides methods to determine the nucleic acid expression profile of a patient in order to predict the clinical course and eventual outcome of patients suspected of being predisposed or of having a substance use disorder or a mental illness or syndrome. Previously, the only way to determine possible diagnoses was through subjective psychiatric evaluations. The present methods provide an objective component to diagnosis process.

Antisocial Personality Disorder

Antisocial personality disorder (ASPD) is a condition in which people show a pervasive disregard for the law and the rights of others. People with antisocial personality disorder may tend to lie or steal and often fail to fulfill job or parenting responsibilities. ASPD is a psychiatric diagnosis recognizable by the disordered individual's disregard for social rules and norms, impulsive behavior, and indifference to the rights and feelings of others. The terms “sociopath” and “psychopath” are sometimes used to describe a person with antisocial personality disorder.

Panic Disorder

Panic disorder (PD) is characterized by sudden attacks of terror, usually accompanied by a pounding heart, sweatiness, weakness, faintness, or dizziness. During these attacks, people with panic disorder may flush or feel chilled; their hands may tingle or feel numb; and they may experience nausea, chest pain, or smothering sensations. Panic attacks usually produce a sense of unreality, a fear of impending doom, or a fear of losing control. Panic disorder is often accompanied by other serious problems, such as depression, drug abuse, or alcoholism. These conditions need to be treated separately. Symptoms of depression include feelings of sadness or hopelessness, changes in appetite or sleep patterns, low energy, and difficulty concentrating. Most people with depression can be effectively treated with antidepressant medications, certain types of psychotherapy, or a combination of the two.

Major Depressive Disorder

Major Depressive Disorder (MDD) is a serious medical illness affecting 15 million American adults, or approximately 5 to 8 percent of the adult population in a given year. Unlike normal emotional experiences of sadness, loss, or passing mood states, major depression is persistent and can significantly interfere with an individual's thoughts, behavior, mood, activity, and physical health. Among all medical illnesses, major depression is the leading cause of disability in the U.S. and many other developed countries.

Depression occurs twice as frequently in women as in men, for reasons that are not fully understood. More than half of those who experience a single episode of depression will continue to have episodes that occur as frequently as once or even twice a year. Without treatment, the frequency of depressive illness as well as the severity of symptoms tends to increase over time. Left untreated, depression can lead to suicide.

Major depression, also known as clinical depression or unipolar depression, is only one type of depressive disorder. Other depressive disorders include dysthymia (chronic, less severe depression) and bipolar depression (the depressed phase of bipolar disorder or manic depression). People who have bipolar disorder experience both depression and mania. Mania involves unusually and persistently elevated mood or irritability, elevated self-esteem, and excessive energy, thoughts, and talking.

Nicotine Dependence

Nicotine dependence is the physical vulnerability of a person's body to the chemical nicotine, which is potently addicting when delivered by various tobacco products. Smoke from cigarettes, cigars and pipes contains thousands of chemicals, including nicotine. Nicotine is also found in chewing tobacco.

Positive Symptom Schizophrenia Such as that Associated with the HOPA^{12bp} Allele

Positive symptom schizophrenia such as that associated with the HOPA^{12bp} allele is a form of psychosis characterized by the presence of positive symptoms such as delusions or hallucinations in the presence of a relatively unimpaired concentration and a relative absence of negative symptoms. The terms “HOPA polypeptide” and “HOPA” when used herein encompass native sequence HOPA and HOPA polymorphic variants (see U.S. Pat. No. 6,566,061). HOPA polypeptides may be isolated from a variety of sources, such as from human tissue types or from another source, or prepared by recombinant or synthetic methods.

The term “psychotic disorder” when used herein is broadly defined as a mental disorder in which an individual loses contact with reality. Examples of psychotic disorders include, but are not limited to, schizophrenia, schizophreniform disorder, delusional disorder, schizoaffective disorder, and brief psychotic disorder. A psychotic disorder can be characterized by delusions, prominent hallucinations, disorganized speech, disorganized or catatonic behavior, etc.

The term “schizophrenia” when used herein is broadly defined as a mental disorder that is associated with psychosis and a decline in general functioning. This disorder is typically characterized by loss of contact with reality, hallucinations, delusions, abnormal thinking, disorganized speech, disorganized or catatonic behavior, and disrupted work and social functioning. The term “schizophrenia” includes all subtypes of schizophrenia including paranoid schizophrenia, disorganized schizophrenia, catatonic schizophrenia, undifferentiated schizophrenia, and residual schizophrenia.

The term “depression” when used herein is broadly defined as a depressed mood or loss of interest or pleasure in activities. The mood may be irritable rather than sad. Individuals suffering from depression typically experience additional symptoms including changes in appetite or weight, sleep and psychomotor activity; decreased energy; feelings of worthlessness or guilt; difficulty thinking, concentrating or making decisions; or recurrent thoughts of death or suicidal thoughts of death or suicidal ideation, plans or attempts.

In particular, in certain embodiments of the invention, the methods may be practiced as follows. A sample, such as a blood sample, is taken from a patient. In certain embodiments, a single cell type, e.g., lymphocytes, basophils, or monocytes isolated from the blood, may be isolated for further testing. The RNA is harvested from the sample and screened to determine whether certain mRNAs are up-regulated, and/or whether certain RNAs are down-regulated as compared to a control RNA value. A specific profile associates with a specific condition. As used herein the term “up-regulate” means that the quantity of the RNA from the sample is increases as compared to the amount of RNA from a control. This increase may be about 1% to 1000%, or any amount in-between, such as, for example, 1% 10%, 20%, 50%, 100%, 200%, 300%, 400%, 500%, 600, 700%, 800%, 900%, etc. As used herein the term “down-regulate” means that the quantity of RNA from the sample is decreased as compared to the level from a control. This down-regulation may be between 1-99.9% (i.e., the transcript may be totally eliminated).

Methods of determining the patient nucleic acid profile are well known to the art worker and include any of the well-known detection methods. Various PCR methods are described, for example, in PCR Primer: A Laboratory Manual, Dieffenbach 7 Dveksler, Eds., Cold Spring Harbor Laboratory Press, 1995. Other analysis methods include, but are not limited to, nucleic acid quantification, restriction enzyme digestion, DNA sequencing, hybridization technologies, such as Southern Blotting, etc., amplification methods such as Ligase Chain Reaction (LCR), Nucleic Acid Sequence Based Amplification (NASBA), Self-sustained Sequence Replication (SSR or 3SR), Strand Displacement Amplification (SDA), and Transcription Mediated Amplification (TMA), Quantitative PCR (qPCR), or other DNA analyses, as well as RT-PCR, in vitro translation, Northern blotting, and other RNA analyses. In another embodiment, hybridization on a microarray is used.

As used herein, the term “RNA probe” means a nucleic acid sequence that has at least about 80%, e.g., at least about 90%, e.g., at least about 95% contiguous sequence identity or homology to the RNA sequence encoding the targeted sequence of interest. A probe (or oligonucleotide or primer) of the invention has at least about 7-50, e.g., at least about 10-40, e.g., at least about 15-35, nucleotides. The oligonucleotide primers of the invention may comprise at least about seven nucleotides at the 3′ of the oligonucleotide primer that have at least about 80%, e.g., at least about 85%, e.g., at least about 90% contiguous identity to the targeted sequence of interest.

“Northern analysis” or “Northern blotting” is a method used to identify RNA sequences that hybridize to a known probe such as an oligonucleotide, DNA fragment, cDNA or fragment thereof, or RNA fragment. The probe is labeled with a radioisotope such as ³²P, by biotinylation or with an enzyme. The RNA to be analyzed can be usually electrophoretically separated on an agarose or polyacrylamide gel, transferred to nitrocellulose, nylon, or other suitable membrane, and hybridized with the probe, using standard techniques well known in the art.

“Stringent conditions” are those that (1) employ low ionic strength and high temperature for washing, for example, 0.015 M NaCl/0.0015 M sodium citrate (SSC); 0.1% sodium lauryl sulfate (SDS) at 50° C., or (2) employ a denaturing agent such as formamide during hybridization, e.g., 50% formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C. Another example is use of 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 0.1% SDS. Other examples of stringent conditions are well known in the art.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. The terms “nucleic acid,” “nucleic acid molecule,” or “polynucleotide” are used interchangeably and may also be used interchangeably with gene, cDNA, DNA and/or RNA encoded by a gene.

The term “nucleotide sequence” refers to a polymer of DNA or RNA which can be single-stranded or double-stranded, optionally containing synthetic, non-natural or altered nucleotide bases capable of incorporation into DNA or RNA polymers. A DNA molecule or polynucleotide is a polymer of deoxyribonucleotides (A, G, C, and T), and an RNA molecule or polynucleotide is a polymer of ribonucleotides (A, G, C and U).

A “gene,” for the purposes of the present disclosure, includes a DNA region encoding a gene product, as well as all DNA regions which regulate the production of the gene product, whether or not such regulatory sequences are adjacent to coding and/or transcribed sequences. The term “gene” is used broadly to refer to any segment of nucleic acid associated with a biological function. Genes include coding sequences and/or the regulatory sequences required for their expression. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. For example, “gene” refers to a nucleic acid fragment that expresses MRNA, functional RNA, or specific protein, including regulatory sequences. “Functional RNA” refers to sense RNA, antisense RNA, ribozyme RNA, siRNA, or other RNA that may not be translated but yet has an effect on at least one cellular process. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

“Gene expression” refers to the conversion of the information, contained in a gene, into a gene product. It refers to the transcription and/or translation of an endogenous gene, heterologous gene or nucleic acid segment, or a transgene in cells. In addition, expression refers to the transcription and stable accumulation of sense (mRNA) or functional RNA. Expression may also refer to the production of protein. The term “altered level of expression” refers to the level of expression in transgenic cells or organisms that differs from that of normal or untransformed cells or organisms.

A gene product can be the direct transcriptional product of a gene (e.g., MRNA, tRNA, rRNA, antisense RNA, ribozyme, structural RNA or any other type of RNA) or a protein produced by translation of an MRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation. The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (MRNA) refers to the RNA that is without introns and that can be translated into protein by the cell. “cDNA” refers to a single- or a double-stranded DNA that is complementary to and derived from mRNA.

A “coding sequence,” or a sequence that “encodes” a selected polypeptide, is a nucleic acid molecule that is transcribed (in the case of DNA) and translated (in the case of MRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral (e.g., DNA viruses and retroviruses) or prokaryotic DNA, and especially synthetic DNA sequences. A transcription termination sequence may be located 3′ to the coding sequence.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “fragment” is intended a polypeptide consisting of only a part of the intact full-length polypeptide sequence and structure. The fragment can include a C-terminal deletion an N-terminal deletion, and/or an internal deletion of the native polypeptide. A fragment of a protein will generally include at least about 5-10 contiguous amino acid residues of the full-length molecule, preferably at least about 15-25 contiguous amino acid residues of the full-length molecule, and most preferably at least about 20-50 or more contiguous amino acid residues of the full-length molecule, or any integer between 5 amino acids and the full-length sequence.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

“Naturally occurring” is used to describe a composition that can be found in nature as distinct from being artificially produced. For example, a nucleotide sequence present in an organism, which can be isolated from a source in nature and which has not been intentionally modified by a person in the laboratory, is naturally occurring.

“Functional RNA” refers to sense RNA, antisense RNA, ribozyme RNA, siRNA, or other RNA that may not be translated but yet has an effect on at least one cellular process.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (MRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences.

A “5′ non-coding sequence” refers to a nucleotide sequence located 5′ (upstream) to the coding sequence. It is present in the fully processed MRNA upstream of the initiation codon and may affect processing of the primary transcript to MRNA, MRNA stability or translation efficiency.

A “3′ non-coding sequence” refers to nucleotide sequences located 3′ (downstream) to a coding sequence and may include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed MRNA upstream (5′) of the translation start codon. The translation leader sequence may affect processing of the primary transcript to mRNA, mRNA stability or translation efficiency.

A “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

“Constitutive expression” refers to expression using a constitutive promoter. “Conditional” and “regulated expression” refer to expression controlled by a regulated promoter.

“Operably-linked” refers to the association of nucleic acid sequences on a single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. In addition, expression refers to the transcription and stable accumulation of sense (MRNA) or functional RNA. Expression may also refer to the production of protein.

The term “altered level of expression” refers to the level of expression in cells or organisms that differs from that of normal cells or organisms.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well-known in the art. Thus, the determination of percent identity between any two sequences can be accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and. TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) can be utilized. Alternatively, PSI-BLAST (in BLAST 2.0) can be used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity to the promoter sequences disclosed herein may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, 80%, 90%, or even at least 95%.

Another indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even 95%, 96%, 97%, 98% or 99%, sequence identity to the reference sequence over a specified comparison window. In certain embodiments, optimal alignment is conducted using the homology alignment algorithm of Needleman and Wunsch (Needleman and Wunsch, JMB, 48, 443 (1970)). An indication that two peptide sequences are substantially identical is that one peptide is immunologically reactive with antibodies raised against the second peptide. Thus, a peptide is substantially identical to a second peptide, for example, where the two peptides differ only by a conservative substitution. Thus, the invention also provides nucleic acid molecules and peptides that are substantially identical to the nucleic acid molecules and peptides presented herein.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl (1984); Tₘ81.5° C.+16.6(log M)+0.41(% GC)−0.61(% form)−500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with>90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), the SSC concentration is increased so that a higher temperature can be used. Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes. Often, a high stringency wash is preceded by a low stringency wash to remove background probe signal. An example medium stringency wash for a duplex of, e.g., more than 100 nucleotides, is 1×SSC at 45° C. for 15 minutes. For short nucleotide sequences (e.g., about 10 to 50 nucleotides), stringent conditions typically involve salt concentrations of less than about 1.5 M, less than about 0.01 to 1.0 M, Na ion concentration (or other salts) at pH 7.0 to 8.3, and the temperature is typically at least about 30° C. and at least about 60° C. for long probes (e.g.,>50 nucleotides). Stringent conditions may also be achieved with the addition of destabilizing agents such as formamide. In general, a signal to noise ratio of 2×(or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids that have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1 M NaCl, 1% SDS (sodium dodecyl sulphate) at 37° C., and a wash in 133 to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

In a further embodiment of the invention, there are provided articles of manufacture and kits containing probes, oligonucleotides or antibodies which can be used, for instance, for the diagnostic applications described above. The article of manufacture comprises a container with a label. Suitable containers include, for example, bottles, vials, and test tubes. The containers may be formed from a variety of materials such as glass or plastic. The container holds a composition which includes an agent that is effective for diagnostic applications, such as described above. The label on the container indicates that the composition is used for a specific diagnostic application. The kit of the invention will typically comprise the container described above and one or more other containers comprising materials desirable from a commercial and user standpoint, including buffers, diluents, filters and package inserts with instructions for use.

The probes of the present invention can be labeled using techniques known to those of skill in the art. For example, the labels used in the assays of invention can be primary labels (where the label comprises an element that is detected directly) or secondary labels (where the detected label binds to a primary label, e.g., as is common in immunological labeling). An introduction to labels (also called “tags”), tagging or labeling procedures, and detection of labels is found in Polak and Van Noorden (1997) Introduction to Immunocytochemistry, second edition, Springer Verlag, N.Y. and in Haugland (1996) Handbook of Fluorescent Probes and Research Chemicals, a combined handbook and catalogue Published by Molecular Probes, Inc., Eugene, Oreg. Primary and secondary labels can include undetected elements as well as detected elements. Useful primary and secondary labels in the present invention can include spectral labels such as fluorescent dyes (e.g., fluorescein and derivatives such as fluorescein isothiocyanate (FITC) and Oregon Green™, rhodamine and derivatives (e.g., Texas red, tetramethylrhodamine isothiocyanate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P), enzymes (e.g., horse-radish peroxidase, alkaline phosphatase) spectral calorimetric labels such as colloidal gold or colored glass or plastic (e.g., polystyrene, polypropylene, latex) beads. The label may be coupled directly or indirectly to a component of the detection assay (e.g., the labeled nucleic acid) according to methods well known in the art. As indicated above, a wide variety of labels may be used, with the choice of label depending on sensitivity required, ease of conjugation with the compound, stability requirements, available instrumentation, and disposal provisions. In general, a detector that monitors a probe-substrate nucleic acid hybridization is adapted to the particular label that is used. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeled nucleic acids is digitized for subsequent computer analysis.

Preferred labels include those that use (1) chemiluminescence (using Horseradish Peroxidase and/or Alkaline Phosphatase with substrates that produce photons as breakdown products) with kits being available, e.g., from Molecular Probes, Amersham, Boehringer-Mannheim, and Life Technologies/Gibco BRL; (2) color production (using both Horseradish Peroxidase and/or Alkaline Phosphatase with substrates that produce a colored precipitate) (kits available from Life Technologies/Gibco BRL, and Boehringer-Mannheim); (3) hemifluorescence using, e.g., Alkaline Phosphatase and the substrate AttoPhos (Amersham) or other substrates that produce fluorescent products, (4) Fluorescence (e.g., using Cy-5 (Amersham), fluorescein, and other fluorescent labels); (5) radioactivity using kinase enzymes or other end-labeling approaches, nick translation, random priming, or PCR to incorporate radioactive molecules into the labeled nucleic acid. Other methods for labeling and detection will be readily apparent to one skilled in the art.

Fluorescent labels are highly preferred labels, having the advantage of requiring fewer precautions in handling, and being amendable to high-throughput visualization techniques (optical analysis including digitization of the image for analysis in an integrated system comprising a computer). Preferred labels are typically characterized by one or more of the following: high sensitivity, high stability, low background, low environmental sensitivity and high specificity in labeling. Fluorescent moieties, which are incorporated into the labels of the invention, are generally are known, including Texas red, dixogenin, biotin, 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bis-benzoxazole, bis-p-oxazolyl benzene, 1,2-benzophenazin, retinol, bis-3-aminopyridinium salts, hellebrigenin, tetracycline, sterophenol, benzimidazolylphenylamine, 2-oxo-3-chromen, indole, xanthen, 7-hydroxycoumarin, phenoxazine, calicylate, strophanthidin, porphyrins, triarylmethanes, flavin and many others. Many fluorescent labels are commercially available from the SIGMA Chemical Company (Saint Louis, Mo.), Molecular Probes, R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka ChemicaBiochemika Analytika (Fluka Chemie AG, Buchs, Switzerland), and Applied Biosystems (Foster City, Calif.), as well as many other commercial sources known to one of skill.

Means of detecting and quantifying labels are well known to those of skill in the art. Thus, for example, where the label is a radioactive label, means for detection include a scintillation counter or photographic film as in autoradiography. Where the label is optically detectable, typical detectors include microscopes, cameras, phototubes and photodiodes and many other detection systems that are widely available.

The present invention is further detailed in the following Examples, which are offered by way of illustration and are not intended to limit the invention in any manner. Standard techniques well known in the art or the techniques specifically described below are utilized. All patent and literature references cited in the present specification are hereby incorporated by reference in their entirety.

### EXAMPLE 1

### Lymphoblast Expression Profiling

The Iowa Adoption Studies (IAS) are a longitudinal case and control study founded by Dr. Remi Cadoret that use the adoption paradigm to examine the role of genetic (G), environmental (E) and gene-environment (G×E) interaction effects in the development and maintenance of depression and substance use in a group of 950 adoptees from the State of Iowa (Yates et al., The Iowa Adoption Studies Methods and Results. In: LaBuda M, Grigorenko E, editors; 1998. p. 95-125). Four prior waves of structured interviews, spaced approximately five years apart, have characterized the behavior and adoptive environment of each of the IAS subjects. The analysis of the data from these interviews has provided a great deal of insight in the separate, but vital role that G, E and G×E factors play in depression and the substance use disorders. In the current fifth wave, researchers are re-interviewing and phlebotomizing these subjects in an attempt to add a molecular component to these studies.

In addition, the inventors prepared lymphoblast cell lines in order to provide DNA for genotyping and MRNA for expression analysis. The DNA from these cell lines is commonly used for genetic studies by a large number of investigators. However, though these lymphoblast cell lines are commonly used as models for serotonin transporter regulation (Bradley et al., American Journal of Medical Genetics, 136B:58-61, 2005; Hranilovic et al., Biol Psychiatry 2004; 55(11):1090-1094), whether the mRNA derived from these cell lines is useful for other purposes was not known.

Morello and colleagues have shown that lymphoblast cell lines from subjects with hyperlipidemia retain the transcriptional profile of the subjects from whom they were derived (Arterioscler Thromb Vasc Biol 2004; 24(11):2149-2154). Furthermore, a recent review has highlighted the utility of lymphoblasts in deciphering the biology of complex non-behavioral disorders (Liew et al., J Lab Clin Med 147(3):126-32, 2006). The inventors investigated whether gene expression in lymphoblast cell lines could provide insight into the biology of substance use disorders and mental disorders.

To examine this question, the inventors first performed genome-wide expression profiling in lymphoblast cell lines derived from a subset of six nicotine dependent subject and nine matched controls without a history of substance use or depression to identify transcripts differentially expressed in cases and controls. The inventors then followed the expression of several of the more interesting candidate genes using real-time PCR (RT-PCR) extension in these 15 lines and in an additional 79 lines. The inventors analyzed the resulting data with respect to clinical information from wave four and five (1999-2003; 2004-current) of the IAS.

Transcriptional profiling has been used to identify gene expression patterns indicative of general medical illnesses such as atherosclerosis. However, whether these methods can identify more common psychiatric disorders and other complex disorders has not been established. To answer this question with respect to nicotine use, the inventors used genome wide expression profiling lymphoblast cell lines from six actively-smoking IAS subjects and nine “clean” control subjects, followed by RT-PCR of gene expression to examine gene expression patterns in lymphoblast-derived RNA from 94 subjects in the IAS. As compared to controls without a history of smoking (n=9), the expression levels 579 of 29,098 genes were significantly up-regulated, and expression levels of 584 of 29,098 genes were significantly down-regulated in lymphoblast lines from currently smoking subjects (n=6). RT-PCR confirmation of three select RNA levels confirmed the validity of the overall profile and revealed highly significant relationships between the expression of some of these transcripts and 1) major depression, 2) antisocial personality disorder, 3) nicotine dependence, and 3) cannabis dependence. The inventors concluded that the use of lymphoblast expression profiling contributes significant insights into the biology of complex behavioral disorders.

### METHODS

The overall design and methodology of the Iowa Adoption Studies and the complete demographic and clinical cohorts were gathered have been described in detail previously (Yates et al., (1998) The Iowa Adoption Studies Methods and Results. On the way to individuality: Methodological Issues in Behavioral Genetics. M. LaBuda and E. Grigorenko. Hauppauge N.Y., Nova Science Publishers: 95-125). All clinical data used in this study was derived from structured interviews using the Structured Assessment for the Genetic Studies of Alcoholism (SSAGA) (Bucholz et al., J Stud Alcohol 1994; 55(2):149-58). Symptom counts for major depression (maximum score of 9), antisocial personality disorder (maximum score of 7) and individual substance dependence (maximum score of 7) were derived from the SSAGA data using criteria from the DSM-IV (Association 1994. Diagnostic and Statistical Manual of Mental Disorders, Fourth Edition. Washington D.C., American Psychiatric Association) as per our previous methods (Yates et al., 1996, Drug and Alcohol Dependence 41(1): 9). Current smoking status was defined as smoking one or more cigarettes or cigars on a daily basis. Phlebotomy and interviewing was performed by a trained research assistant blind to clinical status. All procedures and methods were approved by the University of Iowa Institutional Review Board for Human Subjects.

The lymphoblast cell lines used for this study were derived using standard EBV transfection methods (Ginns et al., Nat Genet 1996; 12(4):431-5) and grown using standard bovine serum based growth media supplemented with L-glutamine and penicillin/streptomycin. Media was changed for each of the cell lines 24 hours prior to extraction.

As a first step, RNA from these lines was prepared using an Invitrogen™ RNA purification kit (Invitrogen™, Carlsbad, Calif.) according to the instructions provided by the manufacturer and stored in Ambion® RNA storage solution at −80° C. All samples were examined spectrophotometrically to ensure quality with a subset of the samples being further analyzed using an Agilent Bioanalyzer.

From the first 94 subjects with complete biological and clinical data, the inventors selected six subjects (four male, two female) with active nicotine dependence and nine subjects (six male, three female) without a history of substance use or depression. The inventors then performed microarray analysis on RNA extracted from their lymphoblast cell lines. To do this, total RNA was extracted using an Invitrogen™ total RNA purification kit. Quality and integrity of the RNA samples was verified by Agilent Bioanalyzer. Total RNA (2 μg) was reverse-transcribed with the Chemiluminescent RT-IVT Labeling Kit (Applied Biosystems) and was hybridized to the ABI 1700 Human Genome Expression Microarray containing 29,098 gene-specific 60-mer oligonucleotide probes per manufacturer's protocol. Data was quantile normalized and t-test was applied to each gene for statistical significance and to provide P-values. Unsupervised hierarchical clustering was performed using Spotfire® software to classify genes that have similar expression patterns. The Euclidean distance was used as a similarity method, based on gene expression levels, with no gene pre-selected. WPGMA (weighted average) was used to cluster genes. Storey q-value method was then use to assess false discovery rate to determine the number of genes differentially expressed in 80% of the cell lines and to provide q-values (Storey et al., Methods Mol Biol 2003; 224:149-57). The significantly up-regulated (n=579) and down-regulated (n=584) gene lists represent those genes whose expression was arithmetically changed in the same direction (i.e., up or down) in each of the cases and significantly changed as compared to the controls as described above. All differentially expressed genes were selected at the false discovery rate of 5% per the method of Storey (Storey et al., Methods Mol Biol 2003; 224:149-57).

From these differentially affected mRNAs, the inventors chose two mRNAs [5HTT (or SLC6A4) and ELN] for further examination based on their biological plausibility and three mRNAs from the list of 30 most up-regulated or down-regulated transcripts (AUTS2, RPS19 and CAPN2; see Table 1A and 1B) for further RT-PCR examination in all 94 lymphoblast cell lines available. As a first step in this process, the inventors converted 5 μg aliquots of total RNA samples from each cell line to cDNA using an Applied Biosystems cDNA Archiving Kit (Applied Biosystems, Foster City, Calif.) according to manufacturer's directions. The resulting cDNA solutions were then diluted and aliquoted into master plates for robotic liquid handling.

Quantitative RTPCR quantification of gene expression for these five transcripts was then performed using Taqman® Universal Master Mix Reagents (PE Biosystems, Branchburg, N.J.) in combination with Assays-on-Demand® primer/probe sets specific for the ELN, AUTS2, RPS 19, CAPN2 and 5HTT transcripts and an ABI 7900HT Sequence Detection System. Relative levels of these RNAs were determined by the comparative C_{T} method using LDHA and GAPDH as normalizing controls (Dhed et al., Biotechniques 2004; 37(1):112-4, 116, 118-9). Z-scores for each RT-PCR run were calculated and all runs were performed in duplicate. Although RTPCR using the RPS19 primer probe set gave excellent signal, the correlation coefficient between duplicate samples was very low (<85%; normally this is>99%). Therefore, the results using this marker were excluded from further analyses. The resulting genetic (continuous variables of RNA expression Z-scores) and clinical data (e.g., ordinal symptom counts) were analyzed using JMP (version 5.1; SAS Institute, Cary, N.C., USA) using ordinal logistic regression using single markers or multiple RNA expression levels as indicated in the text. When testing whether more than one marker would better fit the model, the significance of the Effect Wald test from the all four markers together test (5HTT, ELN, AUTS2 and CAPN2-Table IV) was used to determine whether a marker was retained in the “Best Markers Only” model.

Two types of pathway analyses were conducted. The first was an unsupervised clustering analysis of the differentially expressed genes to known pathways using the Panther analysis subroutine and default settings (Applied Biosystems, see world-wide-web at pantherdb.org) and the binomial test as described by Cho and Campbell (Trends Genet 16(9): 409-15). The second analysis was conducted using Gene Ontology subroutine (world-wide-web at godatabase.org/dev) (Feng et al., 2003 AMIA Annu Symp, Proc: 839). Briefly, using the recently developed GoMiner program, a GO category structure based on genes currently carrying human GO annotations was constructed and used as Query Gene File (Zeeberg et al. 2003 Genome Biol 4(4): R28). The resulting file was loaded into the GoMiner program to examine the distribution of these genes in the GO category structure.

The microarray data has been submitted to the NCBI GEO database. The accession numbers are GSM144125 through GSM144136.

### RESULTS

As a first step, the inventors performed microarray analysis on RNA extracted from lymphoblast cell lines derived from six subjects with active nicotine dependence and nine controls without a history of substance abuse or major depression selected from the first 94 lymphoblast cell lines derived from the Iowa Adoption Studies. Four of the currently smoking nicotine cases were completely medication free, a fifth was on risperidone, while the sixth nicotine case was on atorvastatin and used albuterol, monteleukast, and fluticasone inhalers. Four of the controls were also medication free, one was atorvastatin, atenolol and lisinopril; one was on atenolol; one was on naproxen, clopidogrel, and metoprolol; and one was on azathioprine, atorvastatin, glyburide-metformin, rosiglitazone, amlodipine and enalopril. Overall, only one subject, the nicotine case who was prescribed risperidone, was on medications for psychiatric reasons.

The inventors performed an unsupervised hierarchical clustering of normalized expression levels of genes in lymphoblast cell lines. The expression levels of 579 genes were significantly up-regulated while the expression levels of 584 genes were significantly down-regulated in the lymphoblast cell lines from the six nicotine dependent subjects as compared to those from the nine “clean” controls. Tables 1A and 1B give a list of the top 30 most up-regulated and the 30 most down-regulated transcripts.

TABLE 1A
The Thirty Most Down-Regulated Genes.

| Probe ID | Gene Symbol | Cytoband | Panther_Process | GO-Cellular-Component | fold_change | p-value | q-value |
|---|---|---|---|---|---|---|---|
| 212042 | RPS19 | 19q13.2 | Protein metabolism and modification | | −3.69 | p<0.010 | q<0.047 |
| 128153 | COX5B | 2cen-q13 | Electron transport | mitochondrial membrane | −3.63 | p<0.007 | q<0.040 |
| 174637 | | | Protein metabolism and modification | | −3.48 | p<0.010 | q<0.049 |
| 214080 | | | | | −3.41 | p<0.003 | q<0.028 |
| 118887 | | | Protein metabolism and modification | | −3.33 | p<0.003 | q<0.028 |
| 114043 | | | Muscle contraction | | −3.28 | p<0.006 | q<0.039 |
| 153002 | RPS10 | 6p21.31 | small ribosomal subunit | | −3.26 | p<0.008 | q<0.045 |
| 228451 | | | | | −3.21 | p<0.010 | q<0.048 |
| 206935 | MRPL23 | 11p15.5-p15.4 | Protein metabolism & modification | ribosome | −3.15 | p<0.001 | q<0.023 |
| 130943 | RPL10A | 6p21.3-p2l.2 | Protein metabolism & modification | | −3.07 | p<0.005 | q<0.037 |
| 226948 | HIST1H4I | 6p21.33 | Nucleoside, nucleotide & nucleic | | −3.05 | p<0.010 | q<0.048 |
| | | | acid metabolism | | | | |
| 171120 | | | | | −3.03 | p<0.005 | q<0.035 |
| 119116 | COMMD6 | | | | −2.95 | p<0.003 | q<0.027 |
| 201308 | | | Protein metabolism & modification | | −2.93 | p<0.009 | q<0.047 |
| 146877 | | | Lipid, fatty acid & steroid metabolism | | −2.85 | p<0.004 | q<0.032 |
| 106007 | | | | | −2.85 | p<0.008 | q<0.045 |
| 225790 | GAMT | 19p13.3 | Biological process unclassified | | −2.84 | p<0.008 | q<0.043 |
| 127866 | | | | | −2.75 | p<0.010 | q<0.048 |
| 130996 | | | | | −2.72 | p<0.005 | q<0.035 |
| 158641 | CKLFSF7 | 3p22.3 | Biological process unclassified | integral to membrane | −2.71 | p<0.002 | q<0.025 |
| 234453 | | | Protein metabolism & modification | | −2.69 | p<0.007 | q<0.040 |
| 170400 | | | | | −2.69 | p<0.006 | q<0.039 |
| 176535 | | | | | −2.65 | p<0.009 | q<0.045 |
| 154419 | DULLARD | 17p13.2; | Developmental processes | plasma membrane | −2.64 | p<0.001 | q<0.019 |
| 128466 | | | | | −2.59 | p<0.002 | q<0.027 |
| 165281 | | | | | −2.59 | p<0.007 | q<0.043 |
| 187132 | TRAPPC1 | 17p13.2 | Biological process unclassified | endoplasmic reticulum | −2.57 | p<0.003 | q<0.027 |
| 208933 | | | Biological process unclassified | | −2.56 | p<0.003 | q<0.028 |
| 214094 | RPL23A | 17q11 | Protein metabolism & modification | ribosome | −2.50 | p<0.010 | q<0.048 |
| 209685 | | | Biological process unclassified | | −2.48 | p<0.002 | q<0.026 |

Gene name is per GenBank.
The extent of up-regulation is expressed as the “fold” of the net change. Where known, the function of the gene according to Panther analytic or Gene Ontogeny subroutine is given as described in the methods.
P-values and q-values were calculated as described in the methods section.

TABLE 1B
The Thirty Most Up-Regulated Genes.

| Probe ID | Gene Symbol | Cytoband | Panther Process | GO-Cellular-Component | fold-change | p-value | q-value |
|---|---|---|---|---|---|---|---|
| 103642 | | | | | 5.76 | 0.002 | 0.025 |
| 202763 | | | | | 4.63 | 0.005 | 0.035 |
| 182098 | | | | Sigl transduction | 4.08 | 0.006 | 0.040 |
| 210742 | CAPN2 | 1q41-q42 | Protein metabolism and modification intracellular | | 3.65 | 0.000 | 0.011 |
| 135433 | MARVELD3 | 16q22.2 | | membrane | 3.64 | 0.004 | 0.031 |
| 149398 | CAPN2 | 1q41-q42 | Protein metabolism and modification | intracellular | 3.42 | 0.000 | 0.013 |
| 236527 | | | | | 3.34 | 0.007 | 0.040 |
| 220463 | | | | | 3.33 | 0.009 | 0.046 |
| 103654 | | | | | 3.31 | 0.007 | 0.040 |
| 182347 | | | | | 3.09 | 0.006 | 0.038 |
| 175689 | | | Cell structure and motility | | 3.08 | 0.002 | 0.027 |
| 223790 | PCDHGB7 | 5q31 | Sigl transduction- | integral to membrane | 3.06 | 0.002 | 0.026 |
| 225245 | | | | | 3.03 | 0.009 | 0.047 |
| 152537 | LCN9 | 9q34.3 | Transport- | | 2.95 | 0.001 | 0.023 |
| 113849 | | | | | 2.81 | 0.011 | 0.049 |
| 233070 | | | | | 2.63 | 0.003 | 0.030 |
| 191441 | RSAD2 | 2p25.2 | Coenzyme and prosthetic group metabolism | | 2.62 | 0.000 | 0.008 |
| 118637 | DCN | 12q13.2 | Developmental processes | extracellular matrix | 2.61 | 0.004 | 0.032 |
| 105004 | KCTD12 | 13q22.1 | Biological process unclassified: | membrane | 2.60 | 0.008 | 0.045 |
| 179831 | FLJ90586 | 7q35 | | | 2.58 | 0.006 | 0.039 |
| 103193 | | | Biological process unclassified | | 2.54 | 0.011 | 0.050 |
| 234834 | | | | | 2.50 | 0.003 | 0.028 |
| 164423 | | | | | 2.49 | 0.003 | 0.030 |
| 101575 | | | | | 2.43 | 0.005 | 0.035 |
| 189694 | COCH | 14q12-q13 | Sensory perception | | 2.39 | 0.004 | 0.032 |
| 118644 | MLKL | 16q22.3 | Biological process unclassified | | 2.35 | 0.001 | 0.018 |
| 115055 | C2orf26 | 2q13 | Biological process unclassified | | 2.34 | 0.008 | 0.043 |
| 164834 | | | Immunity and defense | | 2.34 | 0.003 | 0.028 |
| 198965 | LILRB4 | 19q13.4 | Immunity and defense | integral to membrane | 2.34 | 0.000 | 0.011 |
| 172527 | GRK4 | 4p16.3 | Protein metabolism and modification | | 2.34 | 0.008 | 0.045 |

Gene name is per GenBank.
The extent of up-regulation is expressed as the “fold” of the net change. Where known, the function of the gene according to Panther analytic or Gene Ontogeny subroutine is given as described in the methods.
P-values and q-values were calculated as described in the methods section.

The inventors compared genes that were differentially expressed in those with active nicotine dependence with the genes present in 107 regulatory and metabolic pathways using the Panther pathway analysis program (see world-wide-web at pantherdb.org). These pathways represent about 12.0% of the known genes present in public databases. Of the 1163 genes differentially regulated those subjects with active nicotine dependence, only 97 of them mapped into identified Panther pathways. In particular, a large number of these genes mapped into apoptosis signaling (n=9), FAS signaling (n=3), p53 pathway (n=9), TGF-beta (n=7), de novo purine biosynthesis (n=5), G-protein signaling (n=8) and Alzheimer disease-presenilin (n=7) pathways.

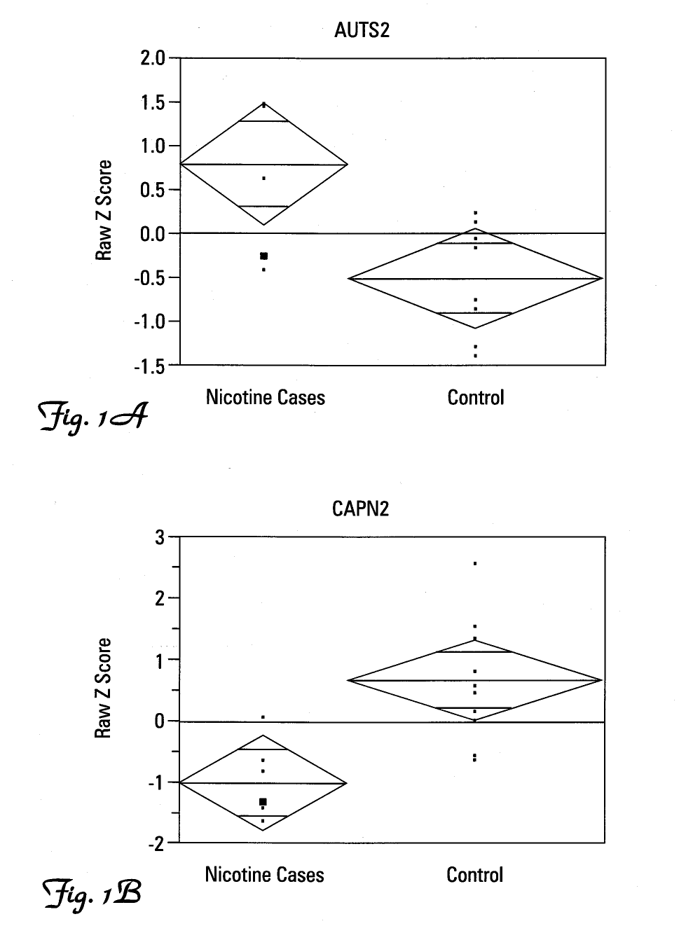

As a second step in the analysis, the inventors tested the validity of the genome-wide transcription profiling using RTPCR and one marker from the list top 30 most up-regulated and the 30 most down-regulated transcripts. As FIG. 1 demonstrates, the amount of AUTS2 expression was significantly decreased (higher Z-score; ANOVA p<0.008) and the amount of CAPN2 was significantly increased (lower Z-score; ANOVA p<0.004) in the six nicotine cases as compared to the nine controls.

From the total list of up-regulated or down-regulated genes, the inventors chose two transcripts for further examination based on a number of factors including biological plausibility, our prior interest, physical clustering of differential expressed genes, and the degree of differential expression. 5HTT (SLAC6A4) the serotonin reuptake transporter was chosen because of its obvious plausibility. Elastin (ELN) was chosen because its localization to 7q11, a linkage peak for panic disorder (Crowe et al., 2001 American Journal of Medical Genetics 105(1):105-9), a disorder whose frequency is increased in subjects with nicotine dependence (Insensee et al. 2003 Arch Gen Psychiatry 60(7): 692-700).

In order to extend and replicate this profile, we prepared RNA from the 79 additional lymphoblast lines derived from subjects from the Iowa Adoption Studies. Table 2 gives the distribution of the DSM IV symptom counts for all 94 of the corresponding subjects for each of the most common behavioral disorders, major depressive disorder (MDD), antisocial personality disorder (ASPD), nicotine dependence (Nicotine Dep.), alcohol dependence (Alcohol Dep.) and Cannabis dependence (Cannabis Dep.) found in the Iowa Adoption Studies. Consistent with the design of this adoption study in which half of the participants are intentionally enrolled because of the strong history of MDD, ASPD and substance use in their biological parents (Yates et al., 1998 Drug and Alcohol Dependence 41(1): 9), high symptom counts for MDD, ASPD and substance use are relatively frequent in these 94 adoptee subjects.

TABLE 2
DSM IV SYMPTOM COUNT FOR THE COMMON DISORDERS

| | N | # of Sx | MDD | ASPD | Nicotine Dep. | Alcohol Dep. | Cannabis Dep. |
|---|---|---|---|---|---|---|---|
| Men | 38 | | | | | | |
| | | 0 | 0 | 6 | 24 | 15 | 30 |
| | | 1 | 27 | 10 | 4 | 9 | 2 |
| | | 2 | 2 | 9 | 1 | 8 | 0 |
| | | 3 | 2 | 6 | 3 | 0 | 3 |
| | | 4 | 0 | 3 | 3 | 2 | 1 |
| | | 5 | 1 | 0 | 2 | 3 | 1 |
| | | 6 | 3 | 4 | 1 | 1 | 1 |
| | | 7 | 1 | 0 | 0 | 0 | 0 |
| | | 8 | 2 | | | | |
| | | 9 | 0 | | | | |
| Women | 56 | | | | | | |
| | | 0 | 0 | 25 | 29 | 37 | 51 |
| | | 1 | 25 | 14 | 3 | 9 | 0 |
| | | 2 | 0 | 8 | 4 | 7 | 1 |
| | | 3 | 2 | 5 | 5 | 1 | 0 |
| | | 4 | 2 | 2 | 11 | 0 | 0 |
| | | 5 | 2 | 0 | 2 | 1 | 2 |
| | | 6 | 7 | 1 | 2 | 0 | 2 |
| | | 7 | 9 | 1 | 0 | 1 | 0 |
| | | 8 | 4 | | | | |
| | | 9 | 5 | | | | |

Symptom counts for major depression (MDD, maximum score of 9), antisocial personality disorder (ASPD, maximum score of 7) and individual substance dependence (maximum score of 7) were derived from the SSAGA data using criteria from the DSM-IV (Association 1994, Diagnostic and Statistical Manual of Mental Disorder, Fourth Edition. Washington D.C., American Psychiatric Association) as per previous methods (Yates et al. 1996, Drug and Alcohol Dependence 41(1): 9).

Since prior studies have shown that these common behavioral disorders have partially overlapping genetic and environmental diatheses, the inventors analyzed the relationship of these variables using Spearman's Rho for ordinal variables. As Table 3 illustrates, each of the substance dependence count variables were significantly correlated with one another and ASPD symptom counts were significantly correlated with both alcohol and cannabis dependence symptom counts. Surprisingly, lifetime symptom counts for MDD were not significantly related to any of the other symptoms.

TABLE 3
Spearman's Rho Correlation of Symptom Counts

| Variable | by Variable | Spearman Rho | |
|---|---|---|---|
| Prob>\|Rho\| | | | |
| Nicotine Dep. Symptom | MDD Symptom | 0.0897 | 0.3902 |
| Alcohol Dep. Symptom | MDD Symptom | 0.1285 | 0.2172 |
| Alcohol Dep. Symptom | Nicotine Dep. Symptom | 0.3521 | 0.0005 |
| Cannabis Dep. Symptom | MDD Symptom | 0.0236 | 0.8212 |
| Cannabis Dep. Symptom | Nicotine Dep. Symptom | 0.2596 | 0.0115 |
| Cannabis Dep. Symptom | Alcohol Dep. Symptom | 0.5453 | <.0001 |
| ASPD Symptom Ct | MDD Symptom | 0.0862 | 0.4089 |
| ASPD Symptom Ct | Nicotine Dep. Symptom | 0.1998 | 0.0535 |
| ASPD Symptom Ct | Alcohol Dep. Symptom | 0.6087 | <.0001 |
| ASPD Symptom Ct | Cannabis Dep. Symptom | 0.5059 | <.0001 |

The inventors then analyzed the relationship between these symptom counts and individual RNA expression levels using RT-PCR (Table 4). Contrary to initial expectations given the results of the microarray analysis, the relationship between both AUTS2 and CAPN2 expression levels and nicotine dependence was not significant, but the relationships between AUTS2 and CAPN2 expression levels and cannabis dependence were highly significant. Retrospective analysis of the six nicotine “case” samples shed light on this matter. Four of the six nicotine cases had significant histories of cannabis dependence (4 of the 13 (31%) with cannabis dependence symptoms in the sample). In contrast, these six samples only represented 15% of those 41 individuals in the sample who had some symptoms of nicotine dependence, which suggest that in some respects these six nicotine “cases” were actually better cases for cannabis dependence. AUTS2 expression was also highly correlated with ASPD symptom count as well. ELN expression was significantly correlated with MDD and nicotine symptom counts, as well as the current smoker status. By itself, 5HTT expression was not correlated with any syndrome.

The inventors next tested to see if using information from all the markers or markers with evidence of association, as evidenced by significant individual Effect Wald tests on the whole model test, would increase the significance of the relationship of gene expression and symptom counts using multiple ordinal regression (Table 4). Highly significant relationships could be found between two marker panel sets for current smoking status and both nicotine and cannabis symptom counts. But the addition of other markers did not improve the significance of the relationships between ELN and CAPN2 expression and MDD and ASPD symptom counts, respectively.

TABLE 4
Ordinal Regression Analysis of RNA expression levels and Symptom Counts.

| Condition | Individual Marker Tests | | | | All Markers | Best Markers |
|---|---|---|---|---|---|---|
| Marker Name | 5HTT | AUTS2 | ELN | CAPN2 | Together | Only |
| MDD Symptom Count | p<0.96 | p<0.58 | p<0.02 | p<0.91 | p<0.13 | p<0.02 (ELN only) |
| Nicotine Dep. Symptom Count | p<0.21 | p<0.44 | p<0.003 | p<0.39 | p<0.003 | p<0.0005 (ELN, 5HTT) |
| Alcohol Dep. Symptom Count | p<0.32 | p<0.06 | p<0.89 | p<0.19 | p<0.16 | p<0.04 (ELN, AUTS2) |
| Cannabis Dep. Symptom Count | p<0.51 | p<0.002 | p<0.57 | p<0.002 | p<0.0004 | p<0.0001 (AUTS2, CAPN2) |
| ASPD Symptom Count | p<0.25 | p<0.0001 | p<0.97 | p<0.97 | p<0.0003 | p<0.0001 (CAPN2) |
| Current Smoker | p<0.36 | p<0.10 | p<0.001 | p<0.93 | p<0.0006 | p<0.0003 (ELN, AUTS2) |

*Symptom counts and Current Smoker status are derived from the SSAGA using DSM-IV criteria as described in the methods section.
All p values are uncorrected for multiple comparisons. A detailed description of the analytic techniques is given in the methods section.

### DISCUSSION

In summary, the inventors report that the expression of select genes in RNA obtained from lymphoblast cell lines is correlated with lifetime symptom counts of major depression, alcohol dependence, cannabis dependence and nicotine dependence.

Before discussing the data, several caveats should be noted. First, since data for each of these subjects from the most recent interview are not fully available, all clinical information with the exception of current nicotine use was derived from structured interviews performed two to five years before the lymphoblast immortalization. However, our hand check of these interviews demonstrates that the information is highly accurate and that no significant changes in substance use or vulnerability to depression have occurred. Second, the clinical information is not independently verified (i.e., urine drug tests). Therefore, it is quite possible that some of clinical information may be inaccurate. Third, the inventors have not fully accounted for the possible effects of medications on gene expression. But it is noted that a) most of the subjects are not on psychiatric medications at all including only one of the six nicotine cases and none of the nine “clean” controls, b) these are cell cultures considerably removed from the direct presence of these medications and c) the case and control profiling of lymphoblast cultures derived from 16 panic disorder probands, some of whom were not on medications, shows similar results. Fourth, these results are not corrected for multiple comparisons. However, it is noted that many of these syndromes are inter-correlated (Table 2) and the significance of many of these findings would survive any correction. Fifth and finally, it should be noted that the profiled cells are lymphoblasts, not lymphocytes. However, it is noted that others have demonstrated that they have similar gene expression properties (Morello et al. 2004 Arterioscler Thromb Vasc Biol 24(11): 2149-2154).

The microarray and RTPCR data with respect to ELN expression and nicotine dependence was the most interesting for several reasons. First, panic disorder is three-fold more common in those with nicotine dependence and linkage for panic disorder has been linked to the 7q21 locus containing elastin. Second, in previous work, we have investigated the role of this gene in panic disorder and found mildly suggestive evidence of a role for in panic disorder. Third, ELN and AUTS2 (which was associated with current smoking status in this study), both of which were down regulated in the nicotine microarray expression data, each map to 7q21. This suggests the possibility that altered gene expression across the gene region may be differentially affected in nicotine dependence and that role of genetic variability and epigenetic modifications at these linked loci should be explored.

In contrast to initial expectations, 5HTT expression by itself was not associated with any syndrome. There was a modest effect of 5HTT expression levels when combined with information from ELN expression with respect to nicotine dependence. This is not surprising since nicotine dependence and major depression have a common overlapping genetic substrate (Levinson et al. 2003 Am J Med Genet 119B(1): 118-30) and the role for altered serotonergic activity in both major depression and nicotine dependence is strong. Still, it is mildly surprising that 5HTT levels did not significantly correlate with lifetime symptom counts for depression. However, since some of the subjects who were depressed probably were being treated with drugs that affect 5HTT function, it is possible that the presence of these antidepressants confounded the subsequent expression in the lymphoblast cell lines.

The distinctive association patterns of AUTS2 and CAPN2 gene expression in both substance use and ASPD is particularly intriguing. ASPD and substance dependence in general have strongly overlapping genetic and environmental diatheses. If replicated, the strong association of AUTS2 expression with both cannabis use and ASPD, but CAPN2 only with cannabis, dependence may allow the beginning of the dissection of these intertwined etiologies.

A critical question is why gene expression in cell lines derived from the periphery is correlated with processes more associated with CNS function. One reason may be that their expression may be affected by the same genetic and environmental factors as their counterparts in the CNS. On the face, this proposition may seem ill-founded. But it may well be that physiological triggers of abnormal behavior such as low thyroid hormone or high cortisol levels that can be produced by somatic illness or environmental stress may leave the same indelible signature on gene transcription in the periphery as it does on behavior emanating from CNS processing. Furthermore, similar results are noted from case and control profiling of subjects with panic disorder.

This caveat is particularly important because other groups have attempted to develop expression profile signatures of schizophrenia (Glatt et al. 2005 Proc Natl Acad Sci USA 102(43): 15533-8). Unfortunately, the profiles developed by the groups largely do not overlap. However, since one group used whole blood RNA while the other group used whole blood lymphocyte RNA, both of which contain RNA from mixed cell populations, it is quite possible that the failure to generate similar profiles has resulted from methodological differences. Since the current study uses RNA derived from a single precursor cell type, B-lymphocytes, it may well generate a more robust signature for certain illnesses.

In summary, the inventors report that expression profiling of lymphoblast cell lines demonstrates significant relationships of gene expression with lifetime symptom counts of major depression and antisocial personality disorder, as well as nicotine, cannabis and alcohol dependence. Since these cell lines strongly mimic the expression of their corresponding native B-lymphocytes, the expression profiling of MRNA expression provide a mechanism for the development of laboratory diagnostic algorithms for the common mental disorders.

### EXAMPLE 2

### Panic Disorder

Panic disorder (PD) affects 3.5% of the US population. Its hallmark is the occurrence of discrete episodes of fear, accompanied by various psychological and physiological symptoms including increased heart rate, shortness of breath and diaphoresis. In efforts to define genetic factors associated with increased vulnerability to PD, several genome-wide linkage studies and a large number of candidate gene studies have been conducted. Despite these efforts, specific genetic variation containing altered vulnerability to PD has not been unambiguously identified. This lack of success to date, suggests that alternative approaches to identify genetic factors associated with altered vulnerability to PD may be necessary.

One such alternative approach is transcriptional profiling. Unfortunately, the ideal material to study, brain tissue, is difficult to obtain in quantities and qualities necessary for scientific study. Therefore, if alternative sources of RNA could be identified, they could conceivably contribute to the understanding of the biology of PD, even if they are not derived from the CNS.

One such tissue source may be lymphocytes and their derived lymphoblast cells lines. In an increasing number of studies, investigators have demonstrated that the transcriptional signatures found in either lymphoblasts or lymphoblast cell lines are predictive of complex “medical” illnesses including hyperlipidemia, hypertension and arthritis. Since nearly 82% of the genes expressed in the CNS are expressed in peripheral blood and the expression of genes in peripheral blood is affected by “environmental conditions,” if environmental or gene-environmental interactions are important in the etiology of a disorder, then it is possible that peripheral blood cells could serve as a biosensor or sentinel of disease vulnerability. Furthermore, since lymphoblasts retain the transcriptional profile of the lymphoblasts from which they are derived, it may be possible to study a portion of the biology of psychiatric syndromes by examining the lymphoblast cell lines already residing in repositories.

The present inventor' laboratory routinely uses these cultures to examine the role of genetic variation and methylation on gene transcription. Therefore, to expand these studies and to discern whether lymphoblasts retain transcriptional signatures of complex behavioral illnesses, the inventors performed genome wide transcriptional profiling on 16 independent probands and 17 psychiatrically well controls.

### MATERIALS AND METHODS

The 16 “case” lymphoblast cell lines (10 female, 6 male) used for this study were derived from independent, unrelated probands who participated in previously described genetic studies of PD (Crowe et al. 2001, American Journal of Medical Genetics 105(1):105-9). The 17 “control” lymphoblast lines (11 female, 6 male) were derived from participants in the Iowa Adoption Studies who do not have a history of significant psychiatric illness including major depression, panic disorder or substance use. All lines were prepared using standard EBV transfection methods (Ginns et al., 1996, Nat Genet 12(4):431-5) and grown using standard bovine serum based growth media supplemented with L-glutamine and penicillin/streptomycin. The lymphoblast lines from the PD probands are all second passage (previously stored in liquid nitrogen) while the lines from the control subjects are all first passage. Media was changed for each of the cell lines 24 hours prior to extraction.

The overall design and methodology of the Iowa Adoption Studies and how the demographic and clinical cohorts were gathered is described in detail above. All clinical data used in this study were derived from structured interviews by trained research assistants using the Structured Assessment for the Genetic Studies of Alcoholism (SSAGA). The well controls have been interviewed on at least three separate occasions and on each time have denied histories of major behavioral illness including substance use disorders and panic attacks.

The methods describing the ascertainment and clinical characterization of the PD probands has also been described previously (Crowe et al. 2001, American Journal of Medical Genetics 105(1):105-9). Briefly, a staff psychiatrist or a senior psychiatry resident interviewed the subjects with the Structured Clinical Interview for DSM-III-R (SCID). The interviewer wrote a narrative summary and obtained records of psychiatric treatment. Using this clinical material, two independent diagnosticians made best-estimate diagnoses based on DSM-III-R criteria and resolved differences by consensus agreement. The procedures and methods for both the Iowa Adoption Studies and the Panic Disorder studies were approved by the University of Iowa Institutional Review Board for Human Subjects.

RNA was extracted from the cell lines using standard procedures using an Invitrogen™ RNA purification kit (Invitrogen™, Carlsbad, Calif.) according to the instructions provided by the manufacturer and stored in Ambion® RNA storage solution at −80°. All samples were examined spectrophotometrically to ensure quality with a subset of the samples being further analyzed using an Agilent Bioanalyzer.

Microarray analysis of the RNA extracted from the lymphoblast cell lines was conducted as previously described (Foltz et al. 2006, Cancer Res 66(13):6665-74). Briefly, total RNA (2 μg) was reverse-transcribed with the Chemiluminescent RT-IVT Labeling Kit (Applied Biosystems, Foster City, Calif.) and was hybridized to the ABI 1700 Human Genome Expression Microarray containing 29,098 gene-specific 60-mer oligonucleotide probes per manufacturer's protocol. Data were quantile normalized and a t-test was applied to each gene for statistical significance. Storey q-value method was then used to assess false discovery rate to determine the number of genes differentially expressed in 80% of the cell lines (Storey et al. 2003, Methods Mol Biol 224:149-57). The significantly up-regulated and down-regulated gene lists represent those genes whose expression was arithmetically changed in the same direction (i.e., up or down) in each of the cases and significantly changed as compared to the controls for all PD subjects (n=16), or just male (n=6) or female (n=10). The relationship (or clustering analysis) of the differentially expressed genes to known pathways was analyzed using the Panther analysis subroutine and default settings (Applied Biosystems, see world-wide-web at pantherdb.org) using the binomial test as described by Cho and Campbell (Trends Genet 16(9):409-15).

The inventors then tested the validity the expression profiles at five loci using RTPCR. To select the best candidates for RTPCR, the inventors first examined the expression of the transcript in hemopoetic tissue using the GeneCards® website (see world-wide-web at genecards.org). Then from those differentially expressed genes described above, the inventors picked the transcripts for TCP1 (t-complex 1, 6q25), GYS2 (glycogen synthase 2, 12p12.2), DERL3 (Der1-like domain family, member 3, 22q11) and KIAA 1183 (19q13) for RT-PCR examination using their differential regulation combined with their expression levels in hemopoetic tissues as the basis for our selection. The fifth transcript, COMT (catechol O methyltranferase, 22q11), picked for RT-PCR analysis was selected because of the great interest in this locus. RT-PCR was conducted as described above. First, 5 μg aliquots of total RNA samples from all 33 cell lines were converted to cDNA using an Applied Biosystems cDNA Archiving Kit (Applied Biosystems, Foster City, Calif.) using the same set of solutions to minimize conversion variability. Unfortunately, the tube caps for two control samples leaked rendering their cDNAs samples useless. The resulting 31 cDNA solutions were then diluted and aliquoted into master plates for robotic liquid handling. Quantification of gene expression for these 2 transcripts was then performed using Taqman® Universal Master Mix Reagents (PE Biosystems, Branchburg, N.J.) in combination with Assays-on-Demand® primer/probe sets specific for the TCP1 and COMT loci (Hs00830779, Hs00241349). Relative levels of these RNAs were determined by the comparative CT method using LDHA (lactase dehydrogenase A) and GAPDH (glyceraldehyde-3-phosphate dehydrogenase) as normalizing controls. Z-scores for each RT-PCR run were calculated as per Fleiss (1981) and all runs were performed in duplicate on an ABI 7900HT Sequence Detection System using 12.5 ng of cDNA. The Pearson's Correlation for duplicate samples for each primer probe set was greater than>99%. The correlation coefficient for the Z-scores of LDHA and GAPDH was 0.91. ANOVA analysis was conducted using JMP (version 5.1; SAS Institute, Cary, N.C., USA).

### RESULTS

Table 5 describes the age, sex and medications of each PD subject. Two subjects were not on any medications. The remainder were on a variety of antidepressants, mainly tricyclics, and/or benzodiazepines. The control subjects were not on any psychiatric medications. The female case and control subjects did differ significantly at the age of phlebotomy (42±9 years vs. 44±9 years). However, the male cases were significant younger than the male controls (33±7 years vs. 49±9 years; p<0.006).

TABLE 5
Characteristics of Panic Disorder Pro bands

| Proband | Sex | Age | Medications |
|---|---|---|---|
| 1 | male | 21 | none |
| 2 | male | 35 | none |
| 3 | female | 40 | imipramine |
| 4 | female | 45 | imipramine |
| 5 | female | 31 | alprazolam |
| 6 | female | 41 | alprazolam, desipramine, |
| | | | clonezapam, perphenazine |
| 7 | female | 58 | lorazepam |
| 8 | female | 33 | chlorazepate, amitriptyline |
| 9 | female | 54 | perphenazine, amitriptyline |
| 10 | male | 39 | imipramine |
| 11 | female | 33 | imipramine |
| 12 | female | 38 | lorazepam |
| 13 | male | 35 | carbamezapine, sertraline |
| 14 | male | 38 | fluoxetine |
| 15 | female | 47 | diazepam |
| 16 | male | 32 | sertraline |

Tables 6A and 6B list the thirty most up-regulated and down-regulated genes for all subjects overall. It should be noted that this list is composed of those transcripts whose relative expression was increased or decreased, respectively, in every proband and significantly increased overall as compared to the control values. Overall, the expression of 2469 transcripts was arithmetically increased in every PD cell line and significantly increased overall. Conversely, 354 transcripts were arithmetically decreased in every PD cell line in every PD cell line and significantly decreased overall.

TABLE 6A
The 30 most up-regulated transcripts.

| Gene Symbol | Cytoband | Gene Name | female fold Δ | q-value | male fold Δ | q-value |
|---|---|---|---|---|---|---|
| GYS2 | 12p12.2 | glycogen synthase 2 (liver) | 12.36 | <0.002 | 12.43 | <0.01 |
| IL19 | 1q32.2 | interleukin 19 | 11.39 | <0.003 | 12.64 | <0.005 |
| TCP1 | 6q25-q27 | t-complex 1 | 11.27 | <0.005 | 4.39 | <0.14 |
| LOC55908 | 19p13.2 | carcinoma-associated gene TD26 | 11.18 | <0.005 | 11.38 | <0.03 |
| MMP23A&B | 1p36.3 | matrix metalloprotease 23A and 23B | 10.89 | <0.003 | 11.36 | <0.04 |
| CAV2 | 7q31.1 | caveolin 2 | 10.87 | <0.002 | 12.57 | <0.02 |
| DLEU2 | 13q14.3 | deleted in lymphocytic leukemia, 2 | 10.76 | <0.005 | 11.32 | <0.02 |
| CSMD2 | 1p34.3 | CUB and Sushi multiple domains 2 | 10.72 | <0.005 | 10.94 | <0.02 |
| CACNA1A | 19p13.2 | calcium channel, P/Q type, alpha IA subunit | 10.71 | <0.005 | 10.78 | <0.02 |
| ARMC2 | 6q21 | armadillo repeat containing 2 | 10.69 | <0.03 | 9.86 | <0.08 |
| DERL3 | 22q11.23 | chromosome 22 open reading frame 14 | 10.38 | <0.003 | 10.43 | <0.001 |
| TCP10L;C21orf77 | 21q22.11 | t-complex 10 (mouse)-like; ORF 77 | 10.33 | <0.005 | 11.90 | <0.01 |
| TSPAN1 | 1p134.1 | tetraspan 1 | 9.89 | <0.005 | 10.93 | <0.01 |
| CYP2R1 | 11p15.2 | cytochrome P450, family 2, subfamily R | 9.86 | <0.004 | 10.16 | <0.02 |
| | | subfamily R, polypeptide 1 | | | | |
| MGC26979 | 8q22.1 | hypothetical protein MGC26979 | 9.82 | <0.004 | 0.40 | <0.02 |
| PPP2R2C | 4p16.1 | protein phosphatase 2, regulatory subunit B | 9.82 | <0.004 | 9.15 | <0.001 |
| | | (PR 52), gamma isoform | | | | |
| THAP9 | 4q21.3 | THAP domain containing 9 | 9.81 | <0.005 | 10.60 | <0.01 |
| UBR2 | 6p21.1 | chromosome 6 open reading frame 133 | 9.67 | <0.008 | 10.10 | <0.02 |
| LZTFL1 | 3p21.3 | leucine zipper transcription factor-like 1 | 9.55 | <0.005 | 9.85 | <0.02 |
| GOPC | 6q21 | golgi associated PDZ and | 9.51 | <0.006 | 9.33 | <0.03 |
| | | coiled-coil motif containin<0.001 g | | | | |
| HIST1H4A | 6p21.3 | histone 1, H4a | 9.36 | <0.005 | 9.78 | <0.02 |
| VWF | 12p13.3 | von Willebrand factor | 9.35 | <0.003 | 9.10 | <0.08 |
| DEFB118 | 20q11.1 | defensin, beta 118 | 9.34 | <0.005 | 9.08 | <0.03 |
| KIAA1181 | 5q35.2 | KLAA1181 protein | 9.31 | <0.006 | 9.44 | <0.02 |
| TNPO2 | 19p13.2 | transportin 2 | 9.19 | <0.001 | 8.76 | <0.002 |
| ZNF572 | 8q24.13 | zinc finger protein 572 | 9.19 | <0.03 | 8.70 | <0.08 |
| CACNB2 | 10p12 | calcium channel beta 2 subunit | 9.16 | <0.005 | 9.96 | <0.008 |
| SSH2 | 17q11.2 | slingshot homolog 2 (Drosophila) | 9.16 | <0.006 | 9.07 | <0.03 |
| AGXT2 | 5p13 | alanine-glyoxylate aminotransferase 2 | 9.09 | <0.004 | 9.56 | <0.02 |
| C13orf7 | 13q22.2 | chromosome 13 open reading frame 7 | 9.00 | <0.004 | 9.10 | <0.02 |

TABLE 6B
The 30 most down-regulated transcripts.

| Gene Symbol | Cytoband | Gene Name | female fold Δ | q-value | male fold Δ | q-value |
|---|---|---|---|---|---|---|
| TDG | 12q24.1 | thymine-D glycosylase | −0.99 | <0.007 | −0.39 | <0.02 |
| CBX1 | 17q | chromobox homolog 1 | −0.99 | <0.003 | −0.59 | <0.05 |
| FLJ13984 | 2q31.1 | hypothetical protein FLJ13984 | −0.99 | <0.009 | −0.87 | <0.03 |
| IREB2 | 15q24.1 | iron-responsive element binding protein 2 | −0.98 | <0.004 | −0.98 | <0.006 |
| SAP130 | 2q21.1 | mSin3A-associated protein 130 | −0.98 | <0.007 | −0.76 | <0.10 |
| ESPL1 | 12q | extra spindle poles like 1 (S. cerevisiae) | −0.97 | <0.008 | −0.72 | <0.14 |
| REV3L | 6q21 | REV3-like, catalytic subunit of D polymerase | −0.97 | <0.007 | −0.73 | <0.10 |
| VEGFB | 11q13 | vascular endothelial growth factor B | −0.96 | <0.02 | −0.76 | <0.09 |
| LOC96610;BMS1L | 22q11.22 | hypothetical protein similar to KIAA0187 | −0.96 | <0.005 | −0.42 | <0.14 |
| PP3856 | 8q24.3 | similar to CG3714 gene product | −0.94 | <0.003 | −0.79 | <0.007 |
| SRI | 7q21.1 | sorcin | −0.94 | <0.02 | −0.97 | <0.08 |
| CACNG6 | 19q13.4 | calcium channel, voltage-dependent, γ-6 subunit | −0.94 | <0.007 | −0.58 | <0.10 |
| EPHA2 | 1p136 | EphA2 | −0.94 | <0.01 | −0.39 | <0.05 |
| HAX1 | 1q22 | HS1 binding protein | −0.93 | <0.006 | −0.75 | <0.03 |
| STK23 | Xq28 | serine/threonine kinase 23 | −0.93 | <0.006 | −0.65 | <0.006 |
| TRNT1 | | tR nucleotidyl transferase, CCA-adding, 1 | −0.92 | <0.008 | −0.82 | <0.07 |
| PGM2 | 4p14 | phosphoglucomutase 2 | −0.90 | <0.005 | −0.86 | <0.03 |
| ATPAF2 | | ATP synthase mitochondrial F1 CAF2 | −0.90 | <0.01 | −0.77 | <0.13 |
| Cl4orf133 | 14q24.3-q31 | chromosome 14 open reading frame 133 | −0.90 | <0.005 | −0.92 | <0.09 |
| GPR54 | 19p13.3 G | protein-coupled receptor 54 | −0.90 | <0.009 | −0.69 | <0.05 |
| OR1D2 | 17p13-p12 | olfactory receptor, family 1, | −0.90 | <0.03 | −0.83 | <0.06 |
| | | subfamily D, member 2 | | | | |
| TAOK3 | 12q | STE20-like kinase | −0.89 | <0.006 | −0.45 | <0.11 |
| PSCDBP | 2q11.2 | pleckstrin homology, Sec7 and | −0.89 | <0.005 | −0.67 | <0.06 |
| | | coiled-coil domains, binding protein | | | | |
| DYRK2 | 12q14.3 | dual-specificity tyrosine-(Y)- | −0.89 | <0.04 | −0.68 | <0.04 |
| | | phosphorylation regulated kinase 2 | | | | |
| CARD9 | 9q34.3 | caspase recruitment domain family, no. 9 | −0.89 | <0.02 | −0.95 | <0.02 |
| NDEL1 | 17p13.1 | nudE nuclear distribution gene E homolog | −0.89 | <0.02 | −0.88 | <0.13 |
| INPP1 | 2q32 | inositol polyphosphate-1-phosphatase | −0.88 | <0.02 | −0.44 | <0.13 |
| SLC7A8 | 4q11.2 | solute carrier family 7, member 8 | −0.88 | <0.01 | −0.93 | <0.03 |
| SRPK2 | 7q22-q31.1 | SFRS protein kinase 2 | −0.87 | <0.007 | −0.54 | <0.05 |
| IGLL1 | 22q11.23 | immunoglobulin lambda-like polypeptide 1 | −0.86 | <0.03 | −0.39 | <0.05 |

Gene name is per GenBank.
The extent of up-regulation is expressed as the “fold” of the net change for each sex. Where known, the function of the gene according to Panther analytic subroutine is given.
The calculation of q-values was performed as described in the methods section.

Since many behavioral disorders may have unique sex specific biological diatheses, we also conducted sex specific analyses. These analyses demonstrated that in female PD cell lines, the expression of a further 67 transcripts was arithmetically increased in every line and significantly increased overall, as compared to female control lines, and a further 332 transcripts was arithmetically decreased in every cell line and significantly decreased overall. The top 30 most up-regulated and down-regulated genes are listed in Table 7A and 7B.

TABLE 7A
The 30 transcripts most significantly up-regulated in females only.

| Gene Symbol | Cytoband | Gene Name | female fold Δ | q-value | male fold Δ |
|---|---|---|---|---|---|
| TM7SF3 | 12q11-q12 | transmembrane 7 superfamily no. 3 | 5.78 | <0.03 | −1.43 |
| SYNGR3 | 16p13 | syptogyrin 3 | 3.35 | <0.03 | −1.28 |
| EFNB3 | 17p13.1-ephrin-B3 | | 2.04 | <0.01 | 0.88 |
| FLJ33674 | 3p25.3 | hypothetical protein FLJ33674 | 2.02 | <0.005 | 0.93 |
| PFN2 | 3q25.1-q25.2 | profilin 2 | 2.00 | <0.04 | 0.66 |
| TNFSF11 | 13q14 | tumor necrosis factor superfamily, member 11 | 1.80 | <0.005 | 0.93 |
| LRRC35 | 11q23.3 | hypothetical protein MGC10233 | 1.75 | <0.02 | 0.63 |
| PLXDC2 | 10p12.33 | plexin domain containing 2 | 1.72 | <0.03 | 0.89 |
| LY96 | 8q13.3 | lymphocyte antigen 96 | 1.64 | <0.008 | 0.79 |
| TFF1 | 21q22.3 | trefoil factor 1 | 1.60 | <0.01 | 0.92 |
| CHRNB2 | 1q21.3 | cholinergic receptor, nicotinic, beta 2 | 1.57 | <0.01 | 0.91 |
| MGC3771 | 16p13.13 | hypothetical protein MGC3771 | 1.56 | <0.01 | 0.99 |
| EFNA5 | 5q21 | ephrin-A5 | 1.49 | <0.005 | 0.95 |
| PRKAG3 | 2q36.1 | protein kinase, AMP-activated, | 1.46 | <0.007 | 0.90 |
| | | gamma 3 non-catalytic subunit | | | |
| FLII | 17p11.2 | flightless I homolog (Drosophila) | 1.46 | <0.009 | 0.83 |
| LRRC16 | 6p22.1 | leucine rich repeat containing 16 | 1.42 | <0.006 | 0.93 |
| MCPH1 | 8p23.1 | hypothetical protein FLJ12847 | 1.39 | <0.03 | 0.75 |
| SREBF2 | 22q13 | sterol regulatory element binding | 1.37 | <0.02 | 0.86 |
| | | transcription factor 2 | | | |
| RFPL2 | 22q12.3 | ret finger protein-like 2 | 1.36 | <0.005 | 0.76 |
| FBXO3 | 11p13 | F-box protein 3 | 1.35 | <0.01 | 0.97 |
| LMNA | 1q21.2-q21.3 | lamin A/C | 1.35 | <0.05 | 0.93 |
| DJ122O8.2 | 6q14.2-q16.1 | hypothetical protein dJ122O8.2 | 1.35 | <0.01 | 0.72 |
| RAB38 | 11q14 | RAB38, member RAS oncogenes | 1.31 | <0.007 | 0.93 |
| TGFB1I1 | 16p11.2 | transforming growth factor beta 1 | 1.31 | <0.03 | 0.91 |
| | | induced transcript 1 | | | |
| IMMP2L | 7q31 | IMP2 inner mitochondrial membrane protease | 1.31 | <0.01 | 0.91 |
| FLJ38482 | 4q32.3 | hypothetical protein FLJ38482 | 1.31 | <0.003 | 0.97 |
| C20orf13 | 20p12.1 | chromosome 20 ORF 13 | 1.30 | <0.02 | 0.95 |
| YPEL1 | 22q11.2 | yippee-like 1 (Drosophila) | 1.30 | <0.001 | 0.86 |
| UPB1 | 22q11.2 | ureidopropiose, beta | 1.28 | <0.02 | 0.99 |

Gene name is per GenBank.
The extent of up-regulation is expressed as the “fold” of the net change for each sex. Where known, the function of the gene according to Panther analytic subroutine is given.
The calculation of q-values was performed as described in the methods section.

TABLE 7B
The 30 transcripts most significantly down-regulated in females only.

| Gene Symbol | Cytoband | Gene Name | female fold Δ | q-value | male fold Δ |
|---|---|---|---|---|---|
| CDT1 | 16q24.3 | D replication factor | −1.00 | <0.005 | −1.48 |
| LOC93349 | 2q37.1 | hypothetical protein BC004921 | −1.00 | <0.006 | −1.08 |
| MLLT10 | 10p12 | myeloid/lymphoid or mixed-lineage | −1.00 | <0.03 | −1.96 |
| | | leukemia (trithorax homolog) | | | |
| GMRP-1 | 11p15.3 | K + channel tetramerization protein | −1.00 | <0.007 | −1.22 |
| NRG2 | q23-q33 | neuregulin 2 | −0.99 | <0.003 | −1.31 |
| STX1B2 | 16p12-p11 | syntaxin 1B2 | −0.99 | <0.01 | −2.02 |
| RPL14 | 3p22-p21.2 | ribosomal protein L14 | −0.99 | <0.03 | −1.48 |
| MGC29649 | 11q12.3 | hypothetical protein MGC29649 | −0.99 | <0.005 | −1.45 |
| SLC16A11 | 17p13.2 | solute carrier family 16, member 11 | −0.98 | <0.005 | −1.01 |
| SCYL1 | 11q13 | SCY1-like 1 (S. cerevisiae) | −0.98 | <0.007 | −1.27 |
| MGC17330 | 22q12.2 | HGFL gene | −0.98 | <0.02 | −1.43 |
| MGC4268 | 2q21 | hypothetical protein MGC4268 | −0.97 | <0.03 | −2.05 |
| C6orf54 | 6q27 | chromosome 6 ORF 54 | −0.97 | <0.004 | −1.29 |
| C10orf125 | 10q26.3 | chromosome 10 ORF 125 | −0.97 | <0.03 | −1.85 |
| GCL | 2p13.3 | germ cell-less homolog 1 | −0.97 | <0.008 | 1.52 |
| CGB7 | 19q13.32 | chorionic gonadotropin, B-peptide 7 | −0.97 | <0.02 | −1.83 |
| LOC90624 | 5q31.1 | hypothetical protein LOC90624 | −0.96 | <0.009 | −1.50 |
| SNX6 | 14q13.1 | sorting nexin 6 | −0.96 | <0.004 | −1.03 |
| CRIP1 | 14q32.33 | cysteine-rich protein 1 (intestil) | −0.96 | <0.004 | −1.36 |
| ARHGEF1 | 19q13.13 | Rho guanine nucleotide | −0.96 | <0.02 | −1.53 |
| | | exchange factor (GEF) 1 | | | |
| ARRDC1 | 9q34.3 | arrestin domain containing 1 | −0.96 | <0.005 | −1.20 |
| HT007 | 11q14.1 | hypothalamus protein HT007 | −0.95 | <0.02 | −1.22 |
| CDKN3 | 14q22 | cyclin-dependent kinase inhibitor 3 | −0.95 | <0.009 | −1.05 |
| GABARAPL1 | 12p13.31 | GABA(A) receptor-associated | −0.95 | <0.008 | −1.99 |
| | | protein like 1 | | | |
| SP1 | 12q13.1 Sp1 | transcription factor | −0.95 | <0.05 | −1.83 |
| MAN2B1 | 19cen-q13.1 | mannosidase, alpha, class 2B, no. 1 | −0.94 | <0.02 | −1.79 |
| NCSTN | 1q22-q23 | nicastrin | −0.94 | <0.02 | −1.13 |
| CSNK1G2 | 19p13.3 | casein kinase 1, gamma 2 | −0.94 | <0.004 | −1.35 |
| KRTAP3-1 | 17q12-q21 | keratin associated protein 3-1 | −0.94 | <0.006 | −1.15 |
| ELMO1 | 7p14.1 | engulfment and cell motility 1 | −0.93 | <0.02 | −3.03 |

Gene name is per GenBank.
The extent of up-regulation is expressed as the “fold” of the net change for each sex. Where known, the function of the gene according to Panther analytic subroutine is given.
The calculation of q-values was performed as described in the methods section.

Conversely, in cell lines from the six male PD probands, the expression of an additional 212 was arithmetically increased in every line and significantly increased overall as compared to the male control lines, and a further 332 transcripts was arithmetically decreased in every cell line. The top 30 most up-regulated and down-regulated of these genes are listed in Table 8A and 8B.

TABLE 8A
The 30 transcripts most significantly up-regulated in males only.

| Gene Symbol | Cytoband | Gene Name | female fold Δ | male fold Δ | q-value |
|---|---|---|---|---|---|
| EBNA1BP2 | 1p35-p33 | EB1 binding protein 2 | 0.77 | 3.22 | <0.05 |
| APG12L | 5q21-q22 | APG12 autophagy 12-like | 0.98 | 3.21 | <0.002 |
| COMT | 22q11.21 | catechol-O-methyltransferase | 0.39 | 2.85 | <0.02 |
| MGC35048 | 16p13.11 | hypothetical protein MGC35048 | 0.52 | 2.62 | <0.02 |
| TNFRSF11A | 18q22.1 | TNF receptor superfamily, member 11a | 0.91 | 2.52 | <0.001 |
| GPR30 | 7p22 | G protein-coupled receptor 30 | 0.82 | 2.44 | <0.009 |
| PLA2G12A | 4q25 | phospholipase A2, group XIIA | 0.99 | 2.35 | <0.004 |
| SFMBT1 | 3p21.31 | Scm-like with four mbt domains 1 | 0.98 | 2.32 | <0.003 |
| DPYSL4 | 10q26 | dihydropyrimidise-like 4 | 0.57 | 2.31 | <0.02 |
| LOC200261 | 20p11.22 | hypothetical protein LOC200261 | 0.52 | 2.11 | <0.006 |
| AKAP13 | 15q24-q25 | A kinase (PRKA) anchor protein 13 | 0.75 | 2.07 | <0.002 |
| ARHGAP26 | 5q31 | Rho GTPase activating protein 26 | 0.75 | 2.05 | <0.005 |
| HSPC196 | 11q12.3 | hypothetical protein HSPC196 | 0.48 | 2.05 | <0.002 |
| CENTB5 | | centaurin, beta 5 | 0.99 | 2.00 | <0.02 |
| MGC13053 | 11q23.3 | hypothetical protein MGC13053 | 0.99 | 1.97 | <0.009 |
| EPST11 | 13q13.3 | epithelial stromal interaction 1 | 0.48 | 1.95 | <0.04 |
| ZNF154 | 19q13.4 | zinc finger protein 154 (pHZ-92) | 0.70 | 1.89 | <0.02 |
| KCNK6 | 19q13.1 | potassium channel, subfamily K, | 0.76 | 1.87 | <0.009 |
| | | member 6 | | | |
| ENC1 | 5q12-q13.3 | ectodermal-neural cortex | 0.51 | 1.84 | <0.03 |
| FAM63A | 1q21.3 | hypothetical protein FLJ11280 | 0.53 | 1.84 | <0.02 |
| C7orf26 | 7p22.1 | chromosome 7 ORF 26 | 0.46 | 1.83 | <0.02 |
| HECTD1 | 14q12 | HECT domain containing 1 | 0.21 | 1.83 | <0.02 |
| HT001 | 3q22.1 | HT001 protein | 1.00 | 1.83 | <0.02 |
| TTC12 | 11q23.2 | tetratricopeptide repeat domain 12 | 0.91 | 1.82 | <0.004 |
| COPE | 19p13.11 | coatomer protein complex, epsilon | 0.90 | 1.81 | <0.005 |
| PINK | 11p36 | PTEN induced putative kinase1 | 0.75 | 1.81 | <0.006 |
| GPR109A&B | 12q24.31 | G protein-coupled receptor 109A&B | 0.88 | 1.78 | <0.001 |
| SLC39A7 | 6p21.3 | solute carrier family 39, member 7 | 0.99 | 1.78 | <0.001 |
| HDAC8 | Xq13 | histone deacetylase 8 | 0.97 | 1.76 | <0.004 |
| DICER1 | 14q32.2 | Dicer1, Dcr-1 homolog | 0.52 | 1.75 | <0.02 |

Gene name is per GenBank.
The extent of up-regulation is expressed as the “fold” of the net change for each sex. Where known, the function of the gene according to Panther analytic subroutine is given.
The calculation of q-values was performed as described in the methods section.

TABLE 8B
The 30 transcripts most significantly down-regulated in males only.

| Gene Symbol | Cytoband | Gene Name | female fold Δ | male fold Δ | q-value |
|---|---|---|---|---|---|
| RPL39 | Xq22-q24 | ribosomal protein L39 | −1.24 | −0.99 | <0.02 |
| TEX27 | 6pter-p22.3 | testis expressed sequence 27 | −1.08 | −0.99 | <0.02 |
| SC5DL | 11q23.3 | sterol-C5-desaturase-like | −1.21 | −0.99 | <0.03 |
| FTS | 16q12.2 | fused toes homolog (mouse) | −1.09 | −0.98 | <0.04 |
| CXXC5 | 5q31.3 | CXXC finger 5 | −1.17 | −0.98 | <0.04 |
| TCIRG1 | 11q13.4 | T-cell, immune regulator 1,, | −1.79 | −0.98 | <0.04 |
| NSUN2 | 5p15.32 | hypothetical protein FLJ20303 | −1.11 | −0.98 | <0.02 |
| BRSK2 | 11p15.5 | serine/threonine kinase 29 | −1.43 | −0.98 | <0.05 |
| DNAJB9 | 7q31 | DJ (Hsp4O) homolog, | −1.28 | −0.98 | <0.03 |
| | | subfamily B, member 93 | | | |
| SLC1A7 | 1p32.3 | solute carrier family 1, member 7 | −1.90 | −0.97 | <0.03 |
| IL15RA | 10p15-p14 | interleukin 15 receptor, alpha | −1.31 | −0.96 | <0.03 |
| ABCF3 | 3q27.3 | ATP-binding cassette, sub-family F | −1.06 | −0.95 | <0.03 |
| | | (GCN2O), member 3 | | | |
| COG4 | 16q22.1 | component of golgi complex 4 | −1.47 | −0.95 | <0.02 |
| ADAM19 | 5q32-q33 | a disintegrin and metalloprotease | −1.03 | −0.95 | <0.005 |
| | | domain 19 | | | |
| FAM61A | 19q13.12 | chromosome 19 ORF 13 | −1.20 | −0.95 | <0.02 |
| VPS29 | 12q24 | vacuolar protein sorting 29 (yeast) | −1.05 | −0.95 | <0.04 |
| AK3 | 9p24.1-p24.3 | adenylate kinase 3 like 1 | −1.09 | −0.93 | <0.02 |
| ARV1 | 1q42.2 | likely ortholog of yeast ARV1 | −2.22 | −0.91 | <0.04 |
| GOLGA7 | 8p11.21 | golgi autoantigen, golgin family a7 | −1.04 | −0.90 | <0.03 |
| ARHGAP17 | 16p12.2 | Rho GTPase activating protein 17 | −1.07 | −0.89 | <0.03 |
| TBCE | 1q42.3 | tubulin-specific chaperone e | −1.34 | −0.88 | <0.03 |
| MSL3L1 | Xp22.3 | male-specific lethal 3-like 1 | −1.69 | −0.87 | <0.05 |
| SPON2 | 4p16.3 | spondin 2 | −1.31 | −0.86 | <0.03 |
| C13orf6 | 13q33.3 | chromosome 13 ORF 6 | −1.03 | −0.86 | <0.04 |
| SLC25A1 | 22q11.21 | solute carrier family 25, member 1 | −1.16 | −0.83 | <0.02 |
| FAM26C | 10q24.33 | family with sequence similarity 26, | −1.67 | −0.83 | <0.04 |
| | | member C | | | |
| NADSYN1 | 11q13.2 D | synthetase 1 | −1.29 | −0.83 | <0.05 |
| ATP1B2 | 17p13.1 | ATPase, K + transporting, B2 | −1.03 | −0.83 | <0.02 |
| ZNF213 | 16p13.3 | zinc finger protein 213 | −0.64 | −0.81 | <0.03 |
| PIGQ | 16p13.3 | phosphatidylinositol glycan, class Q | −1.04 | −0.81 | <0.04 |

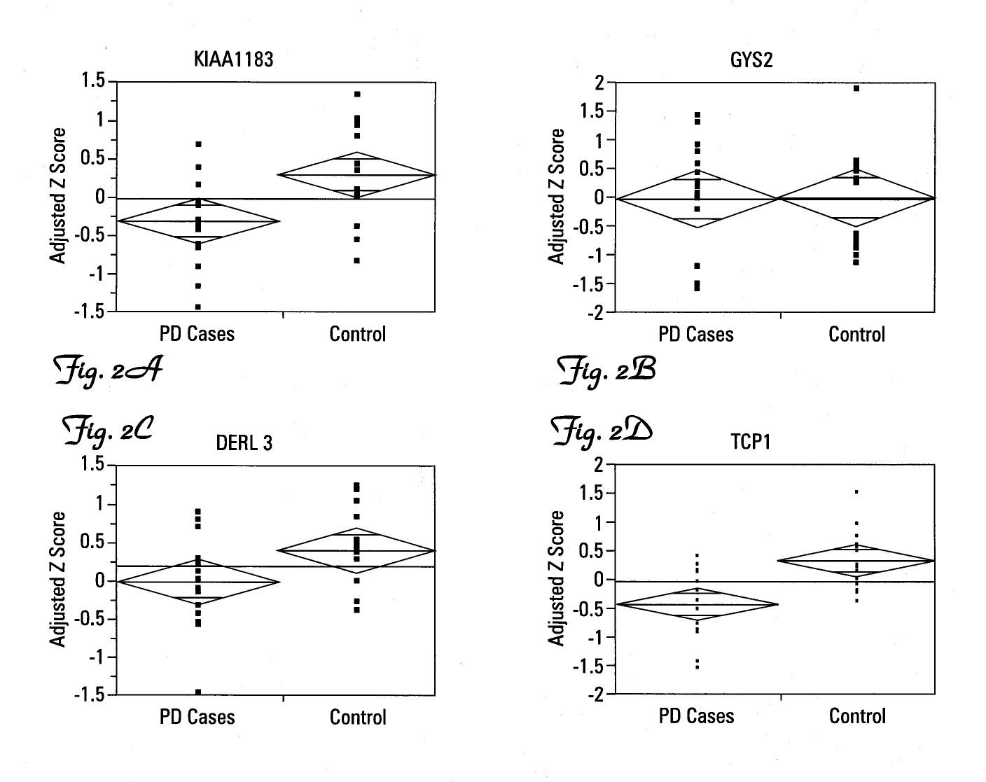

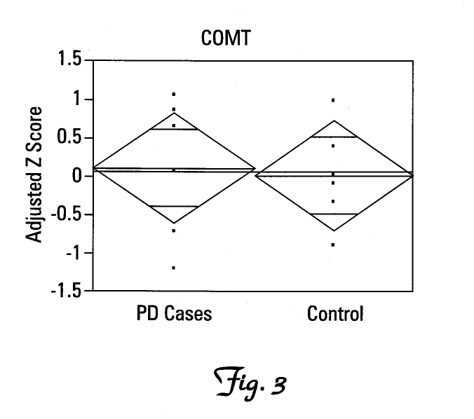

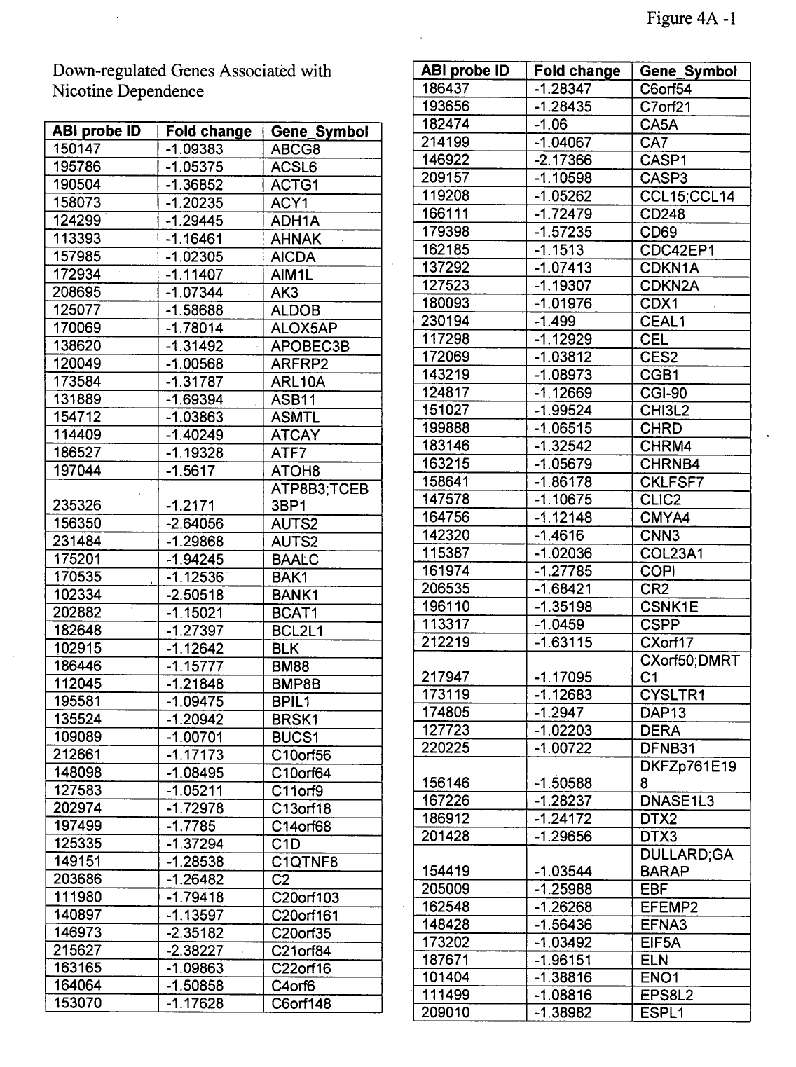

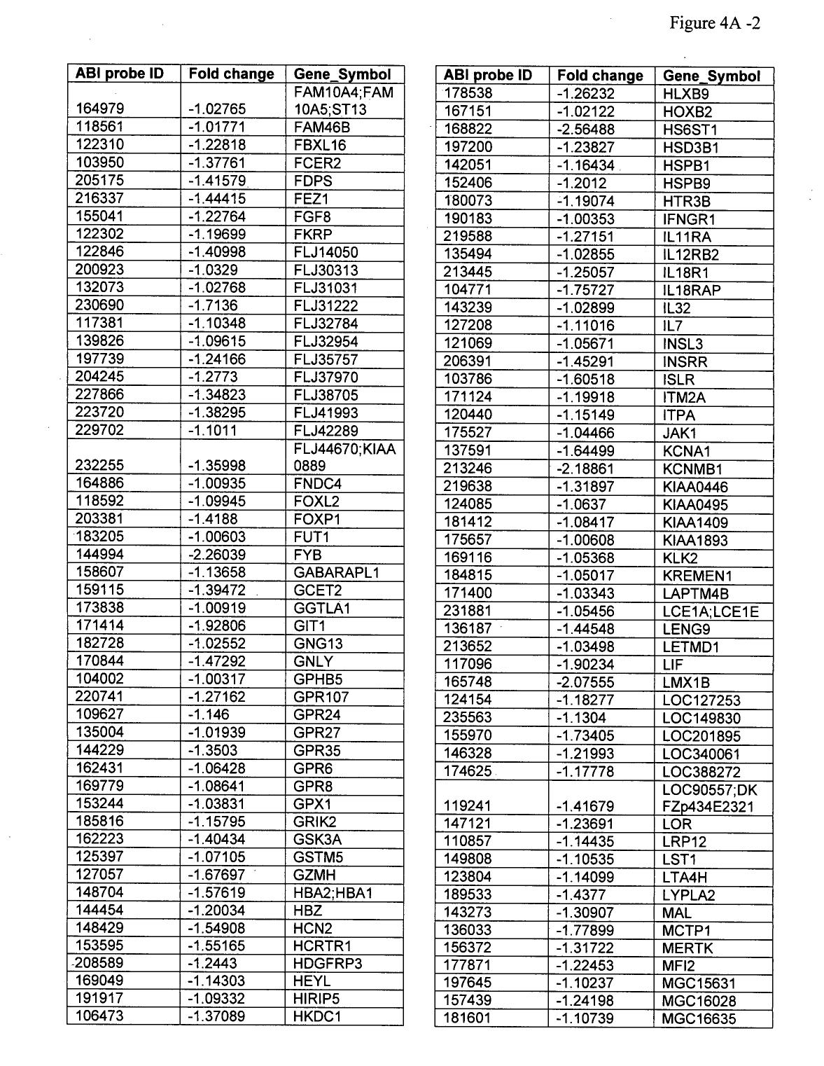

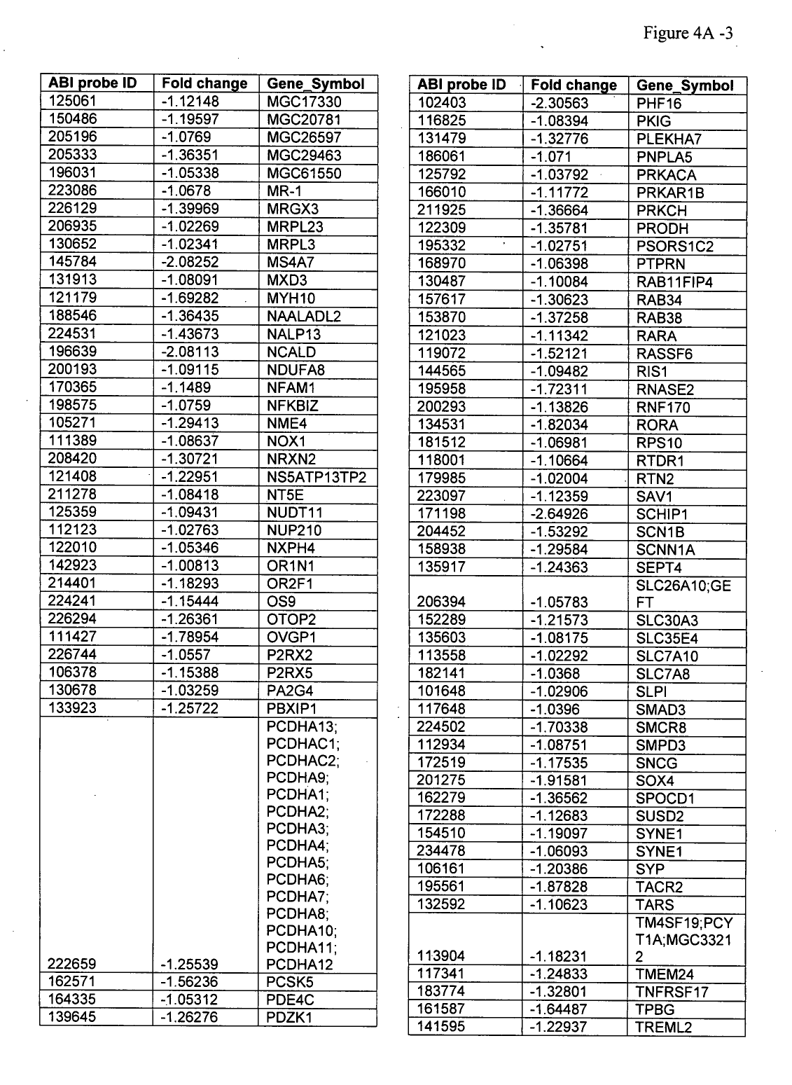

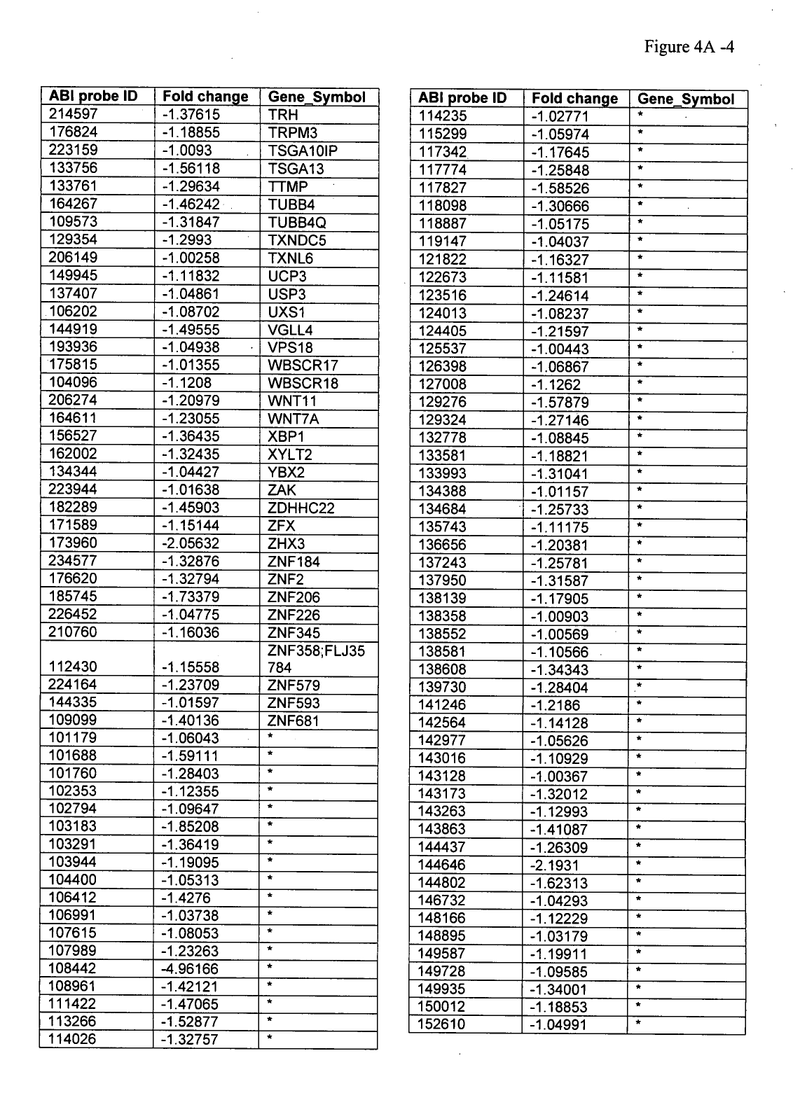

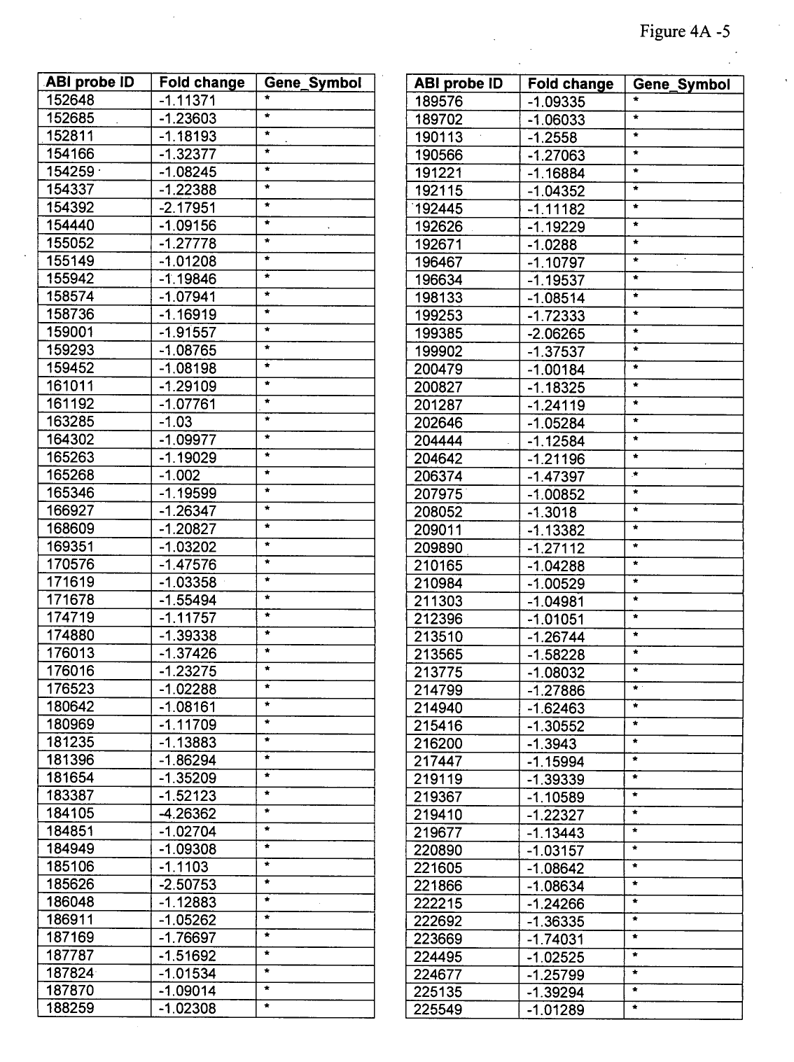

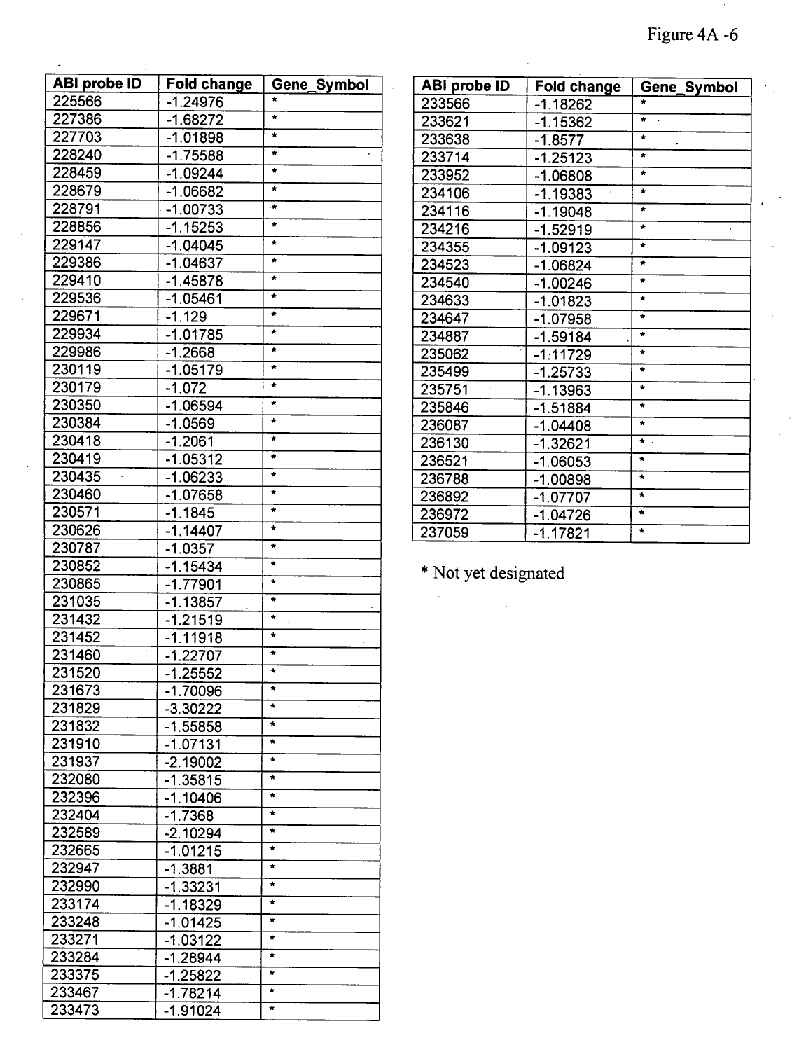

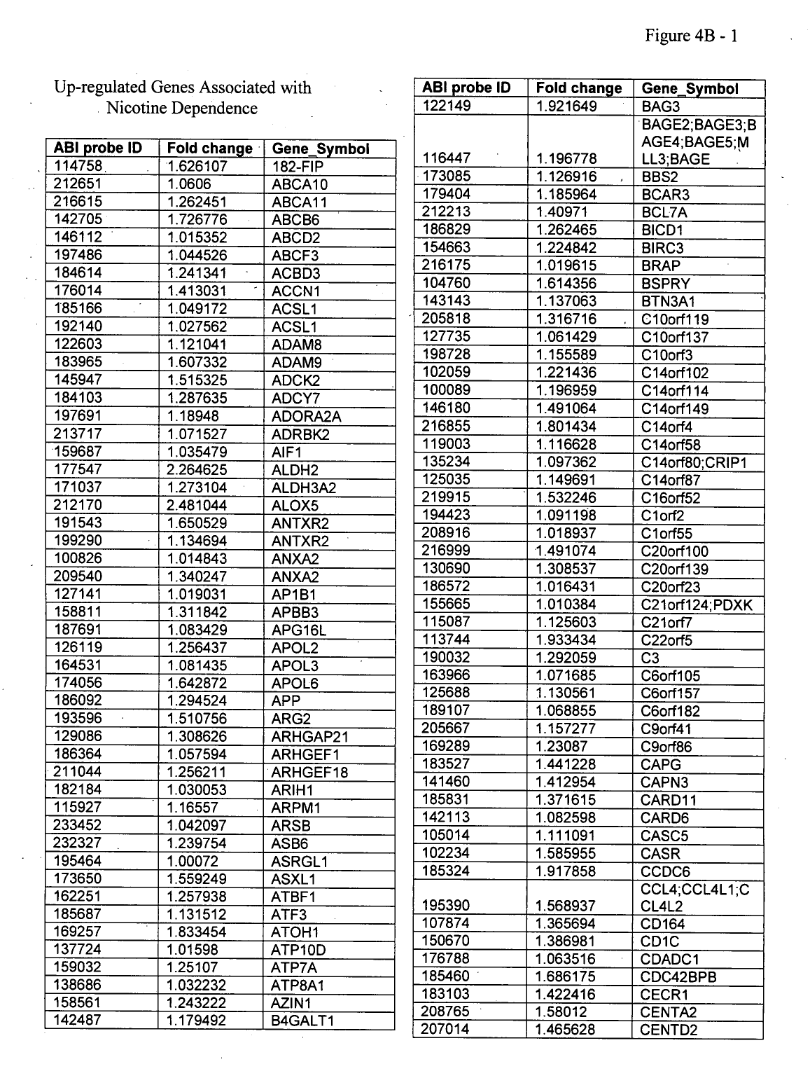

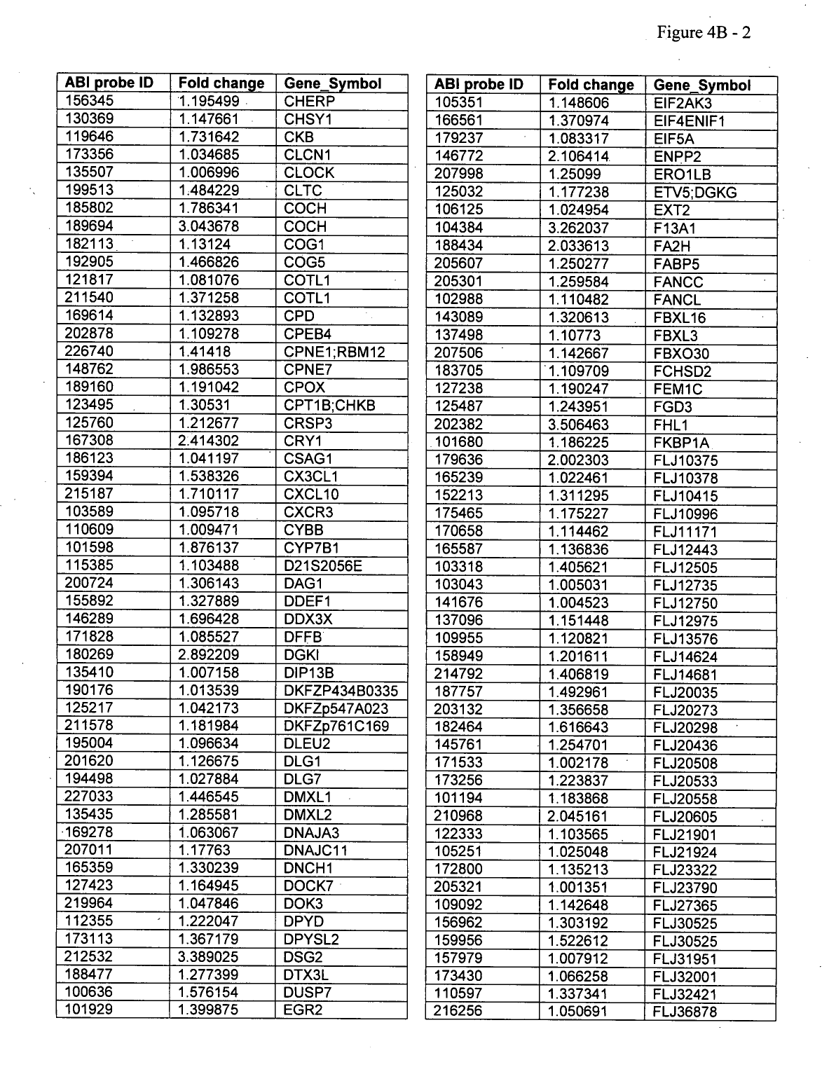

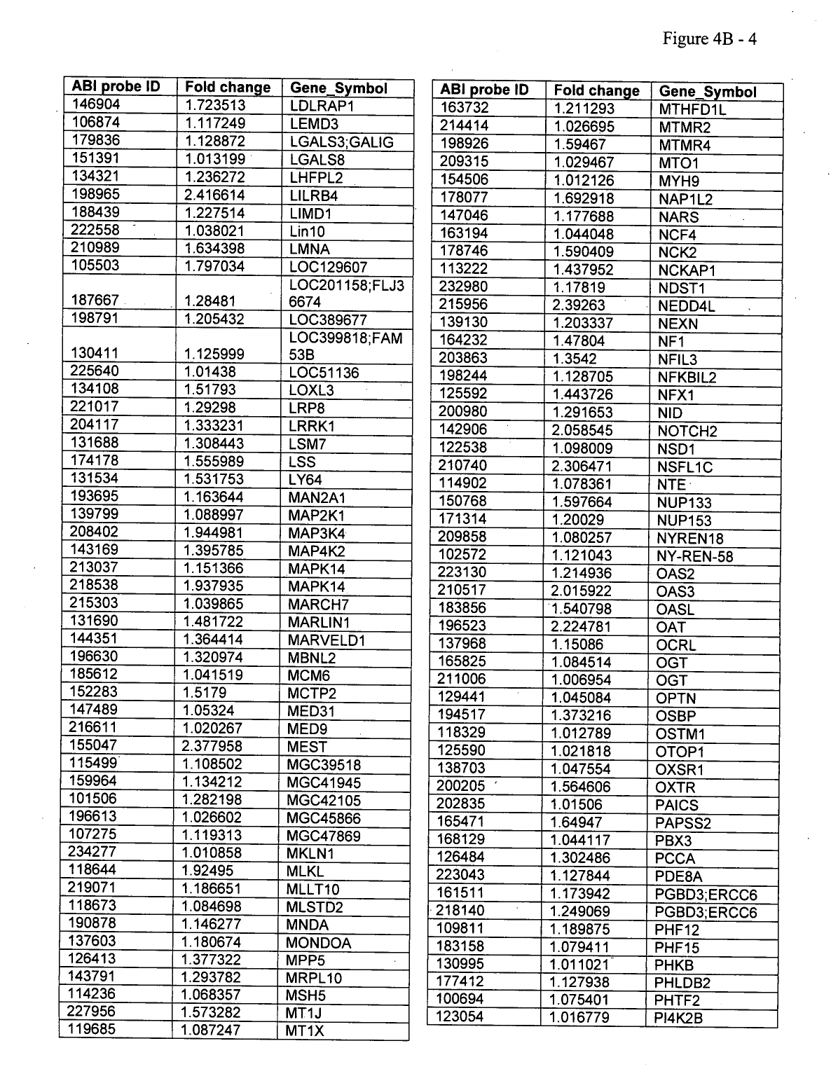

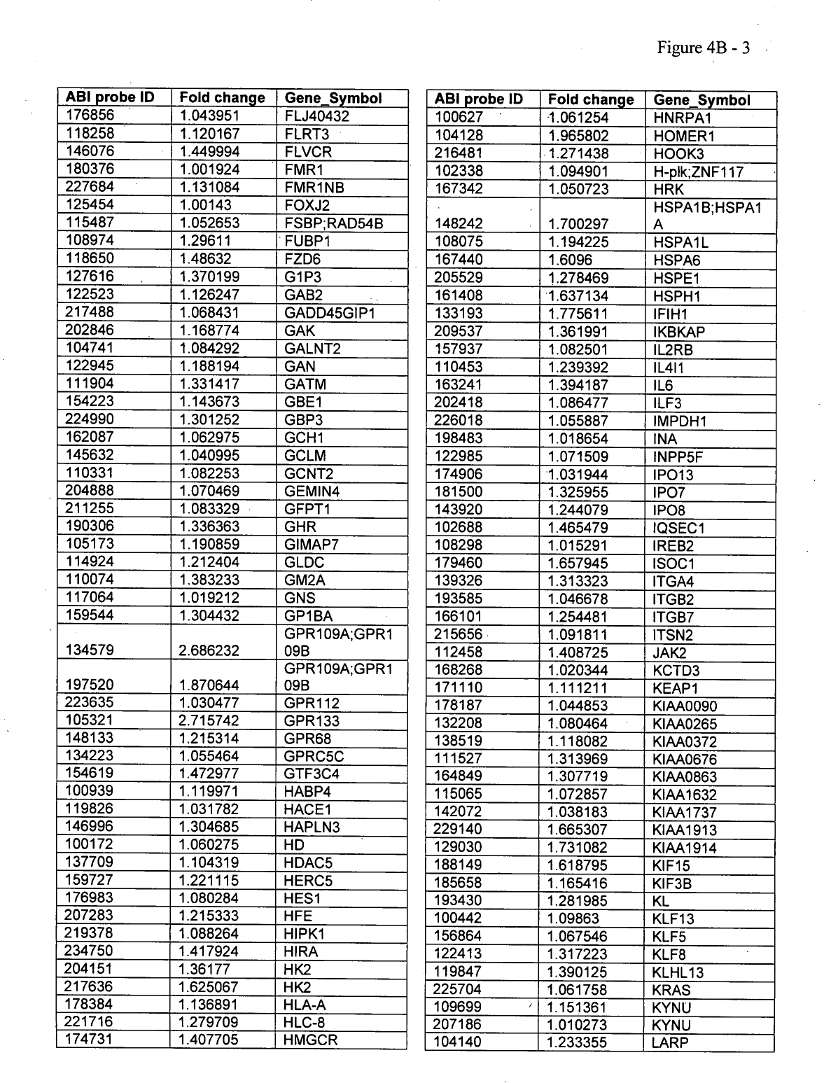

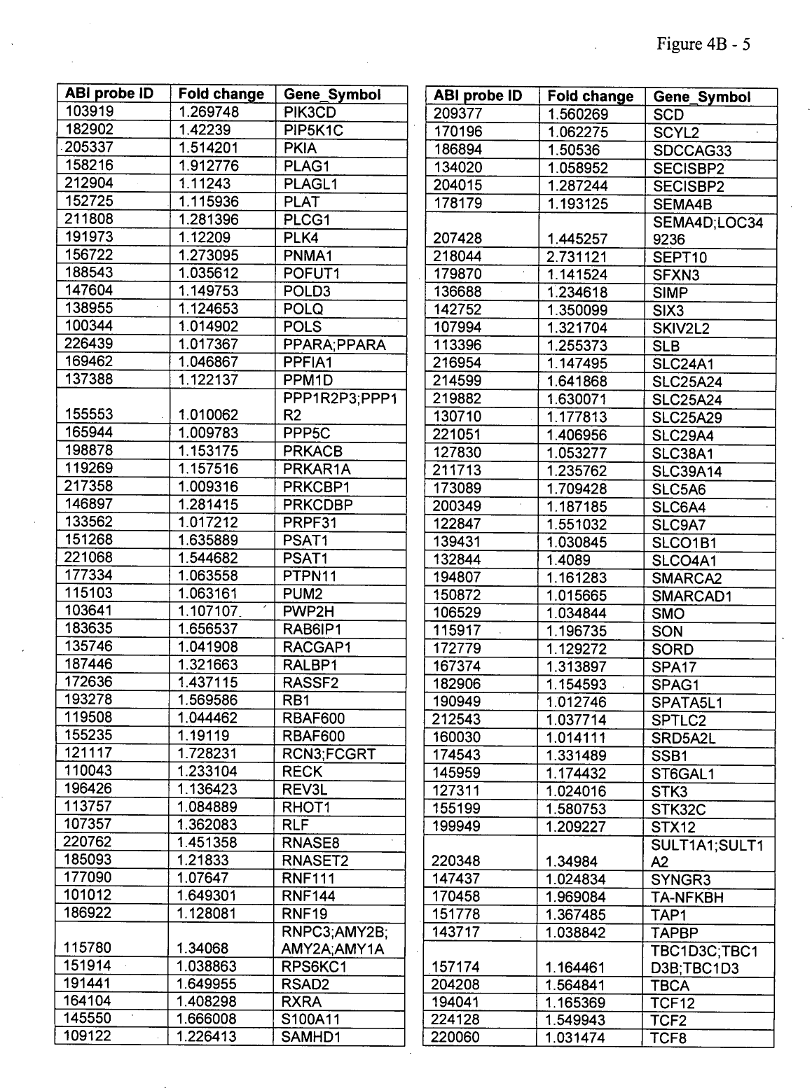

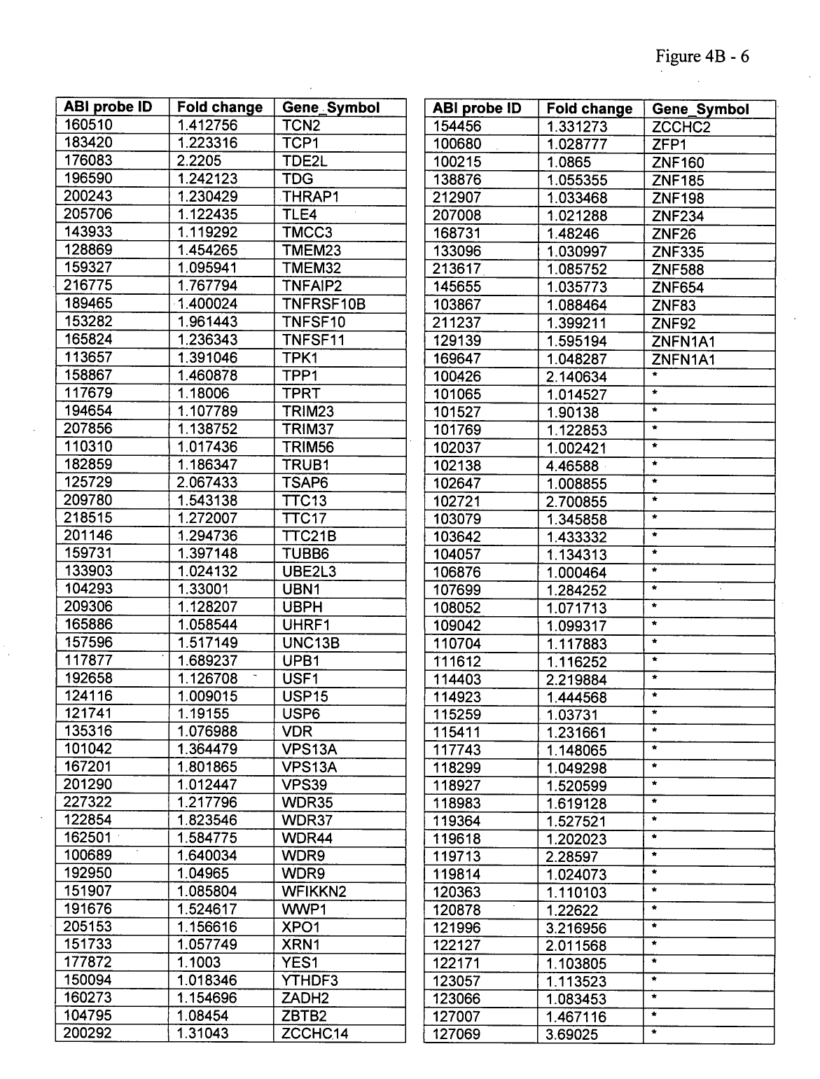

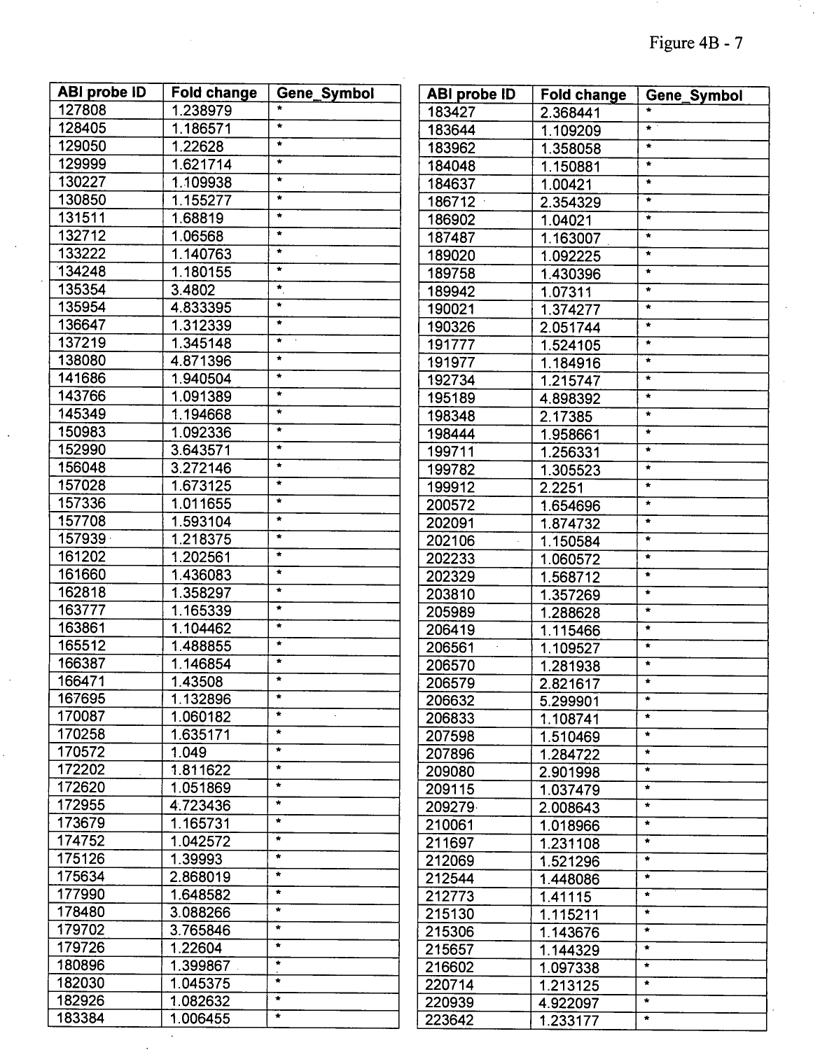

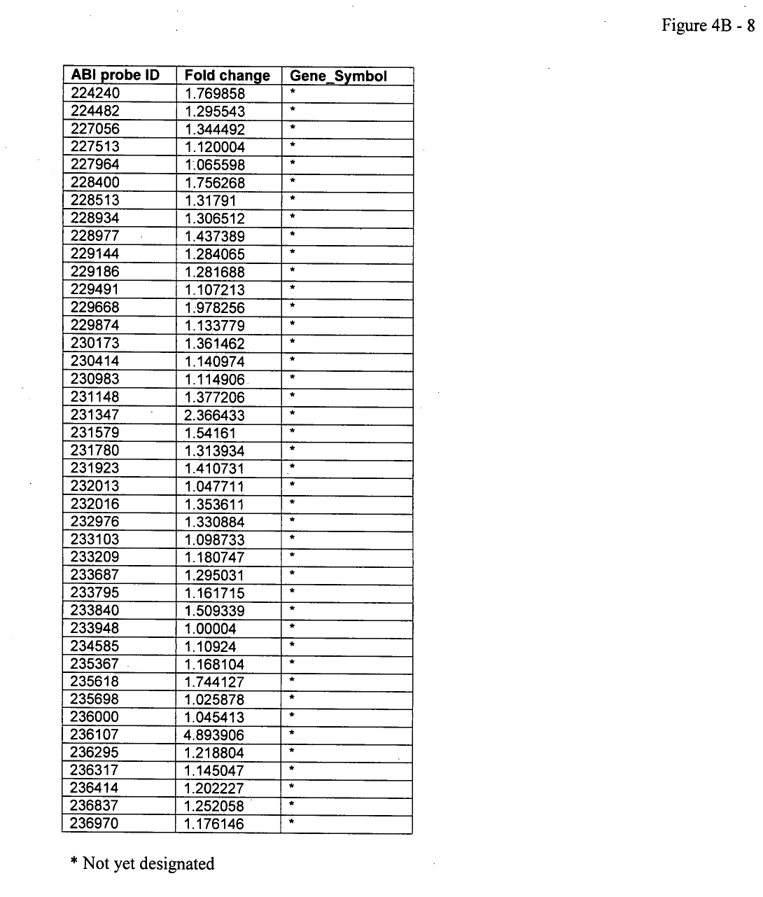

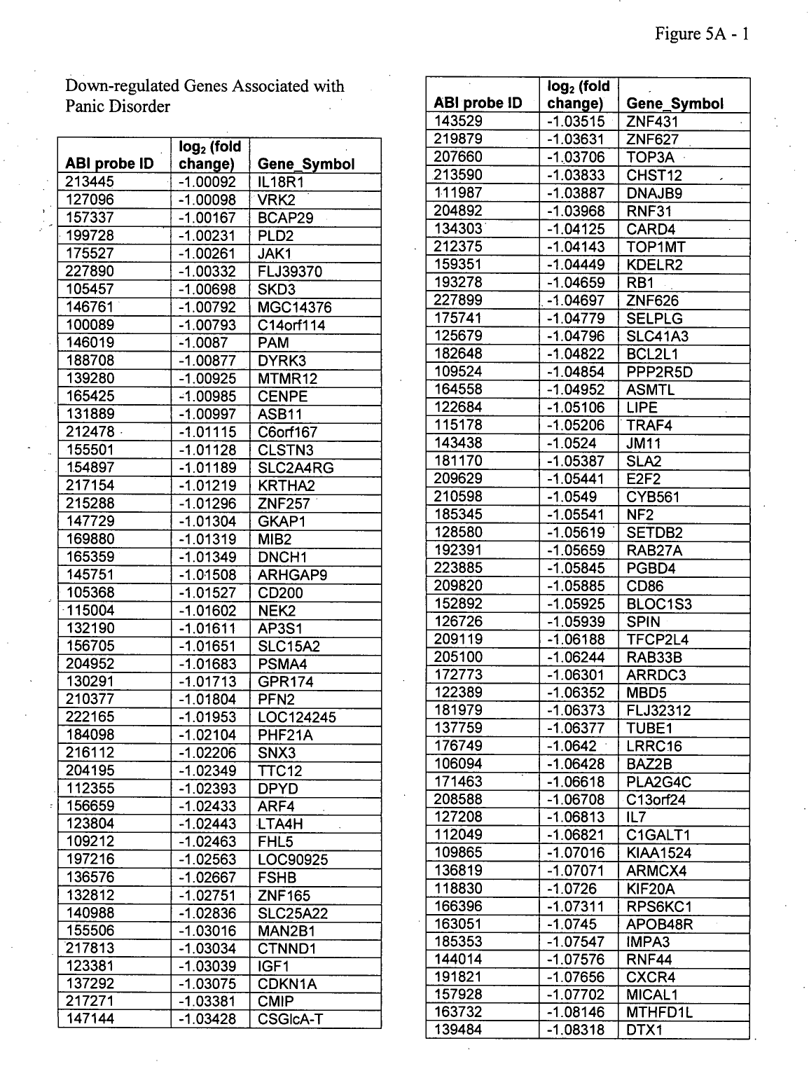

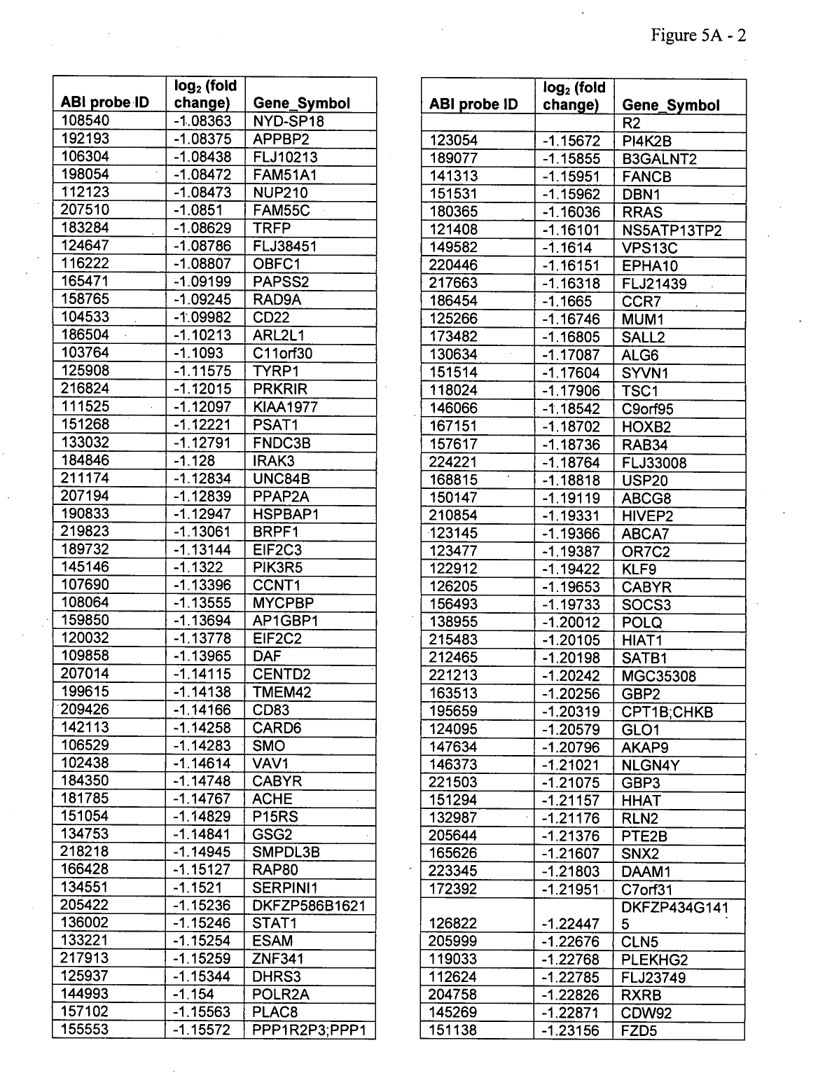

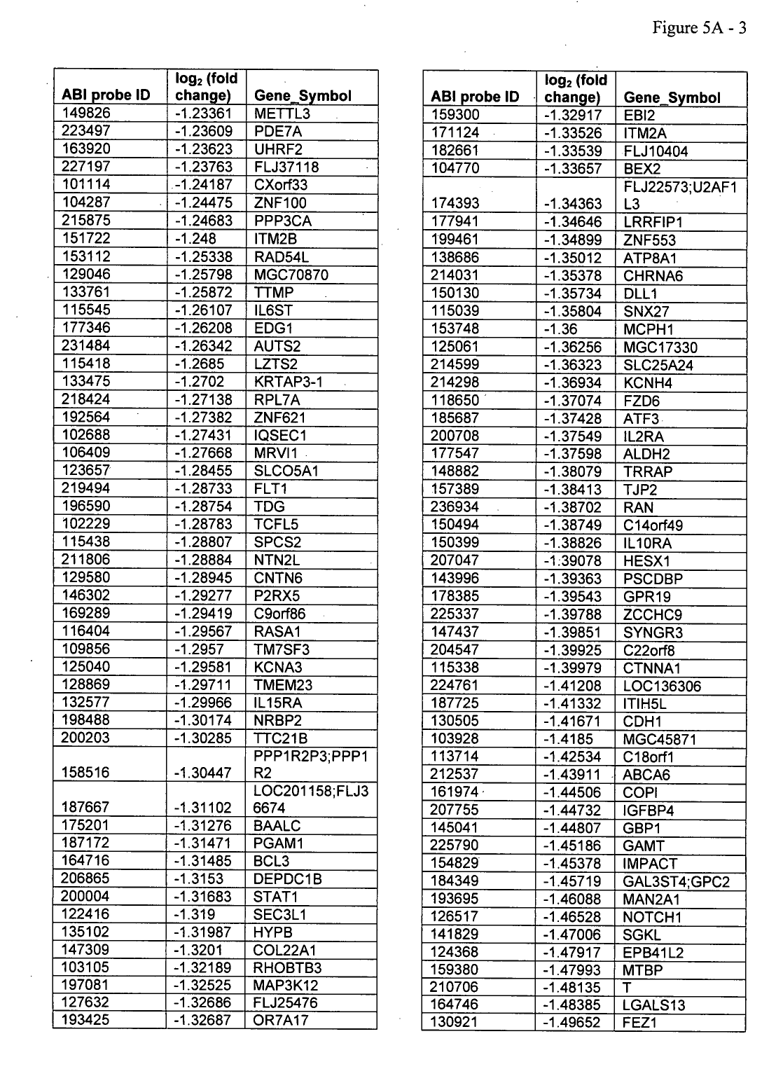

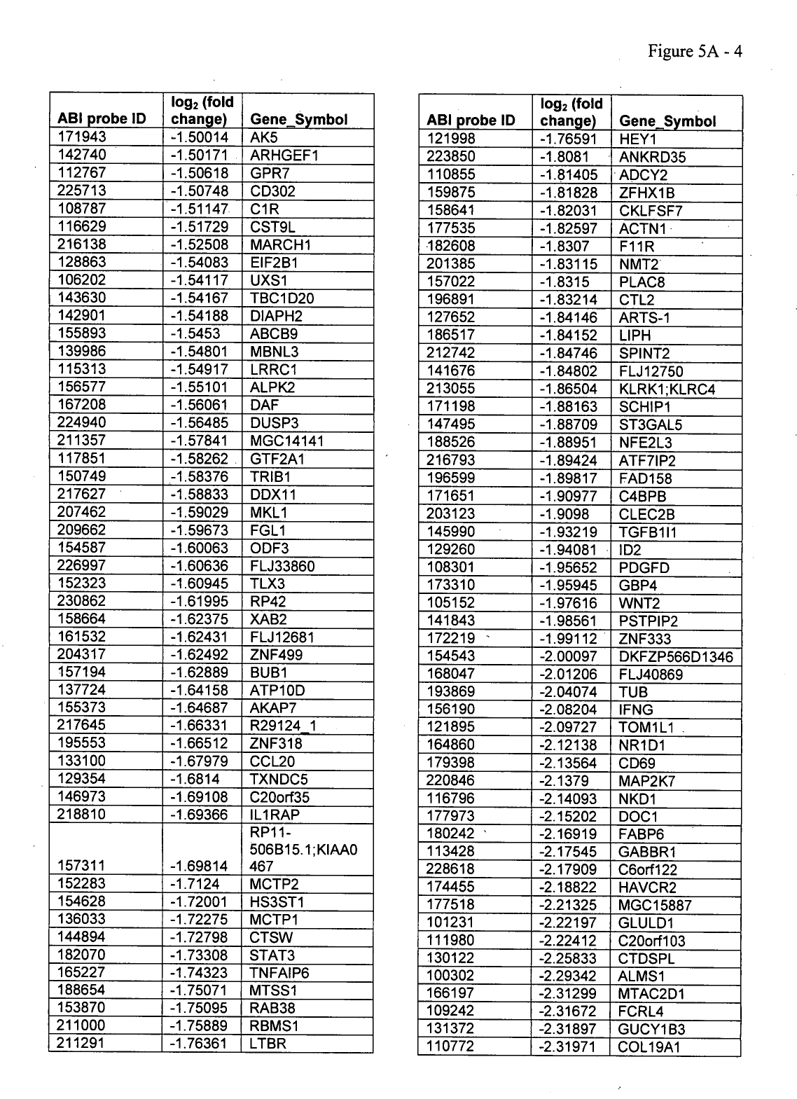

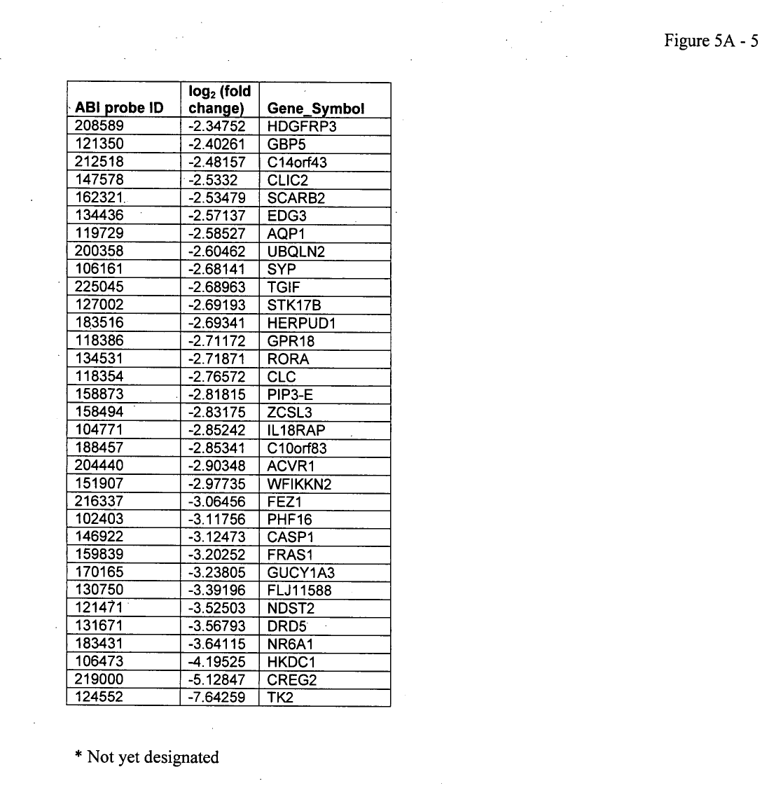

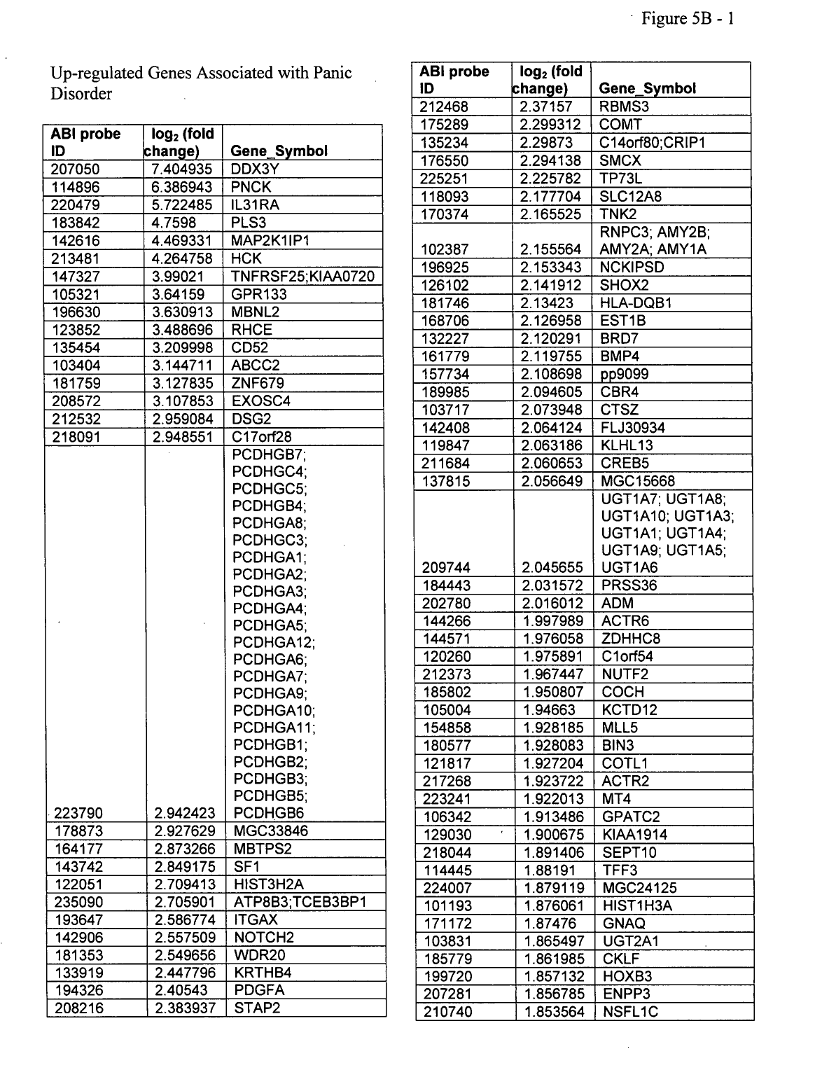

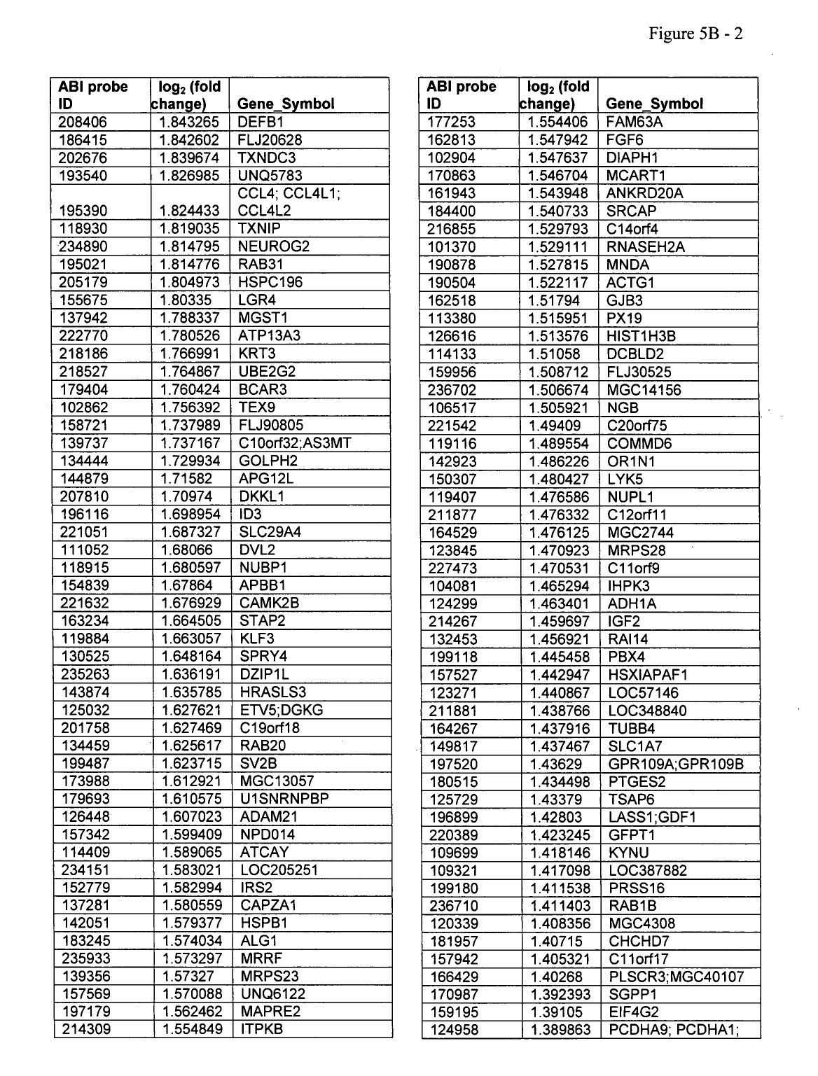

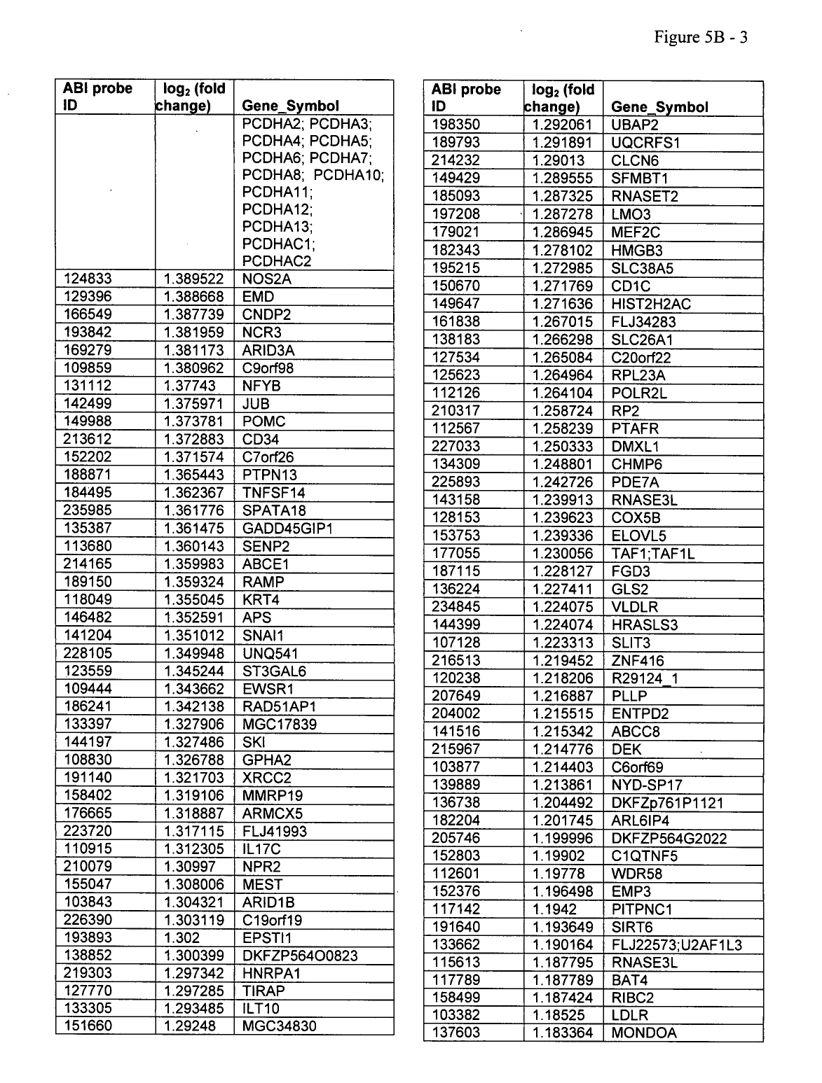

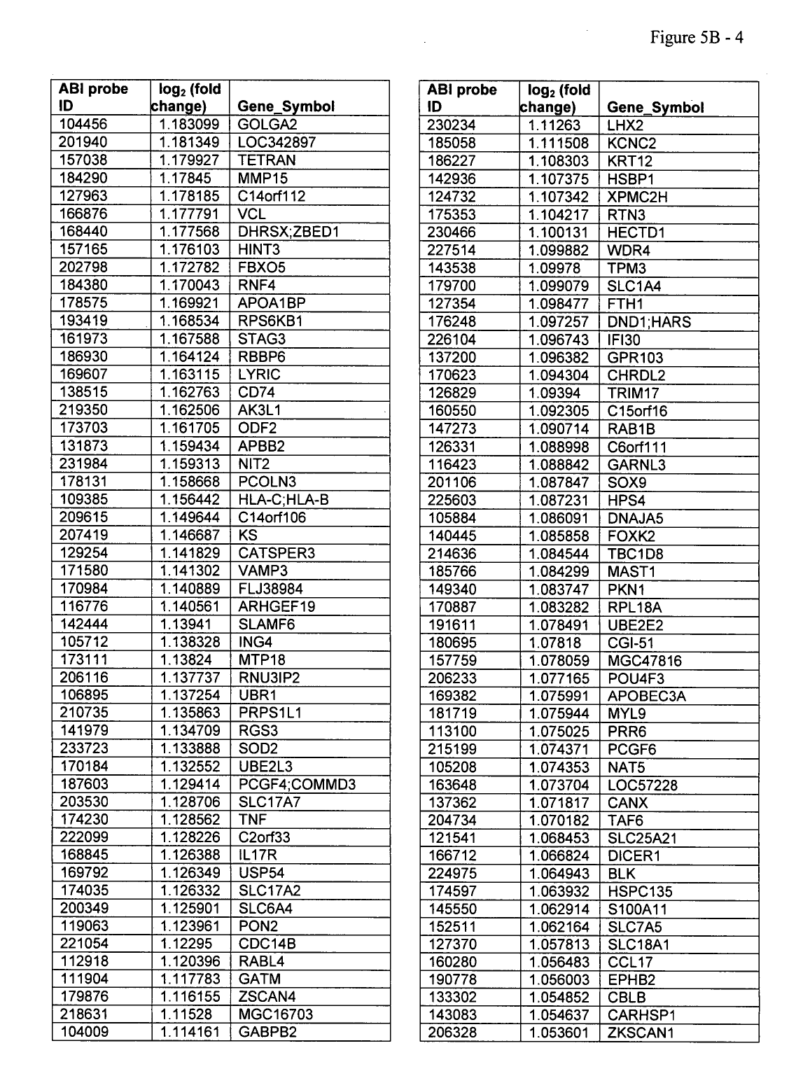

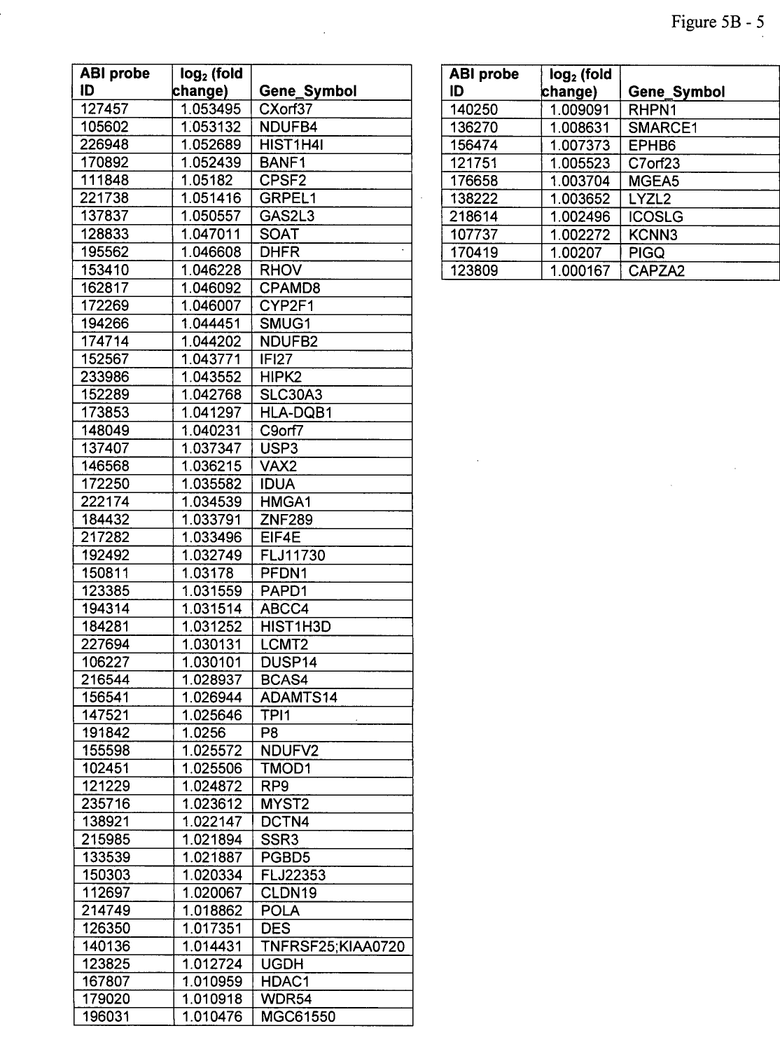

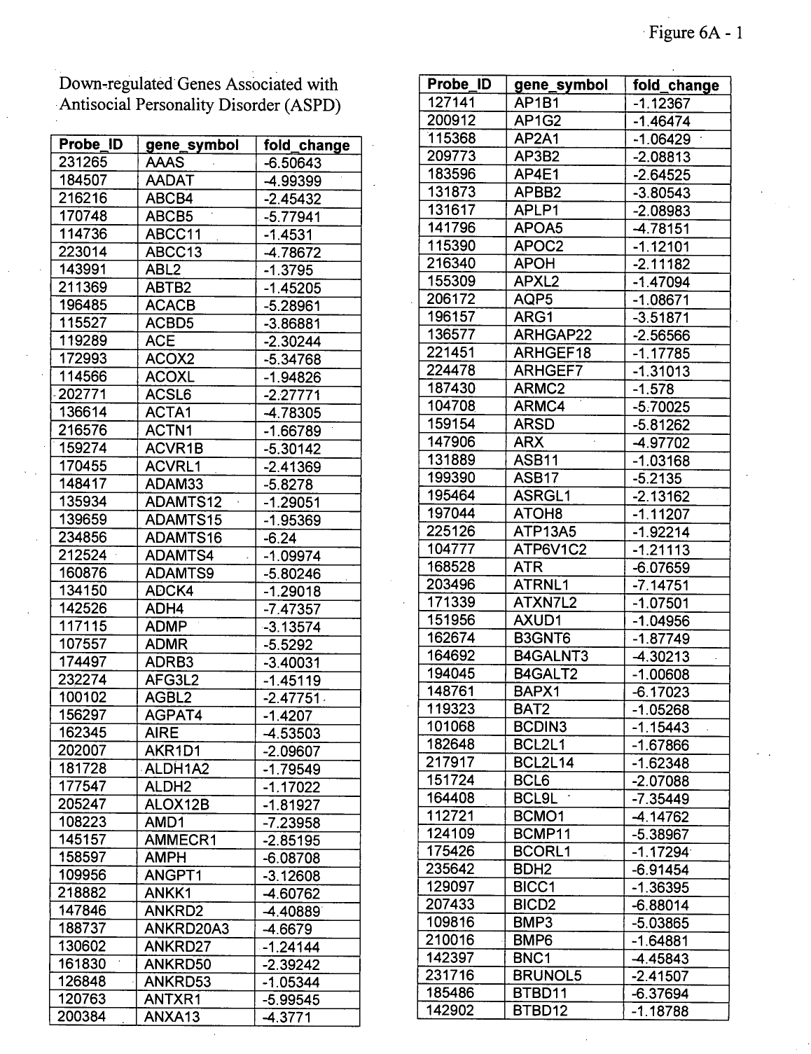

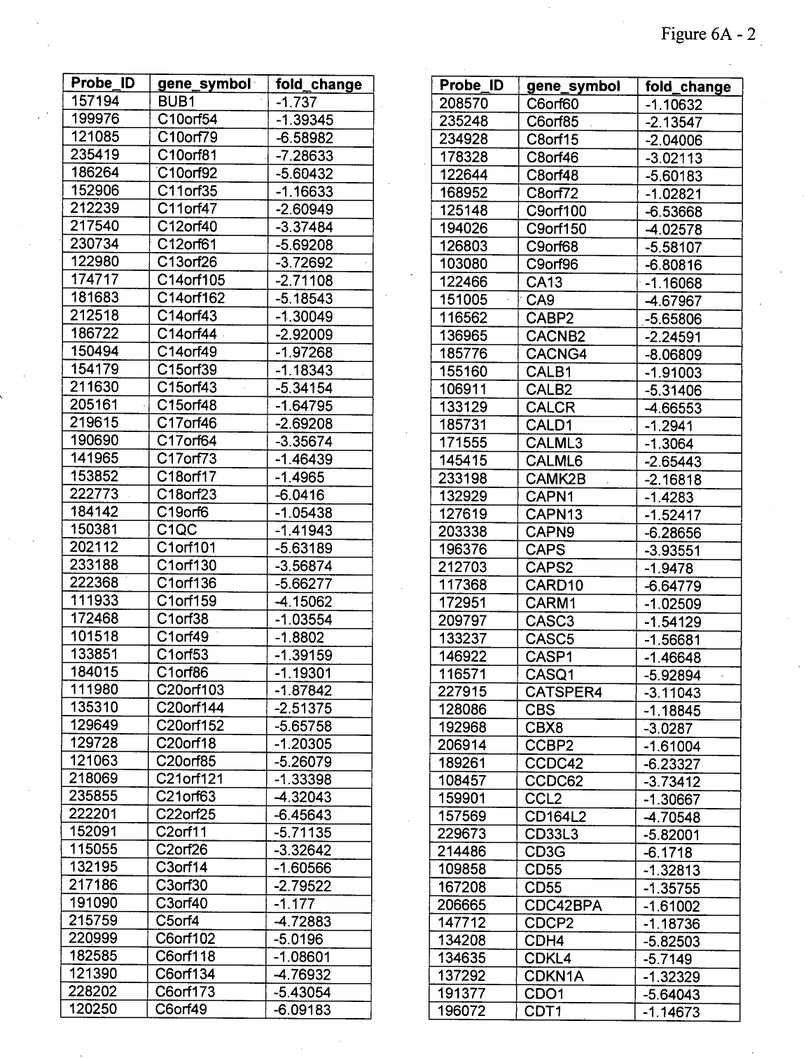

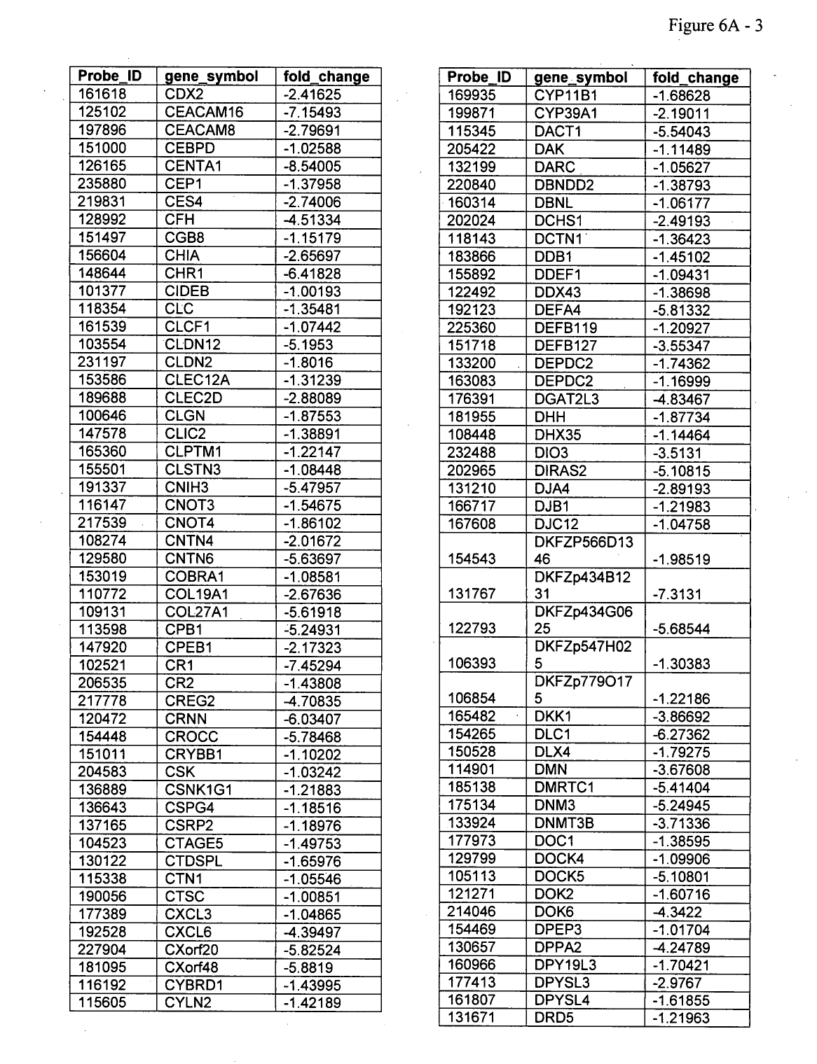

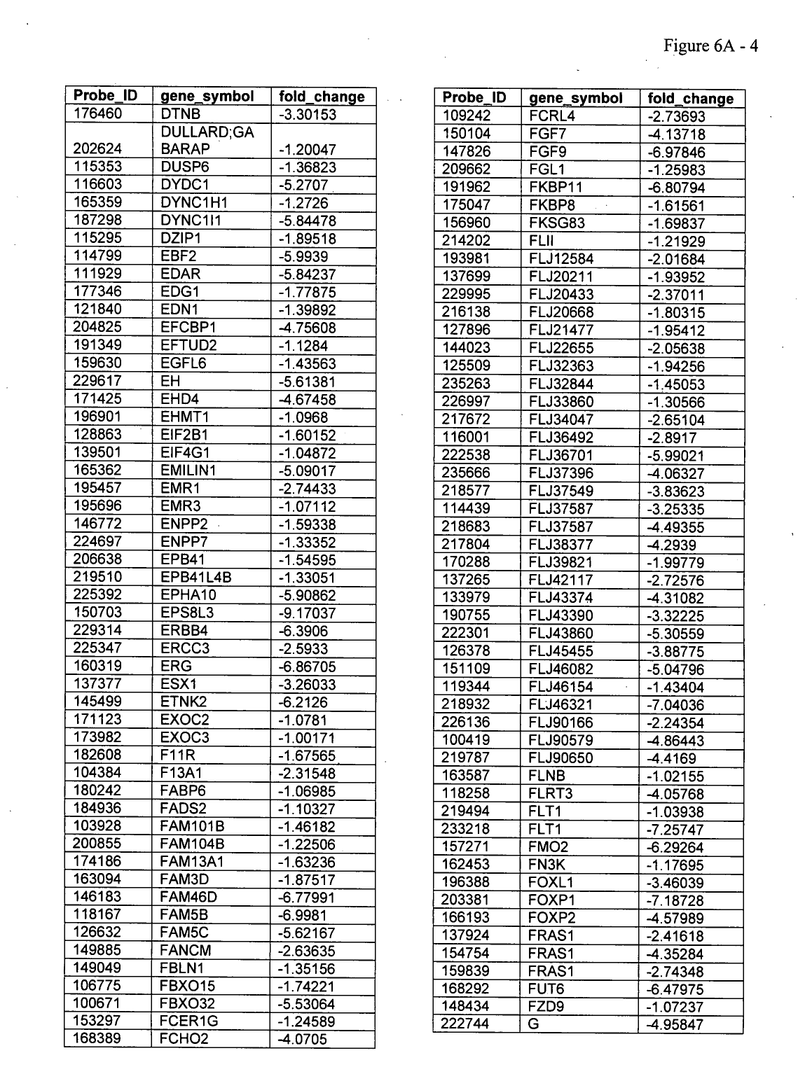

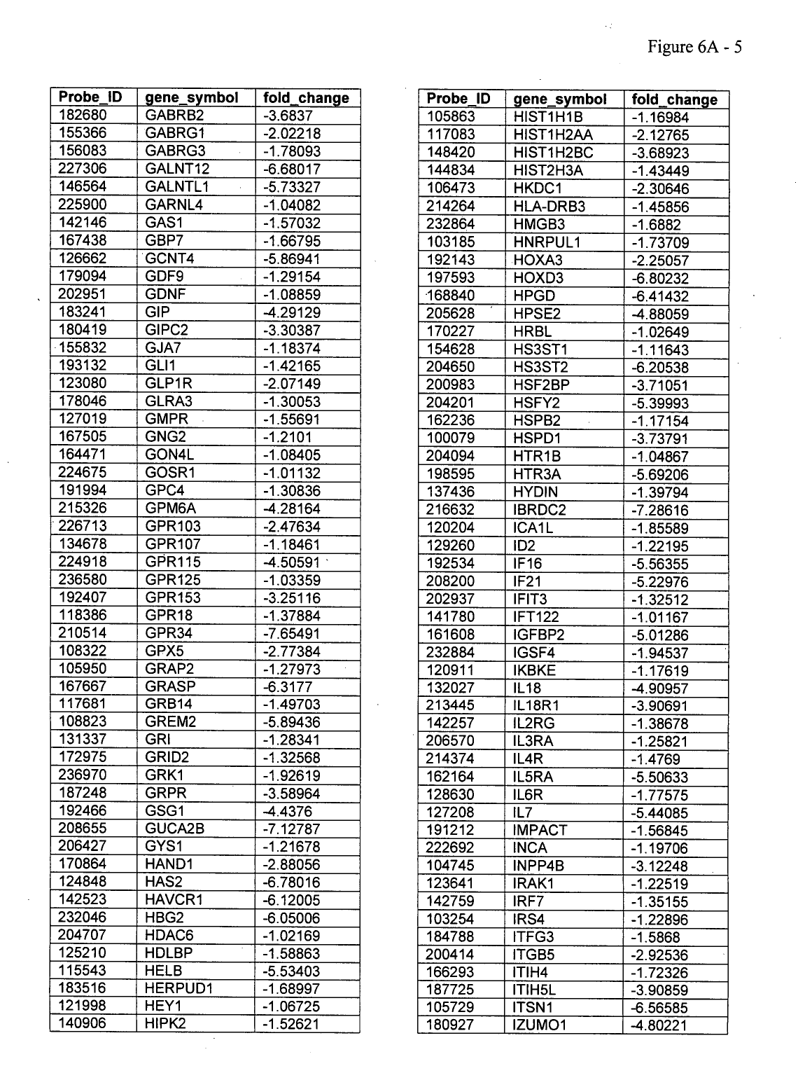

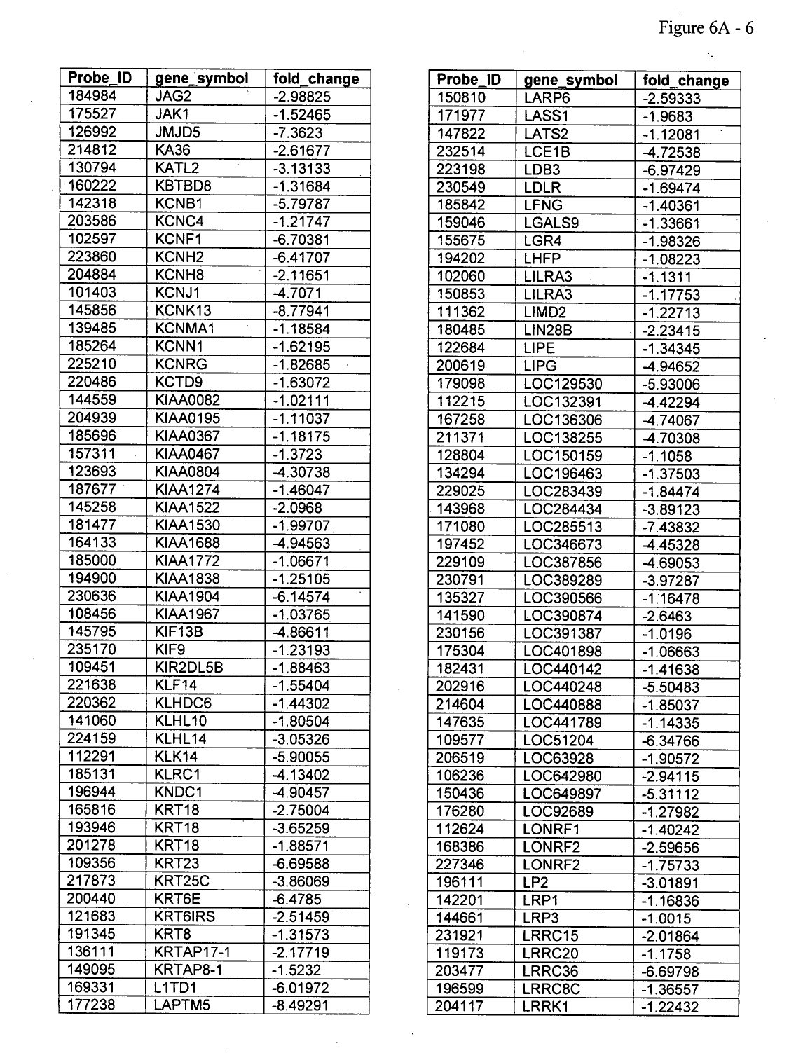

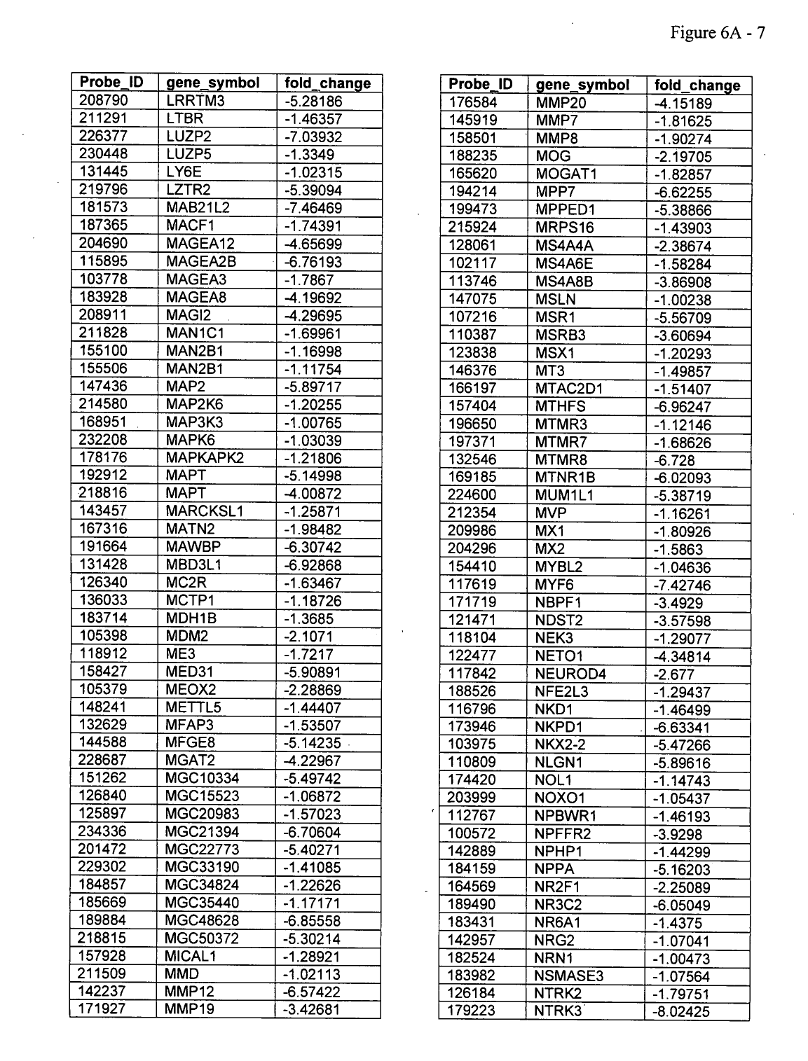

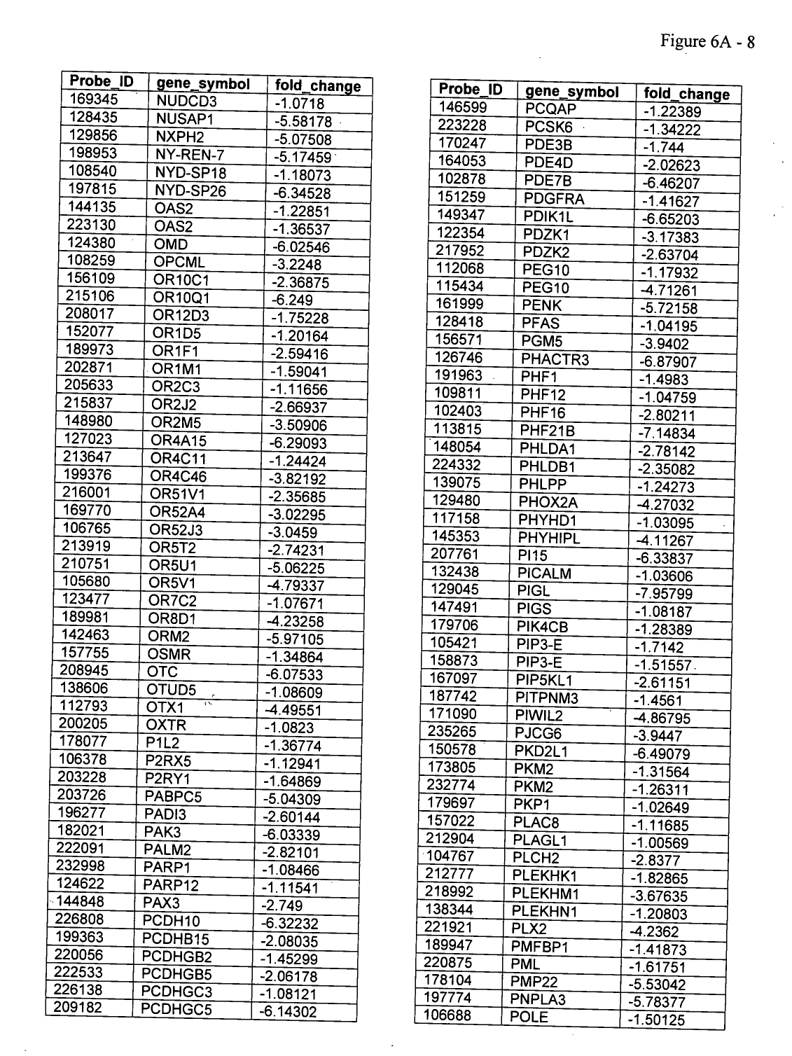

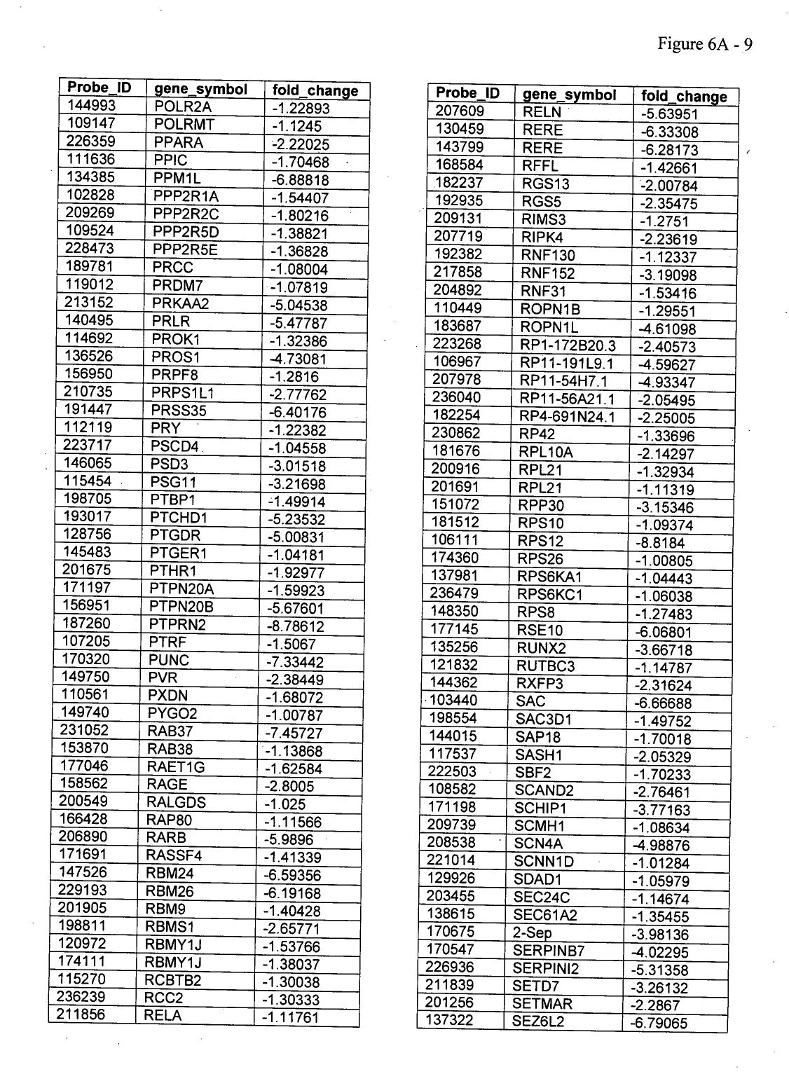

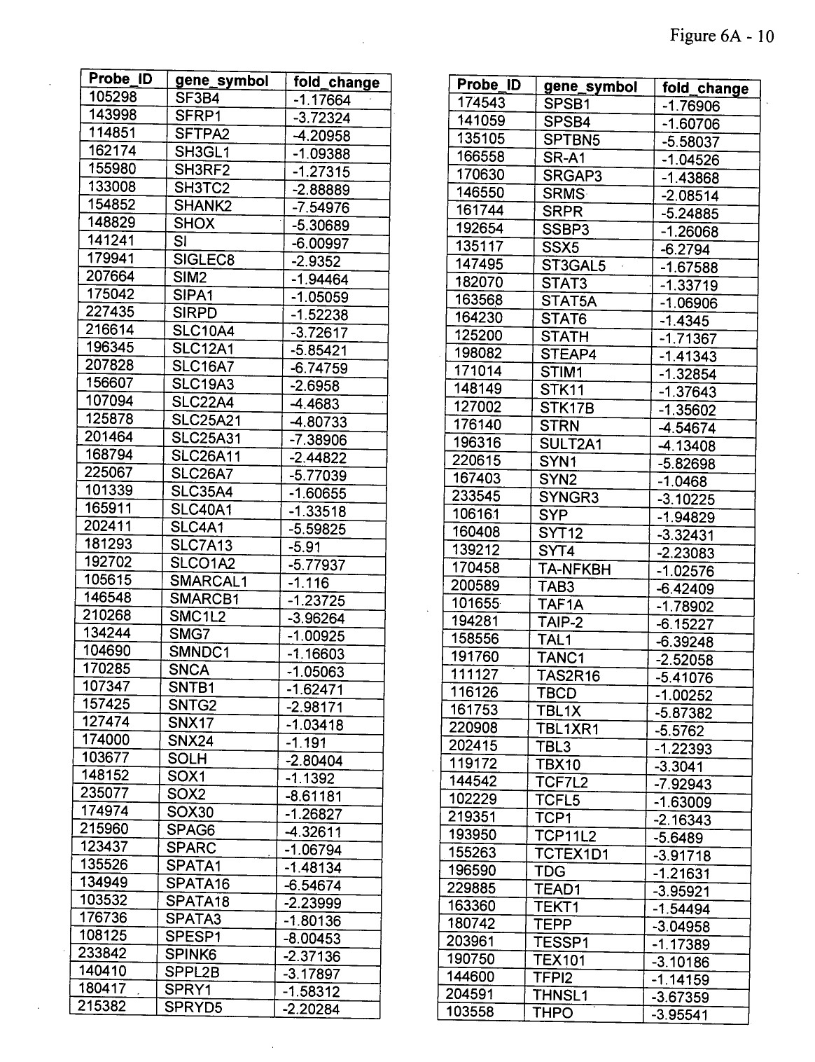

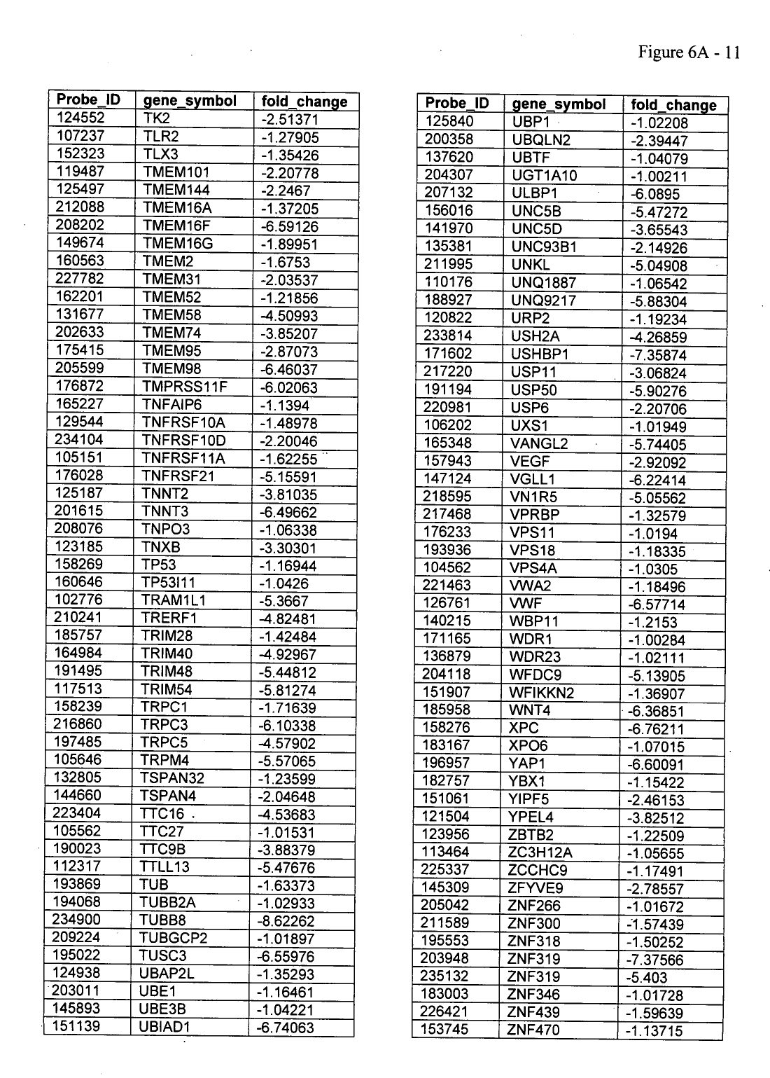

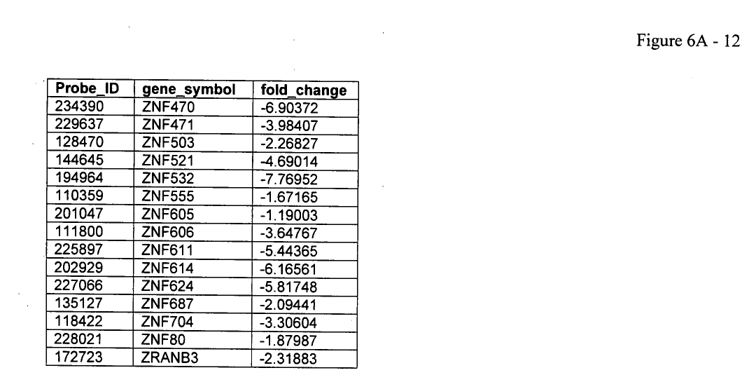

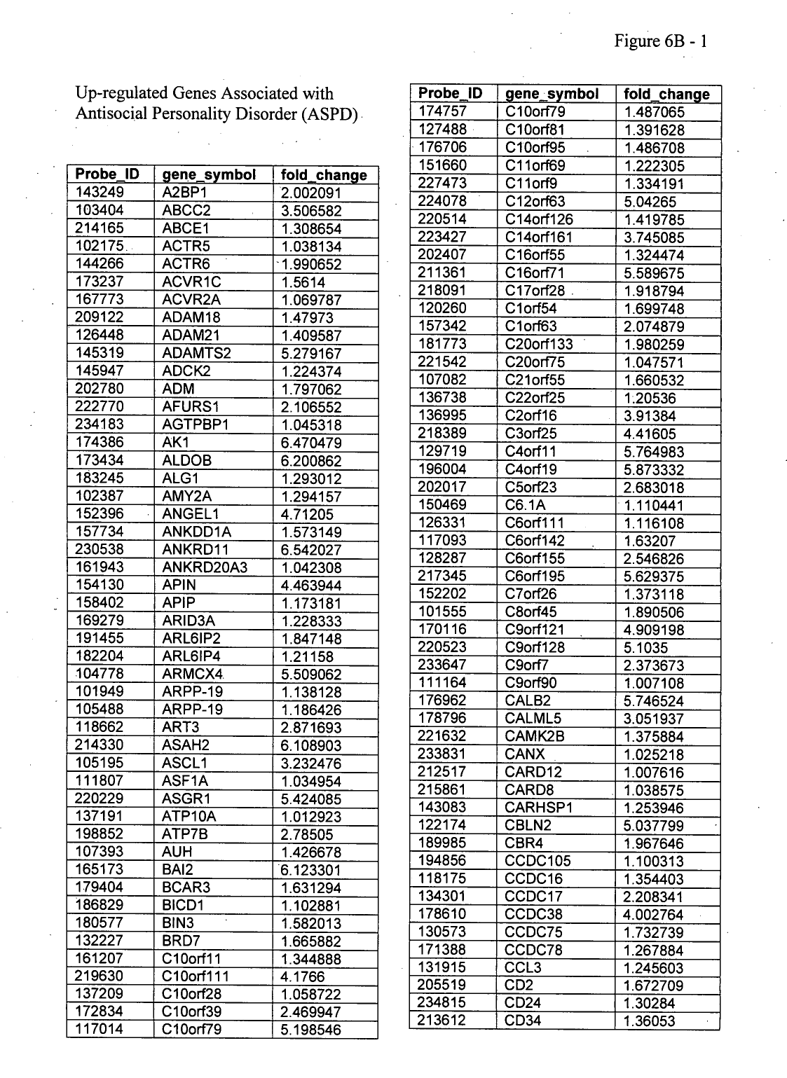

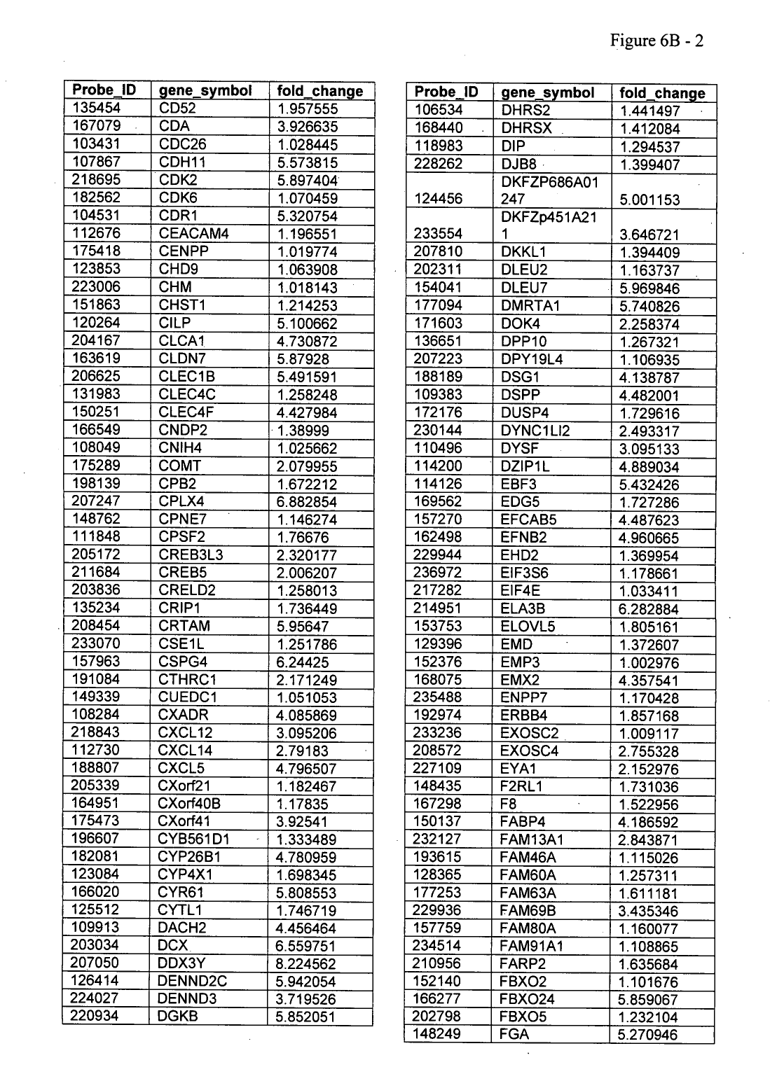

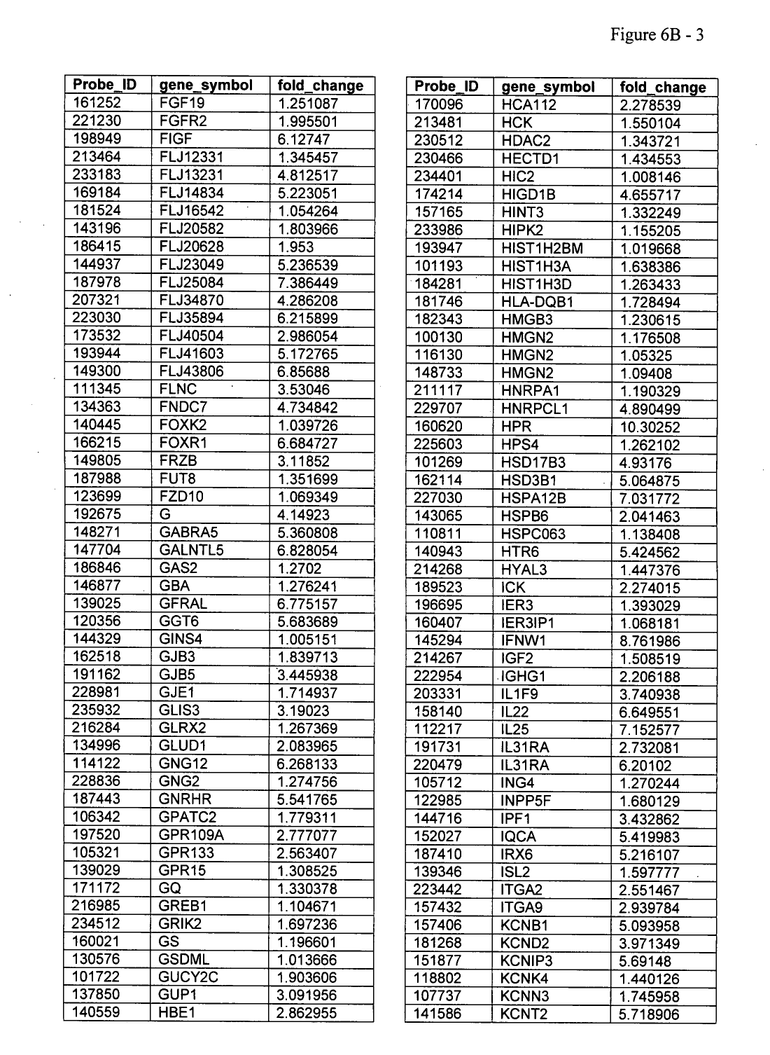

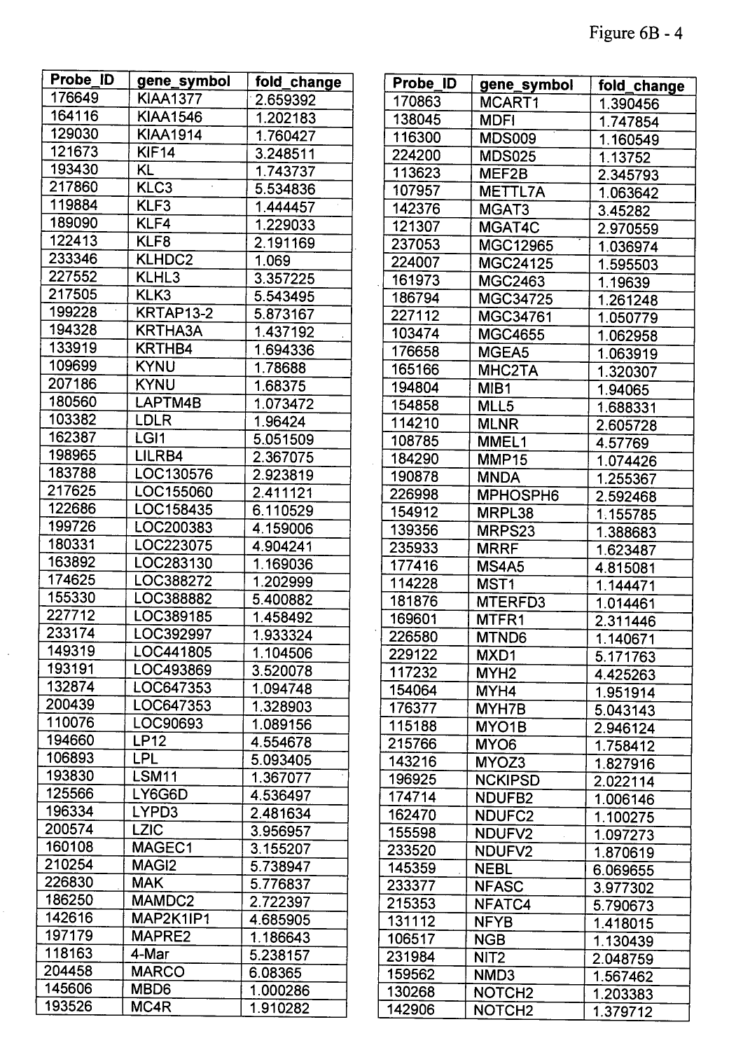

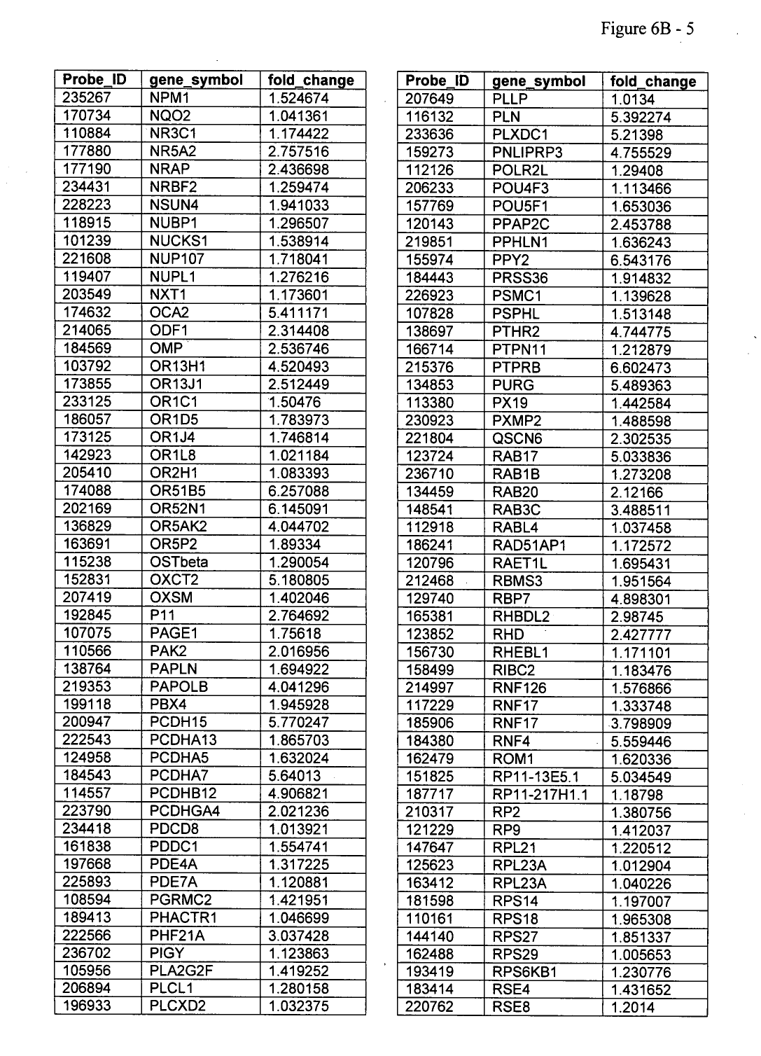

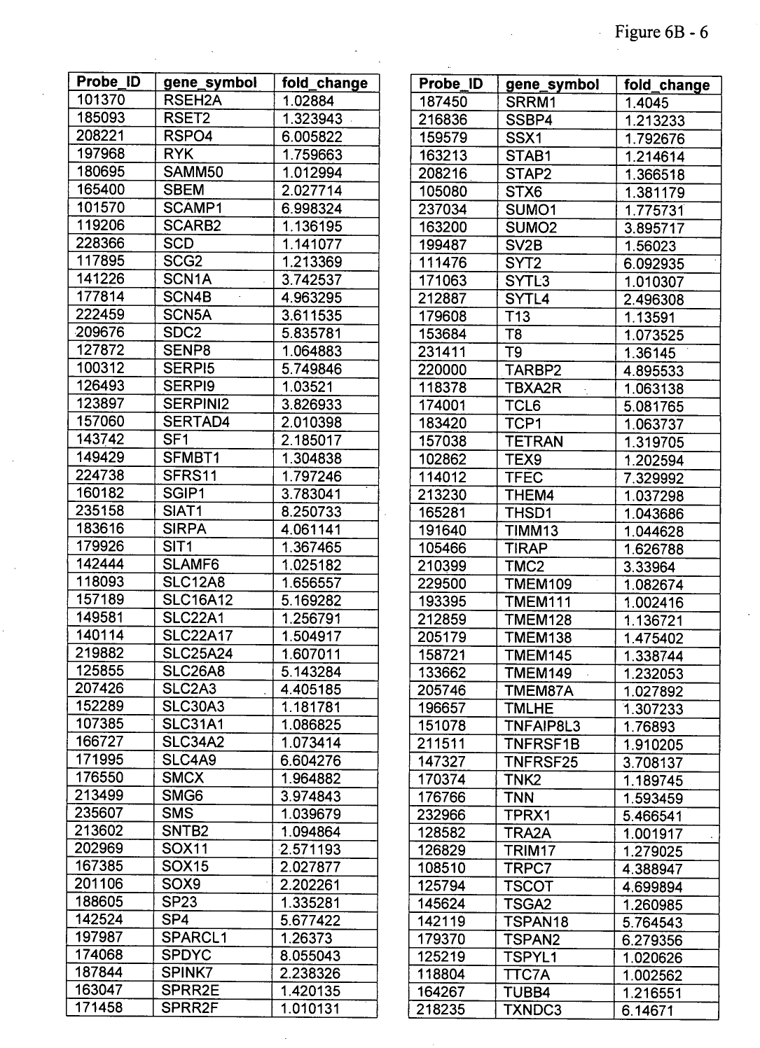

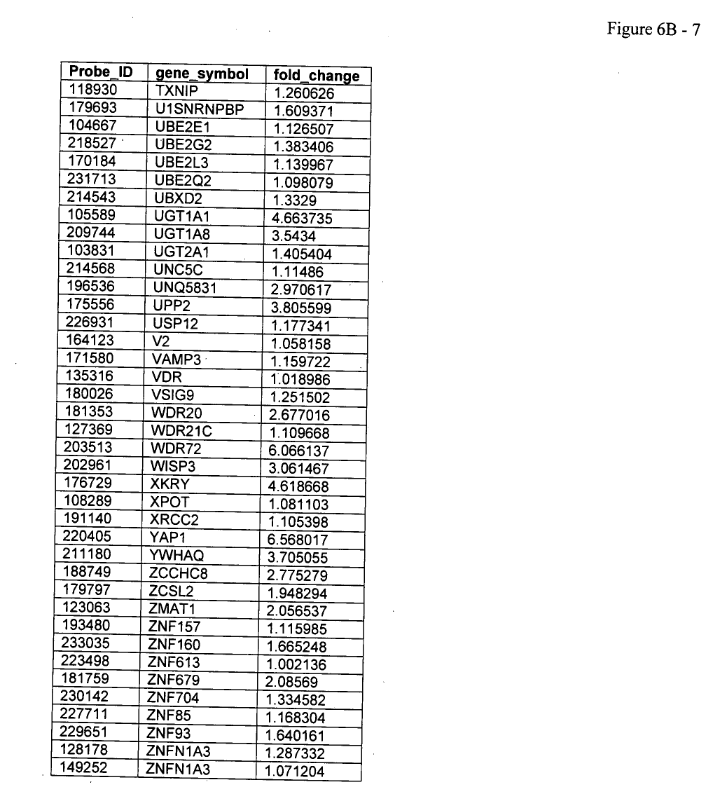

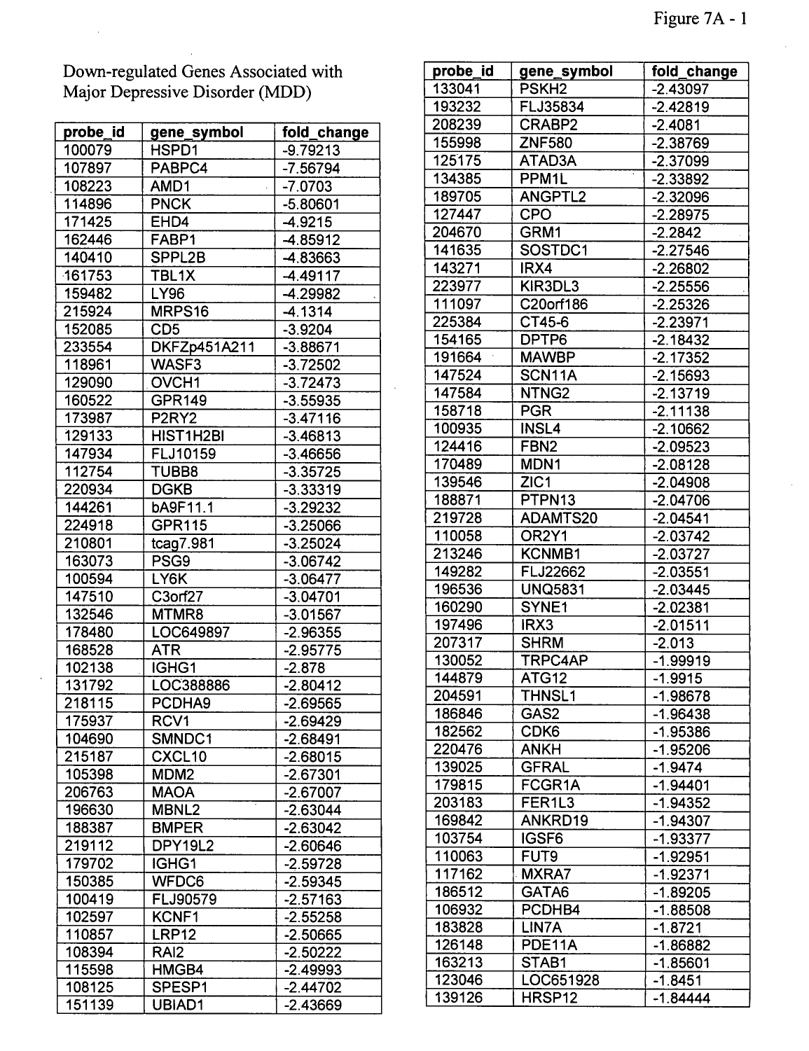

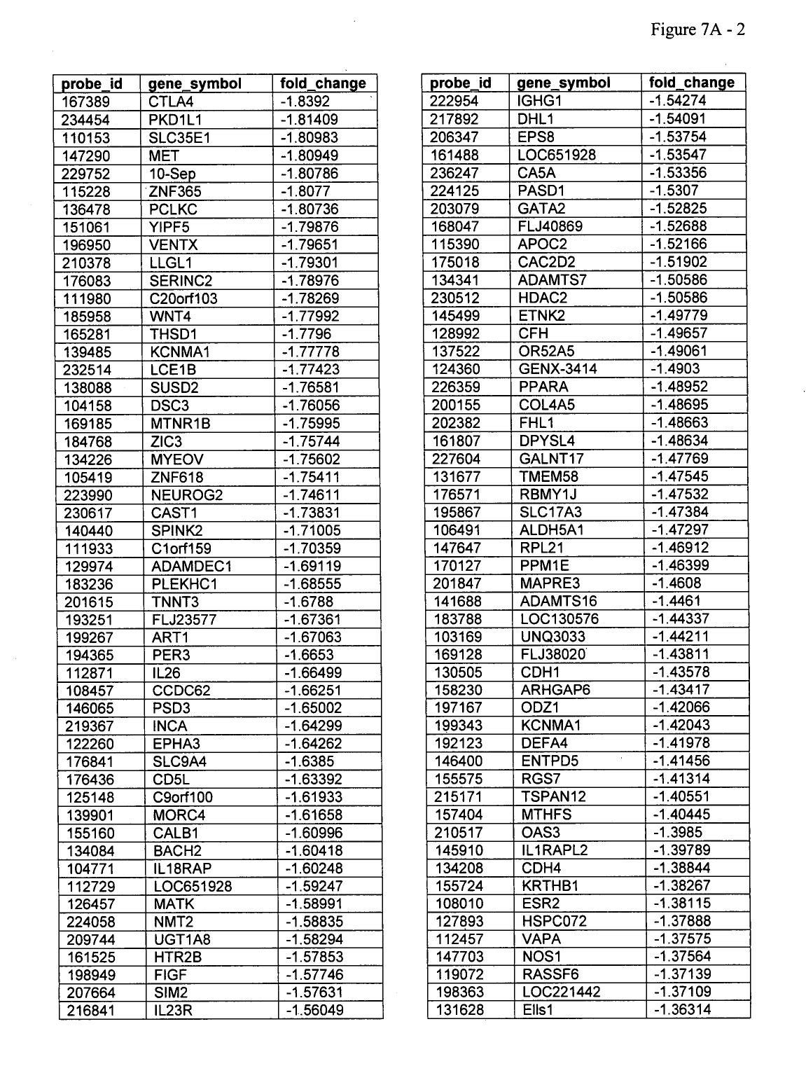

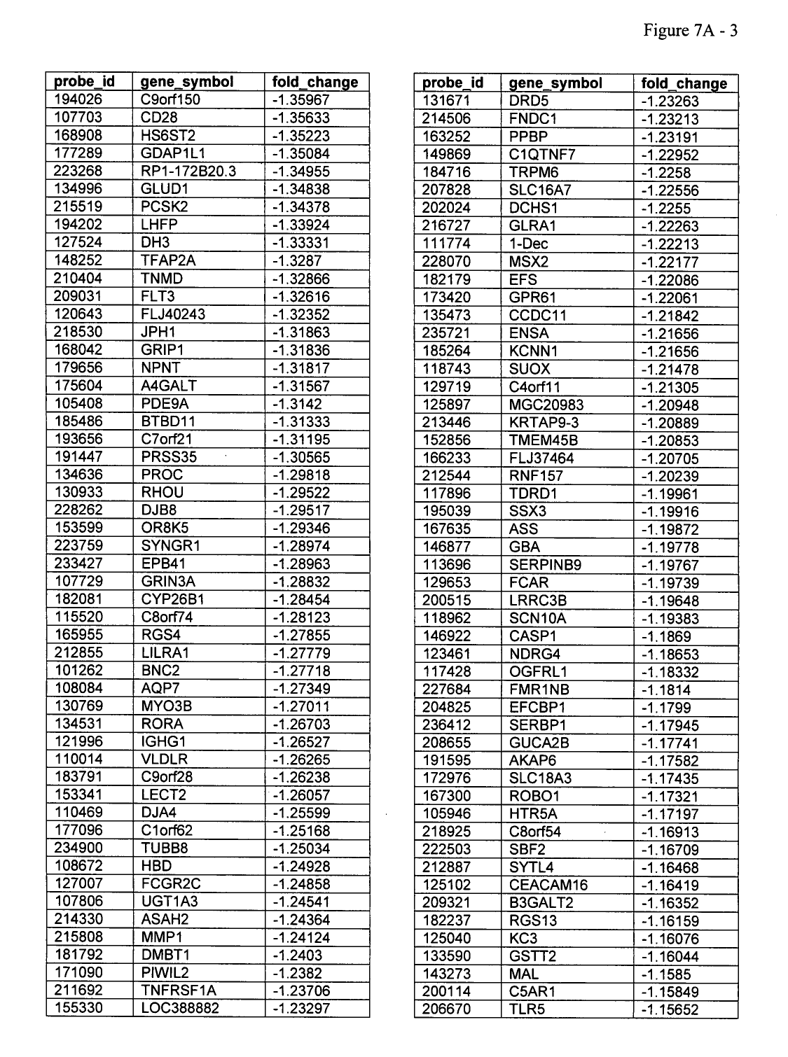

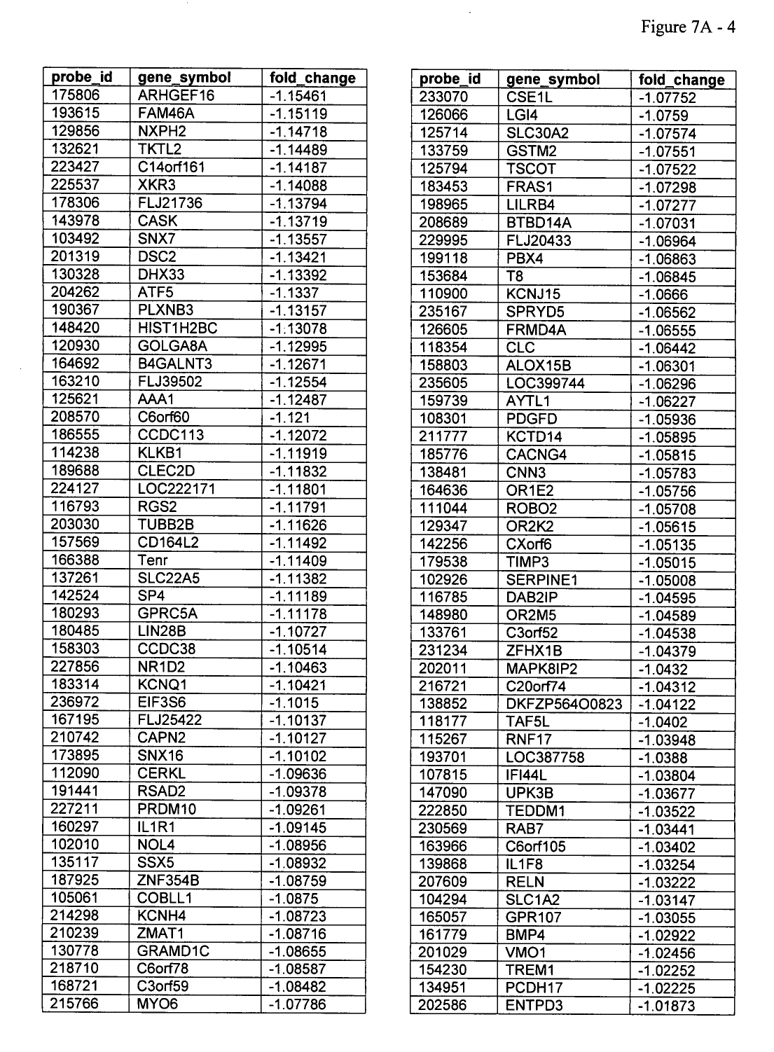

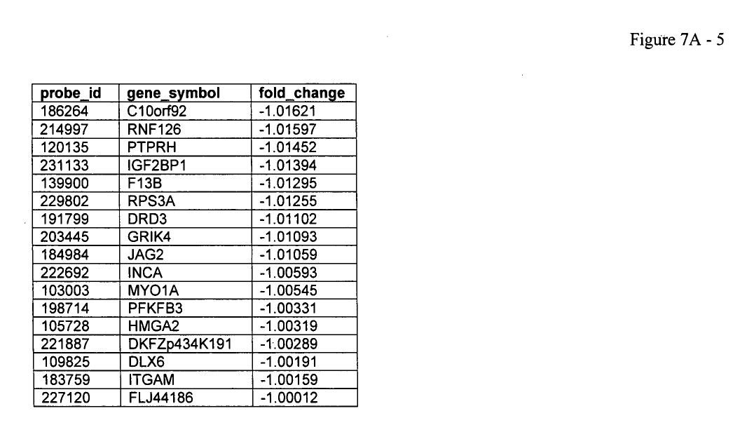

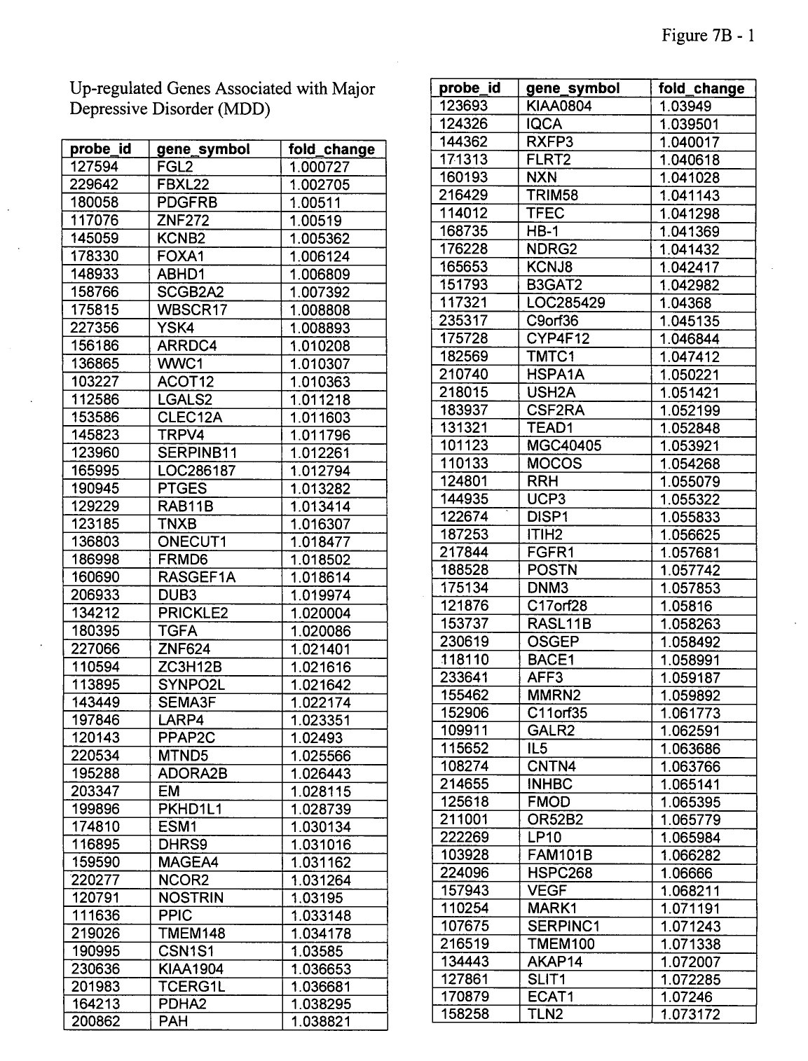

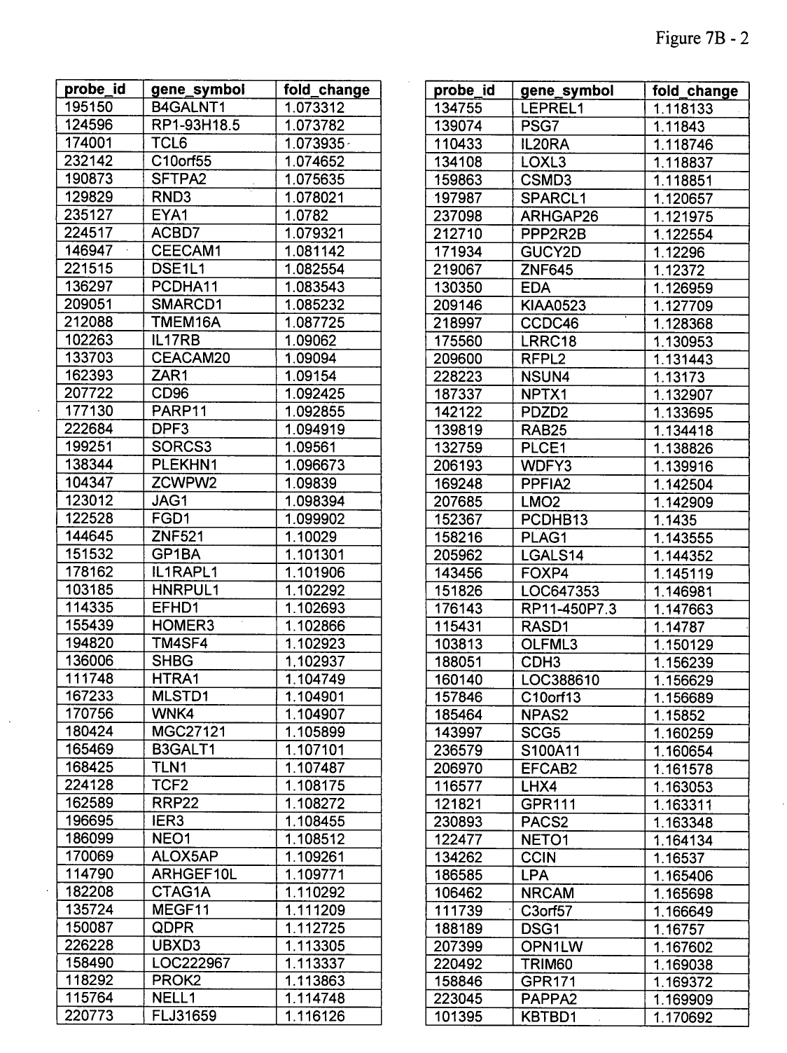

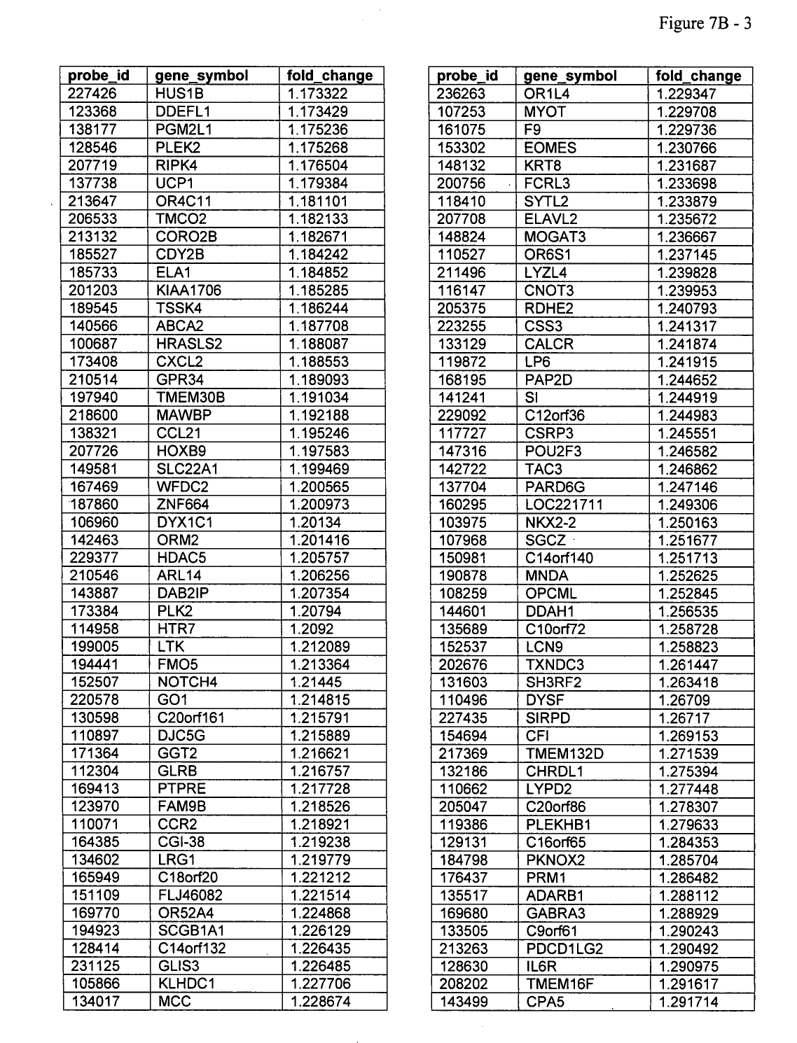

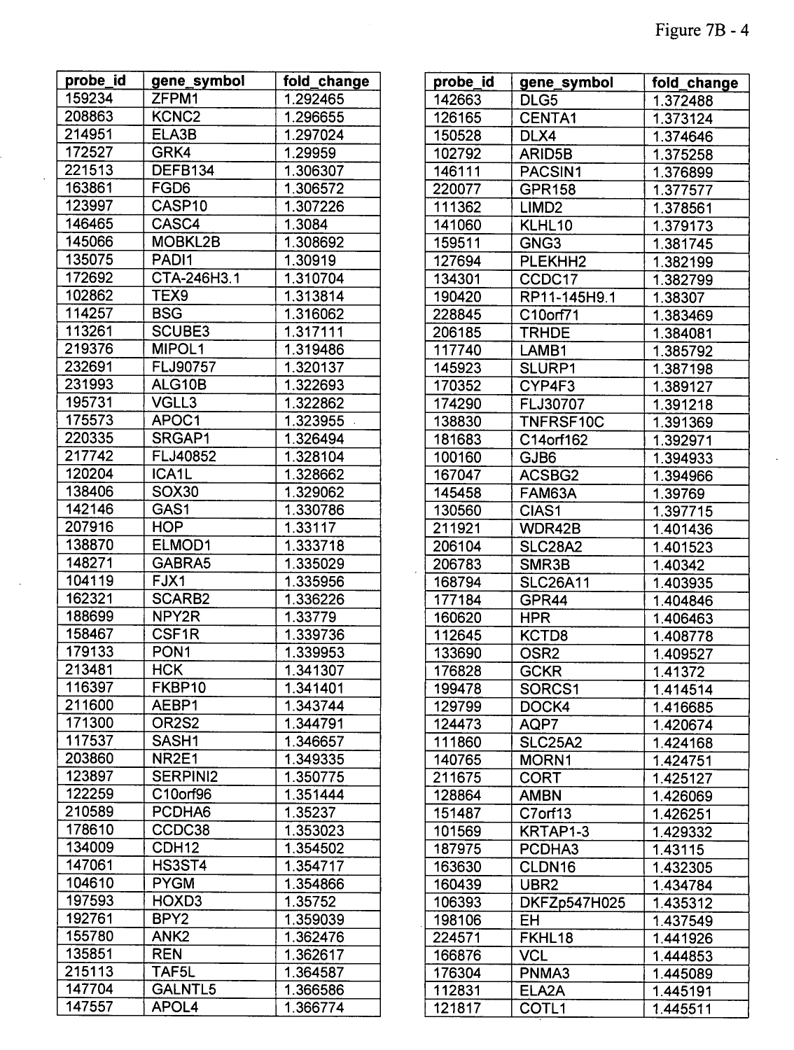

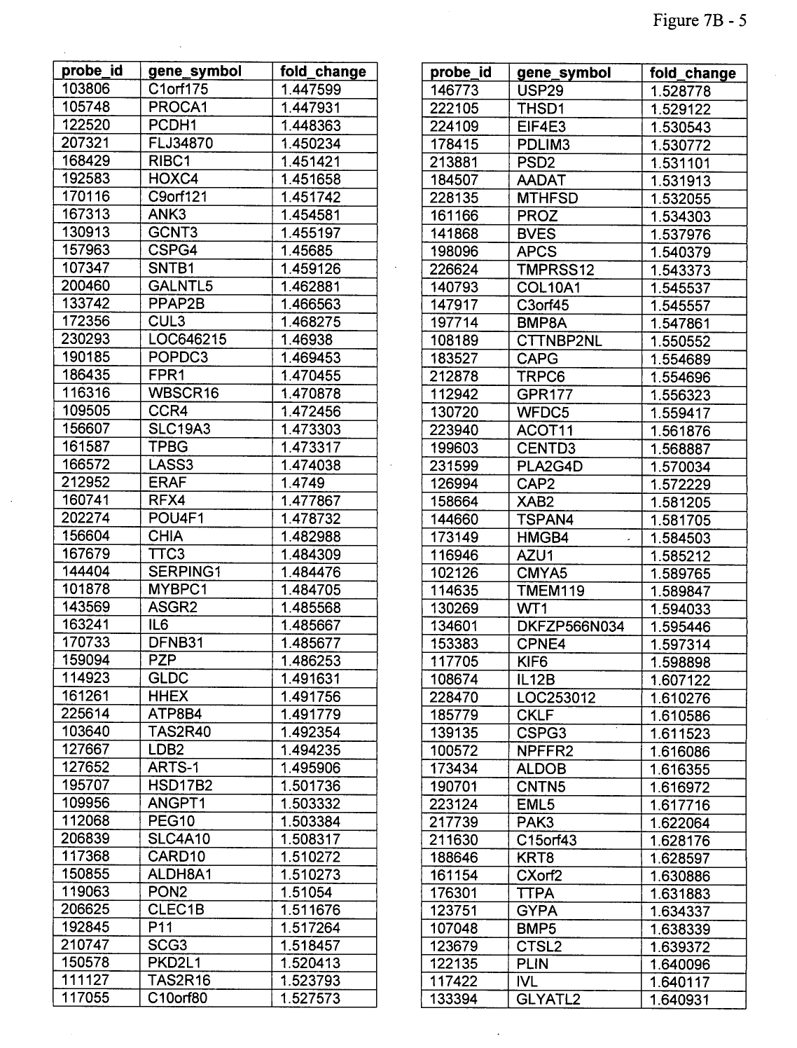

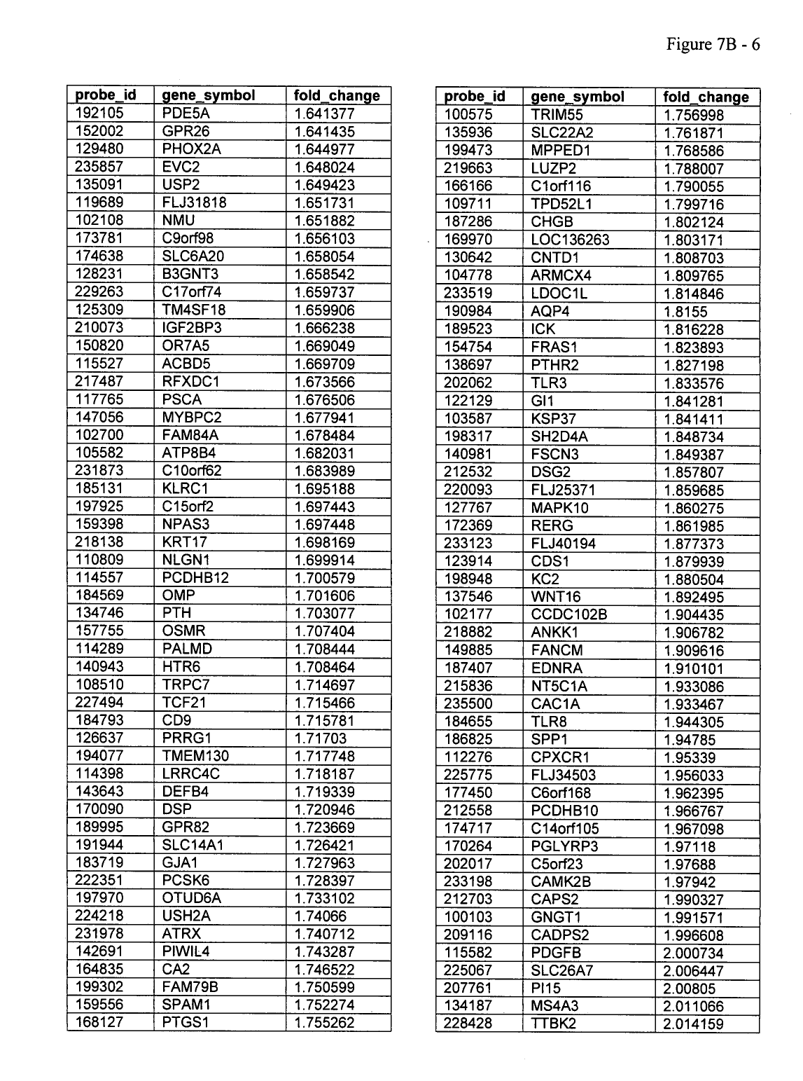

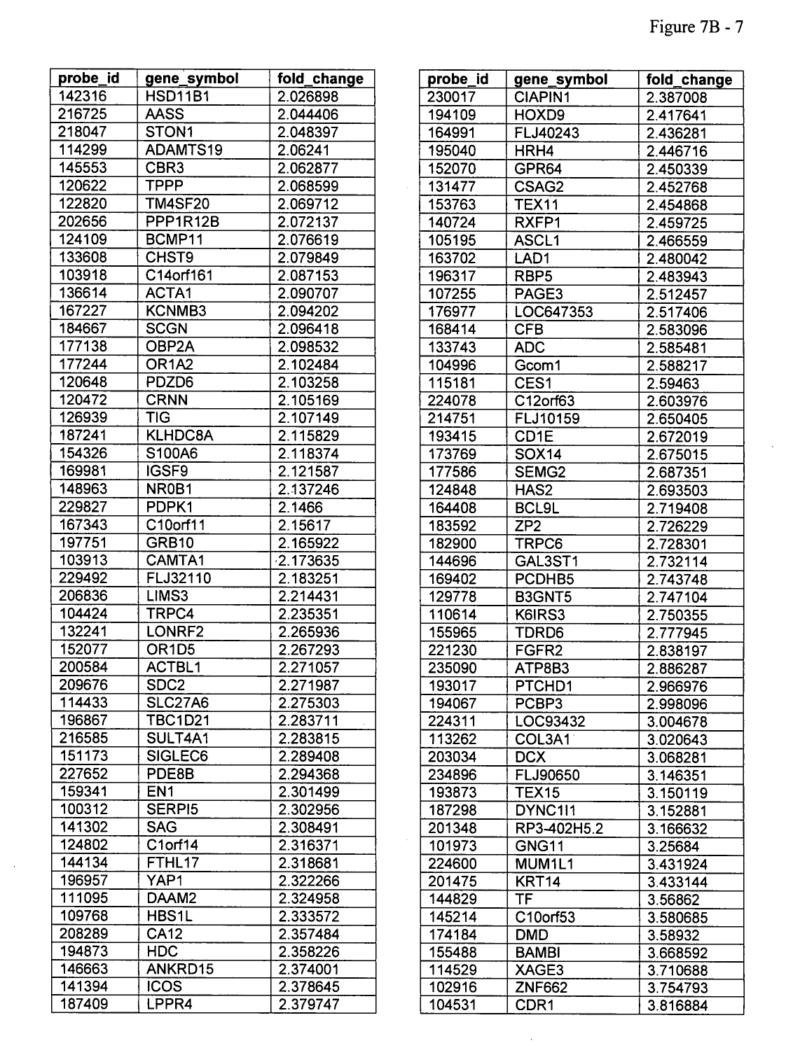

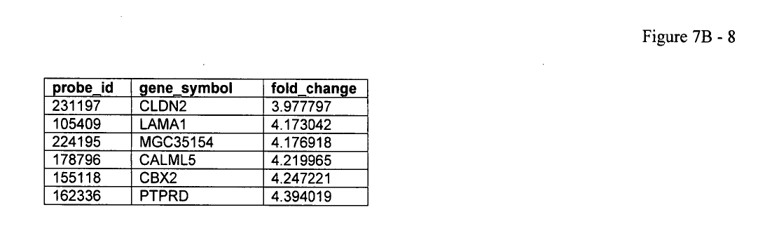

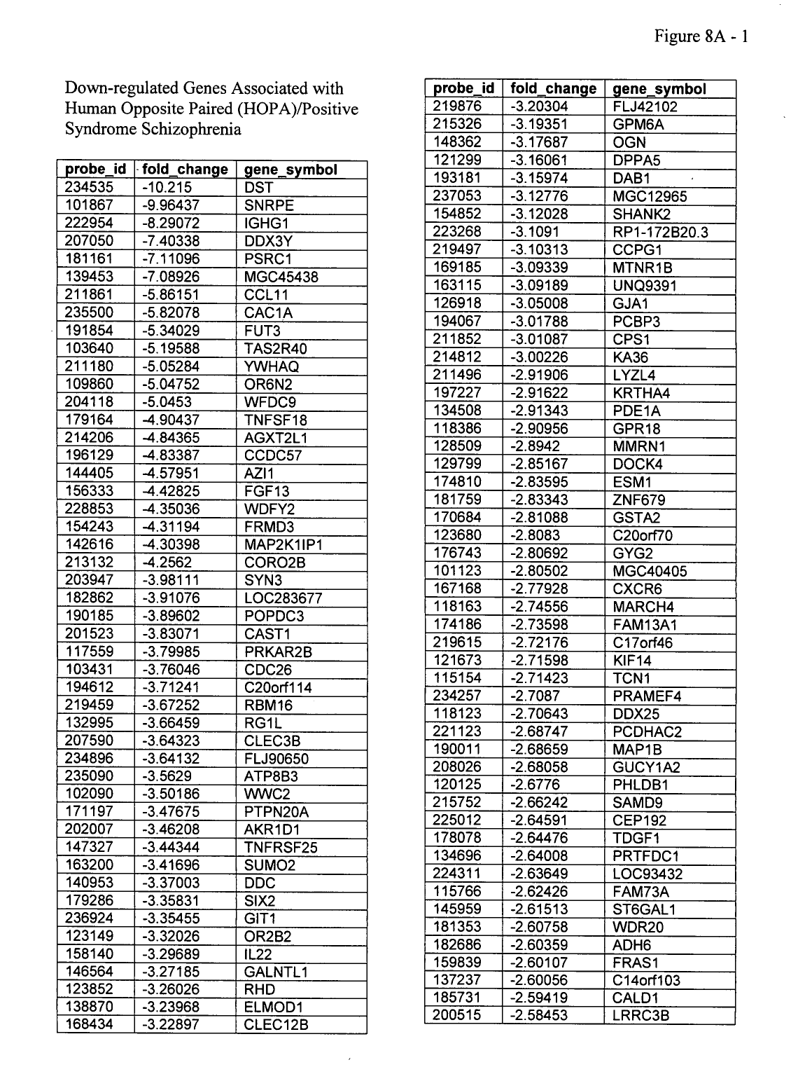

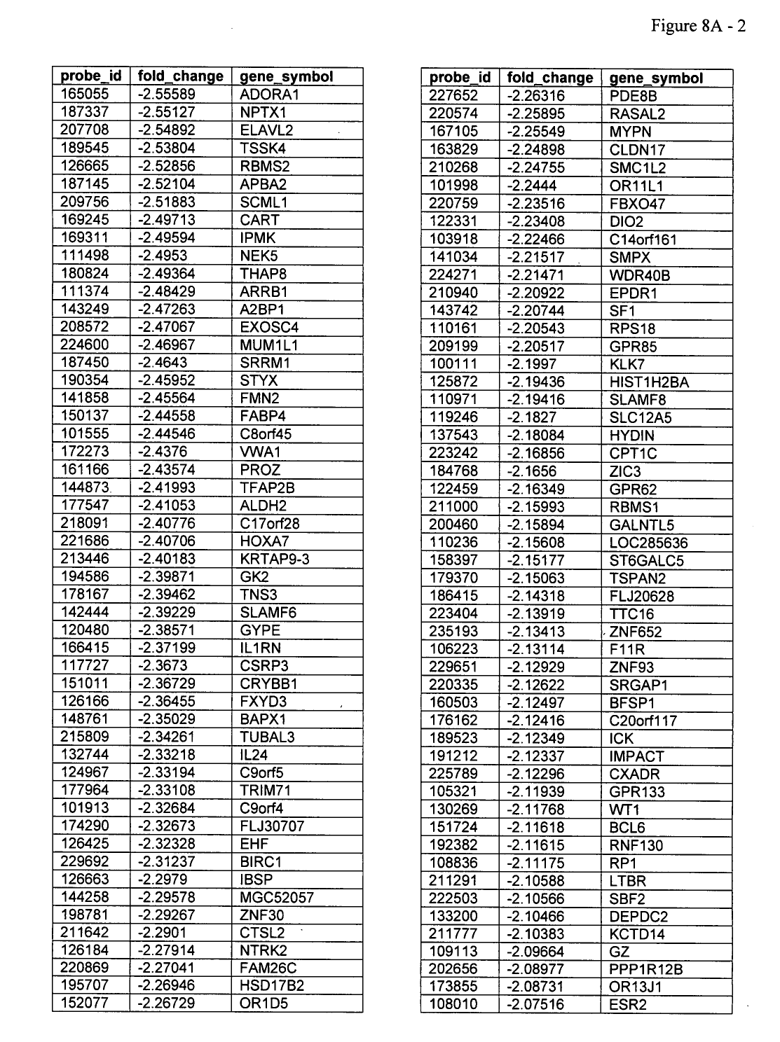

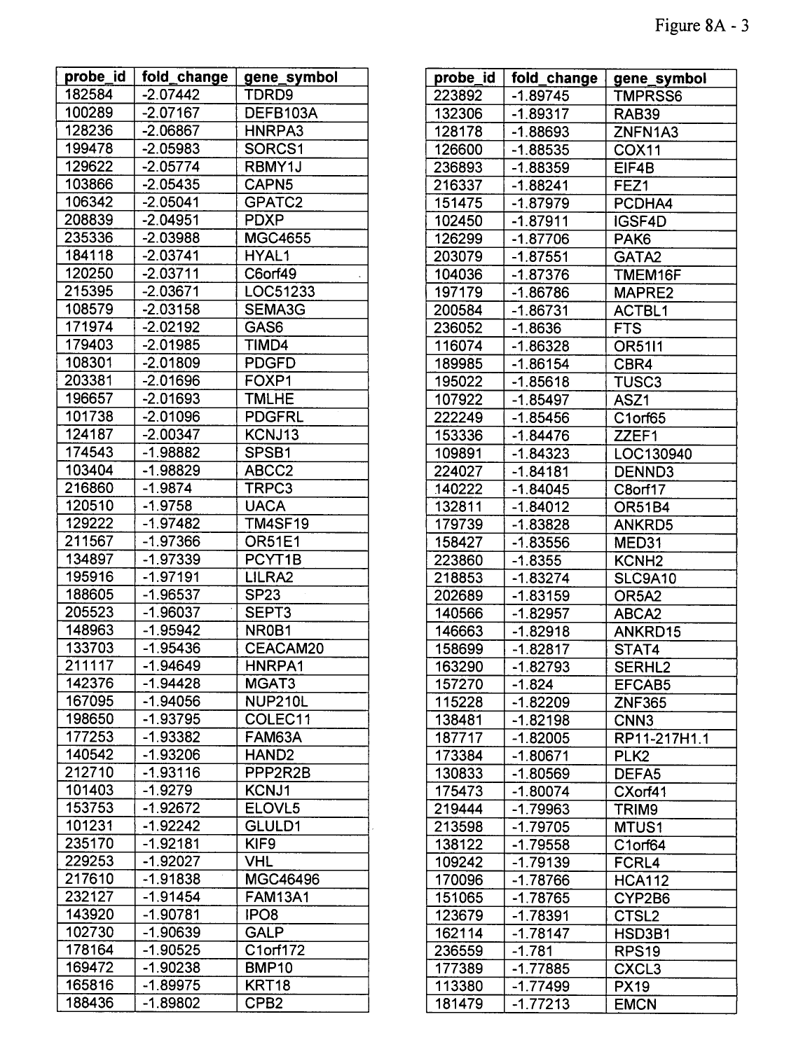

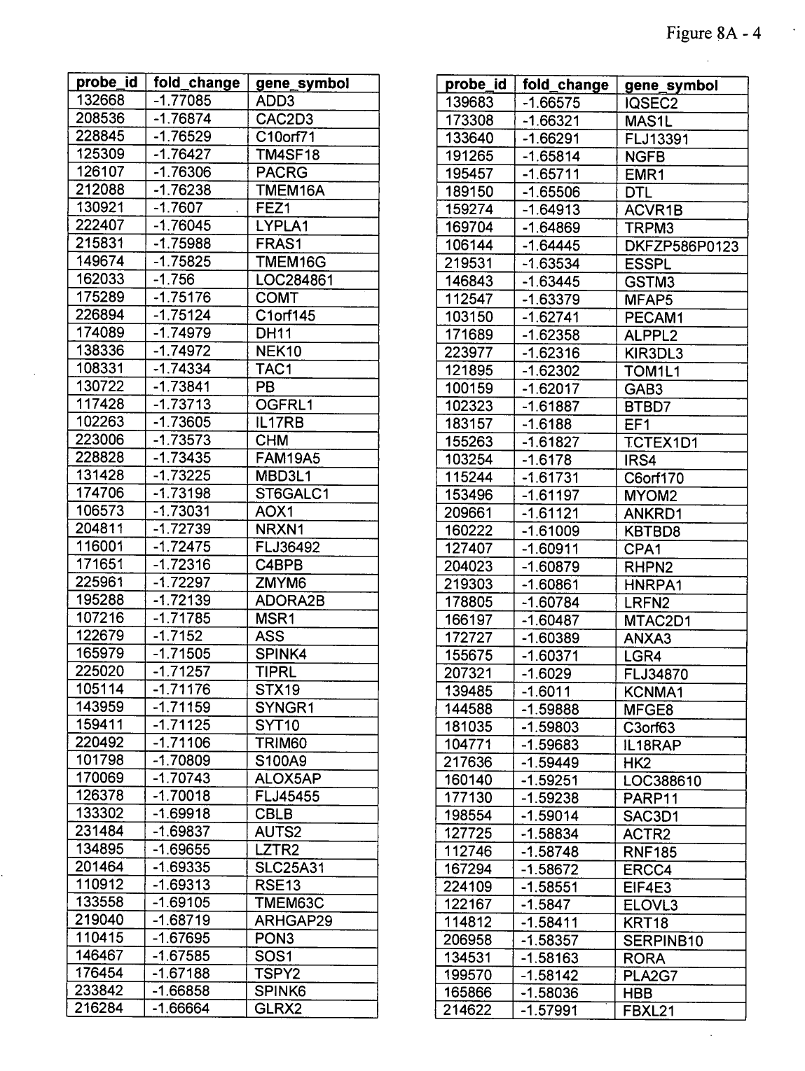

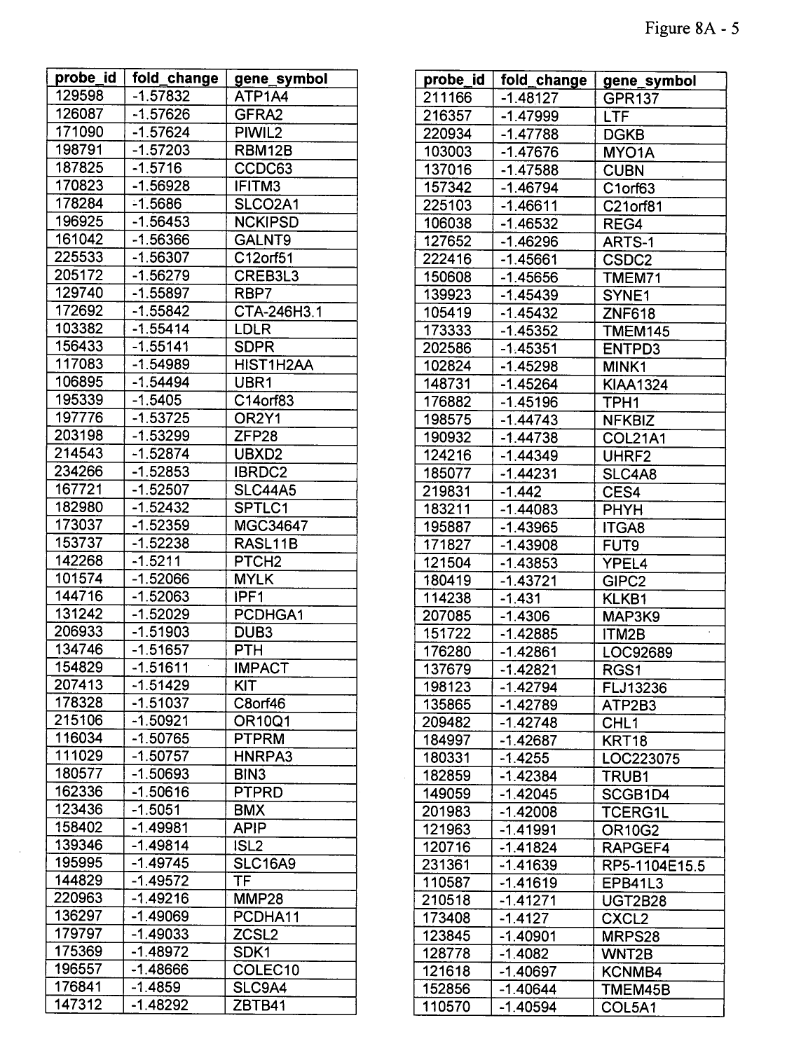

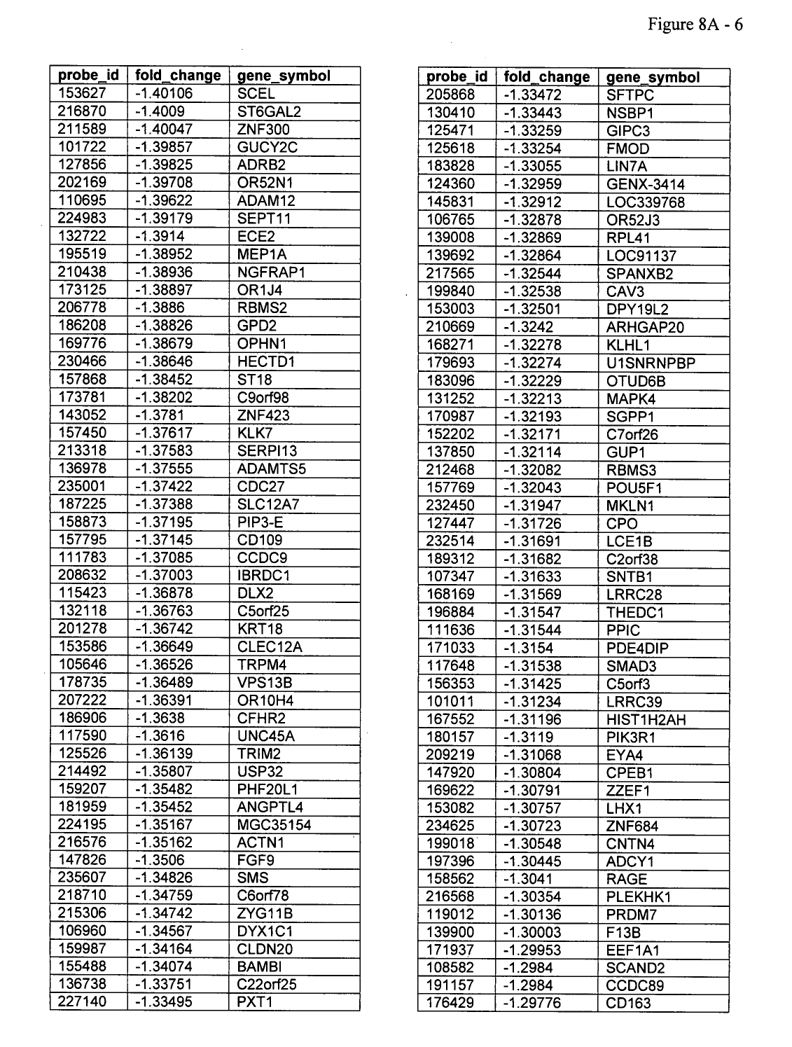

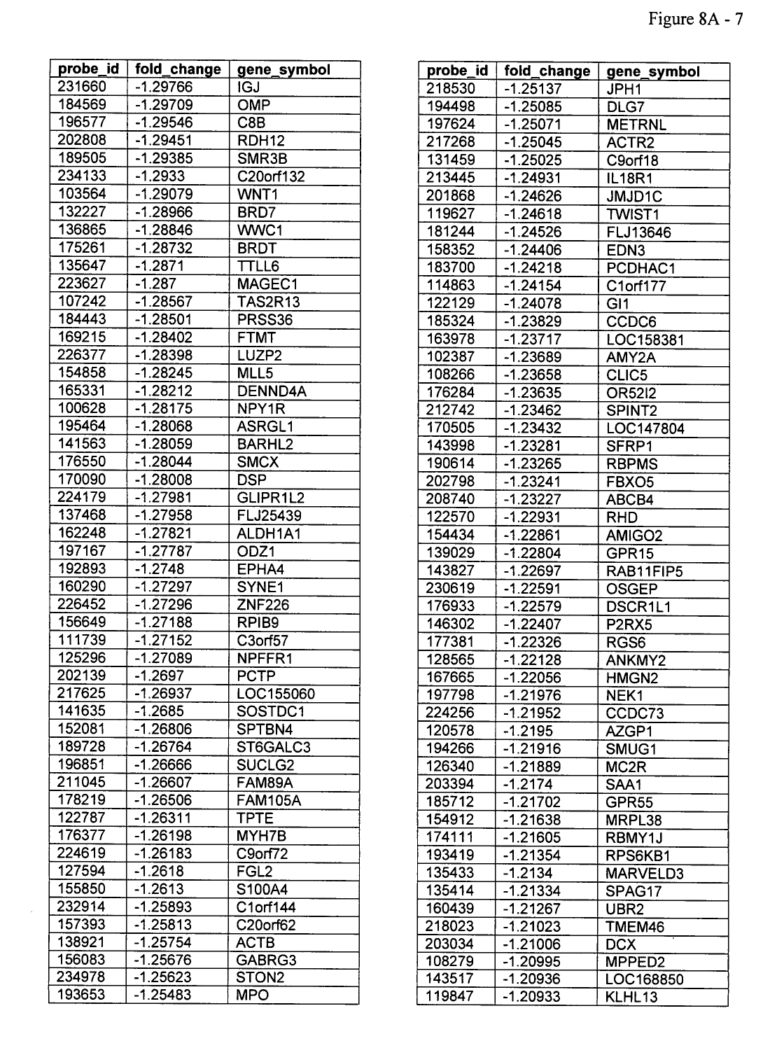

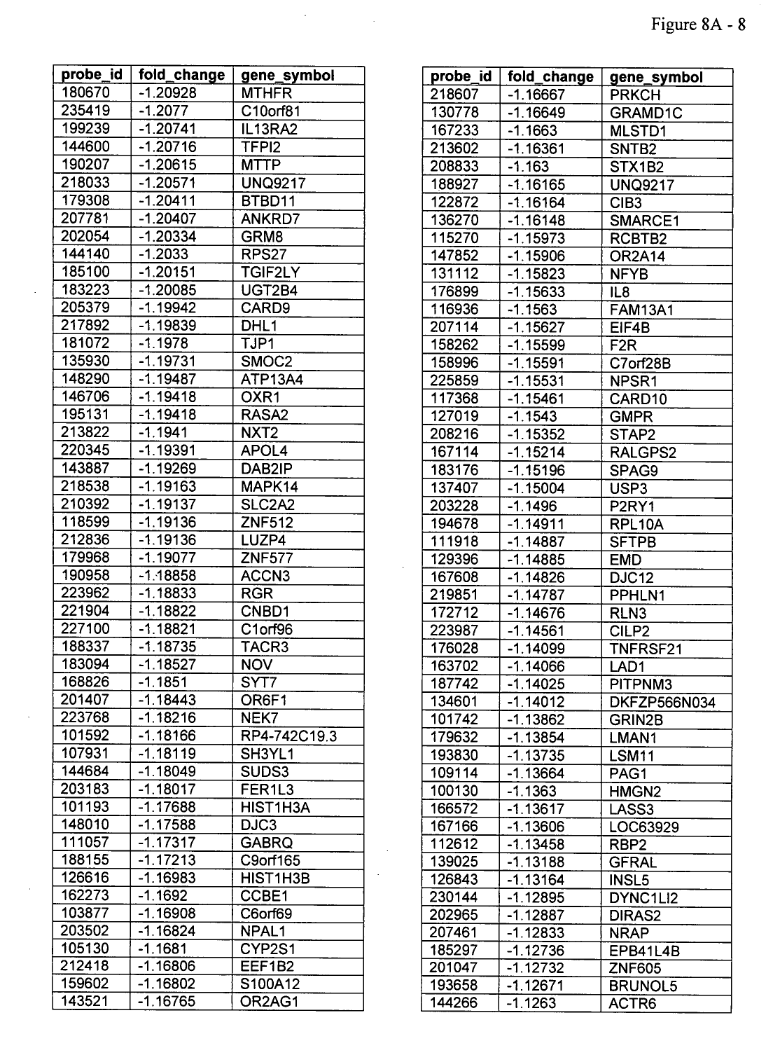

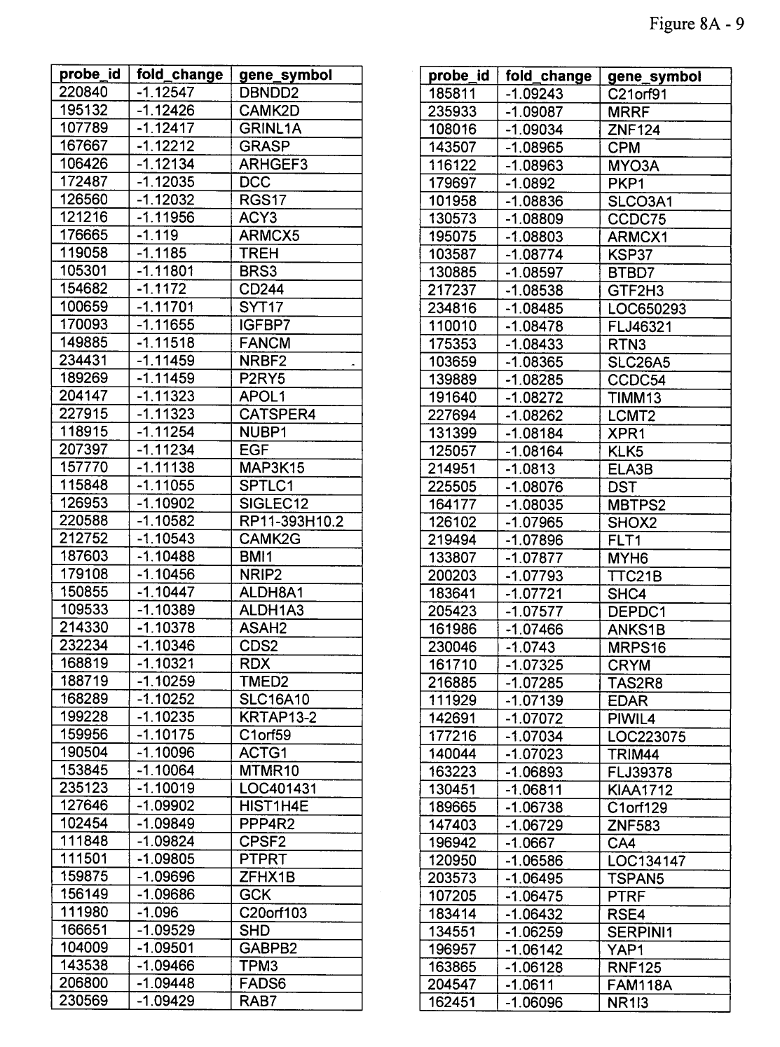

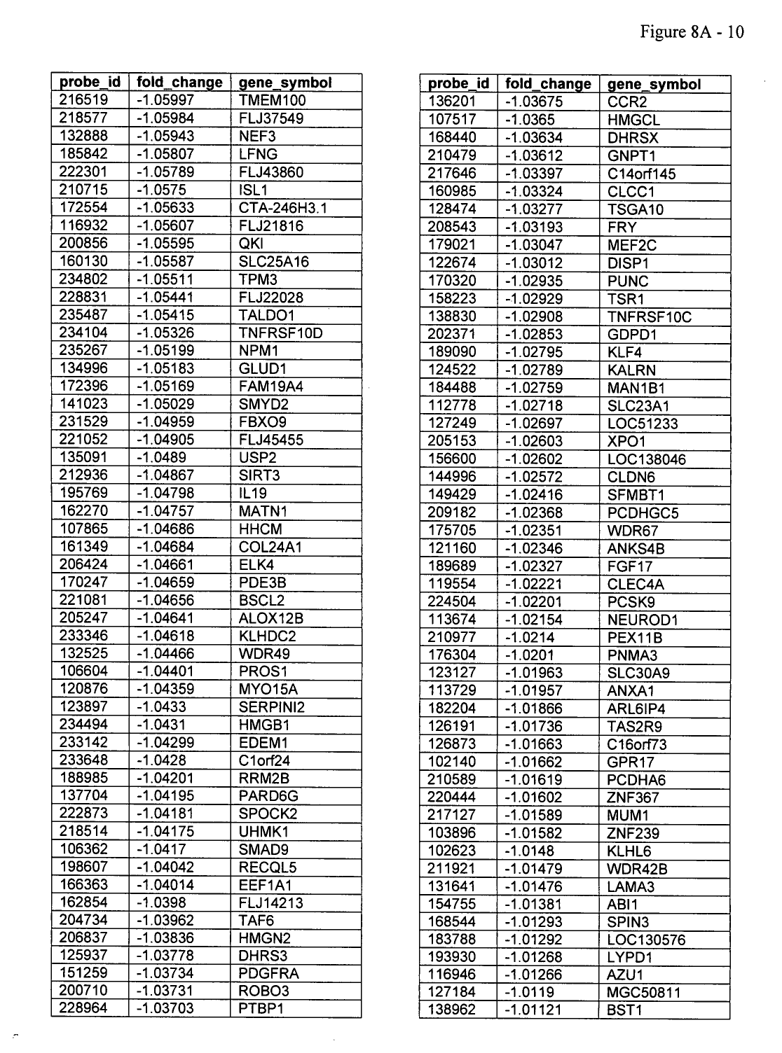

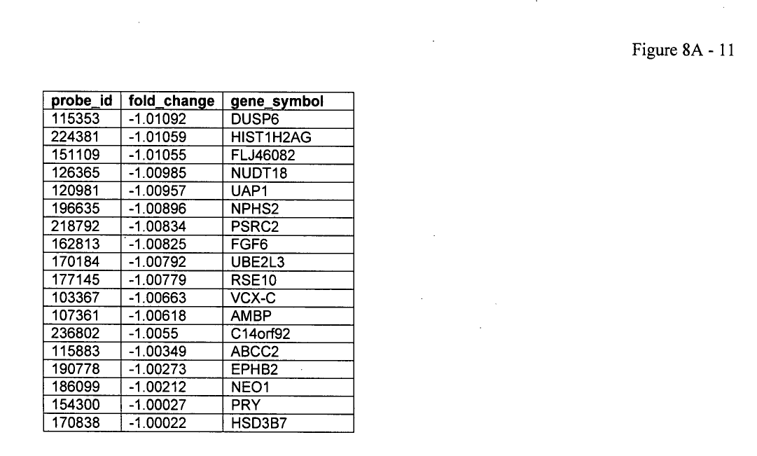

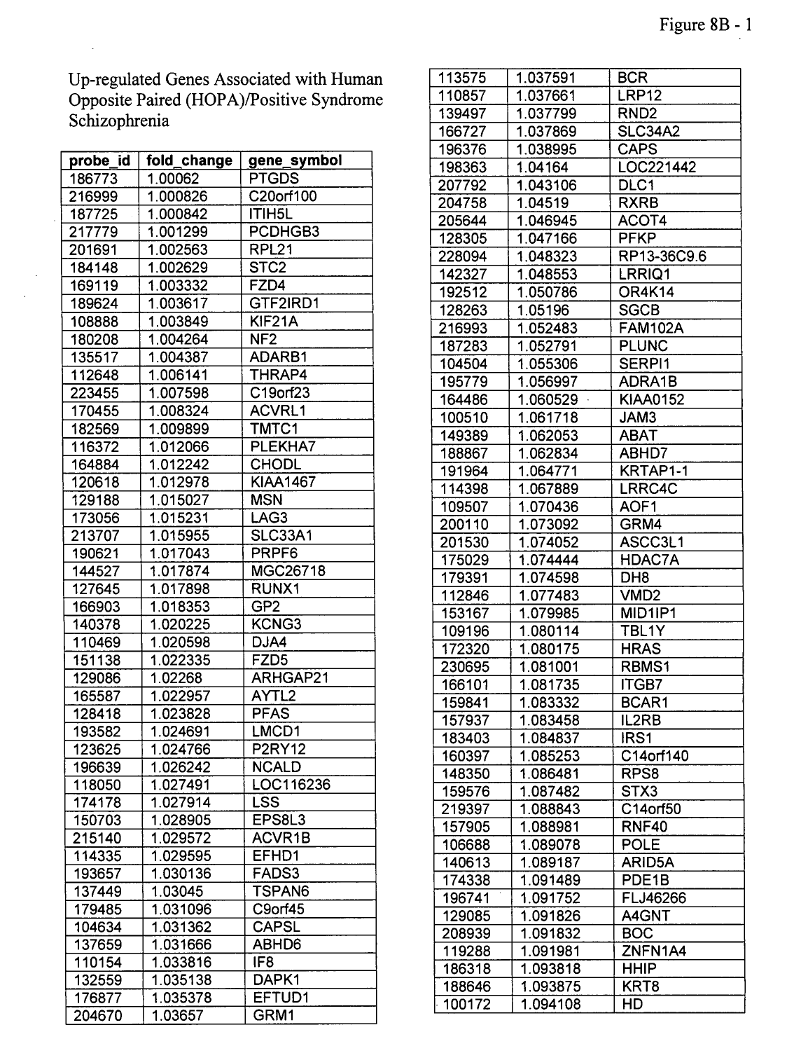

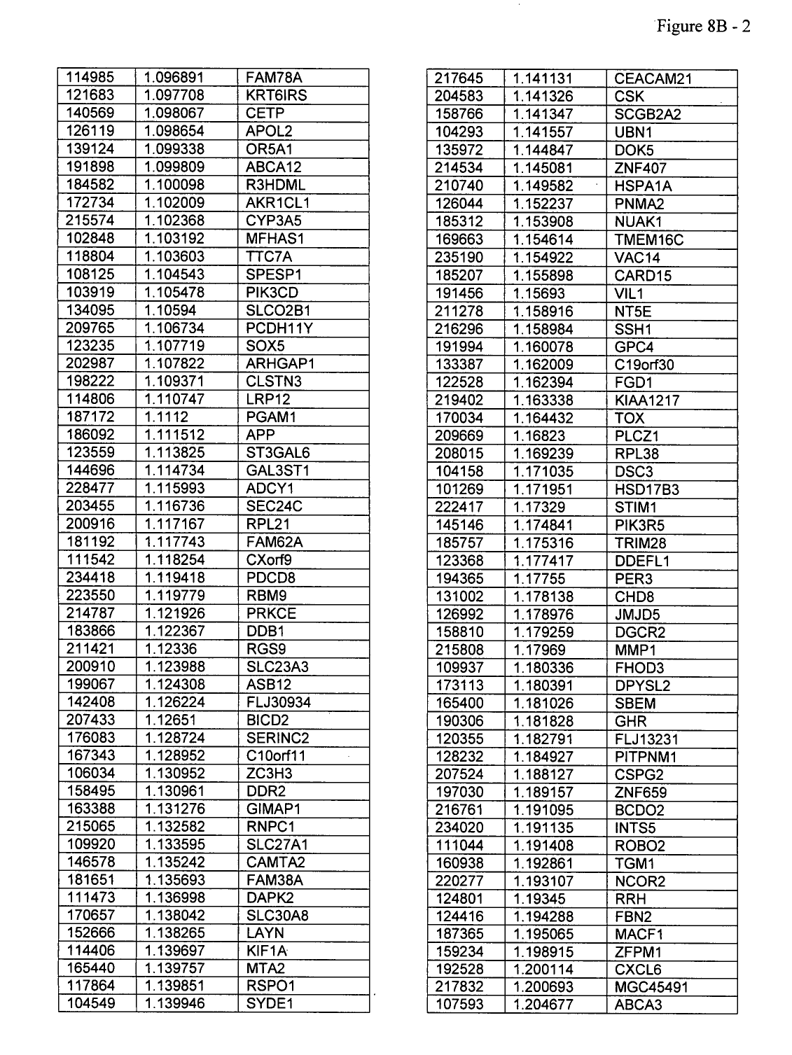

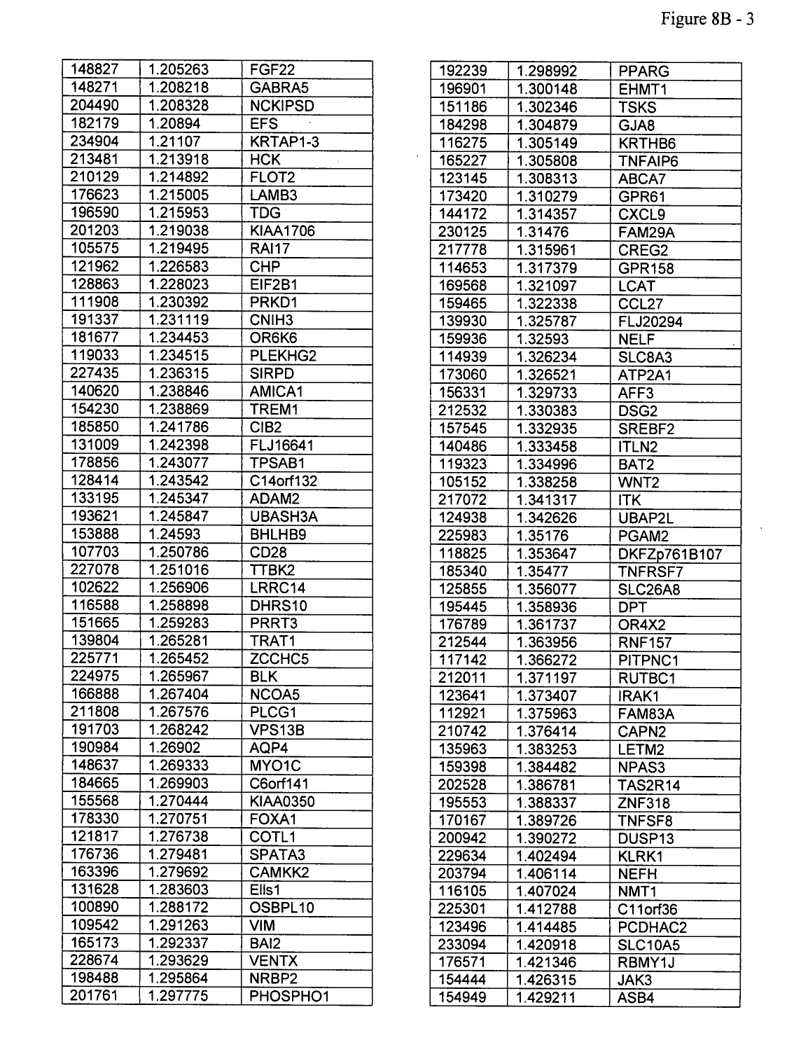

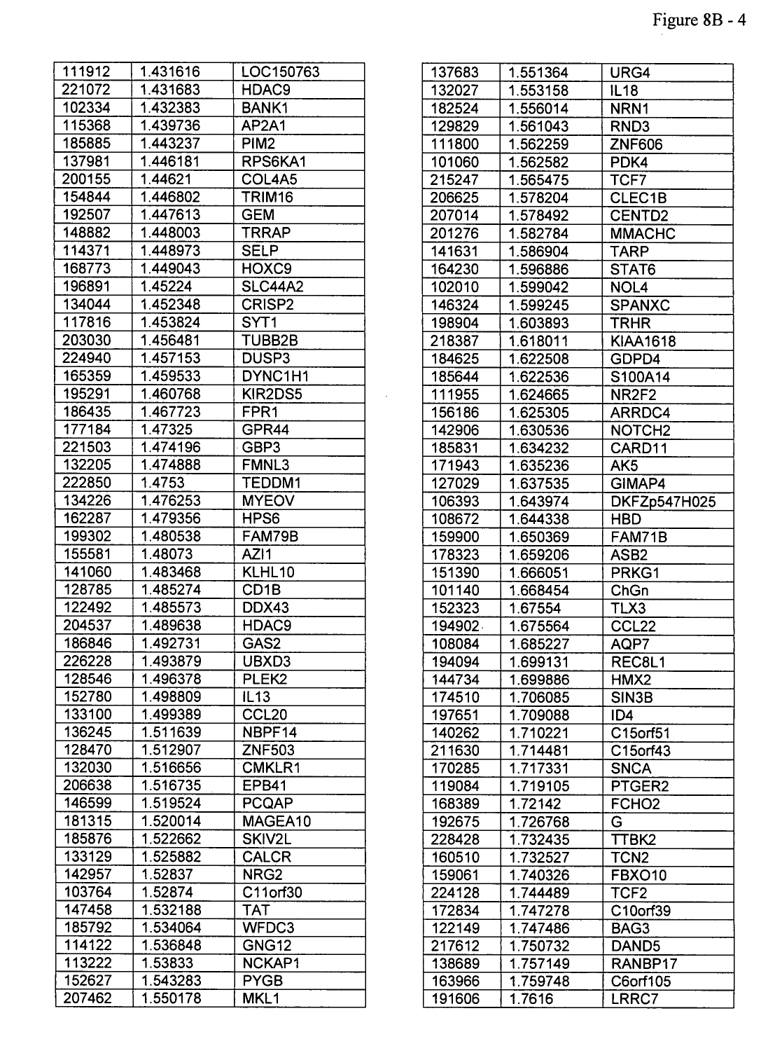

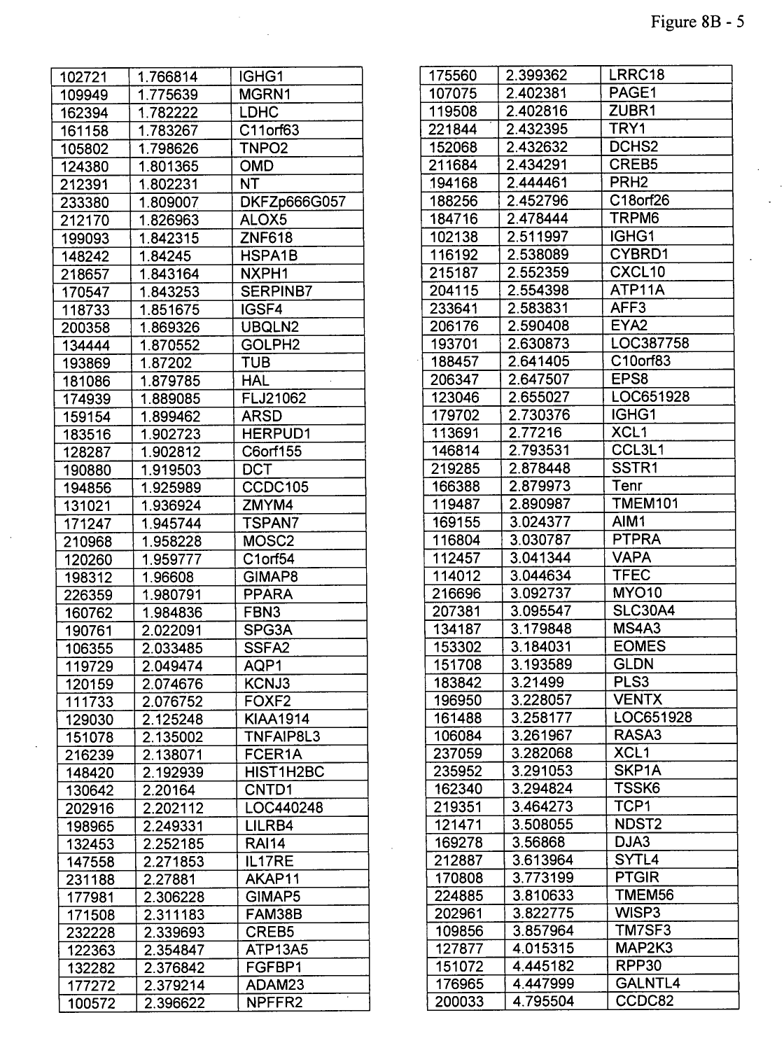

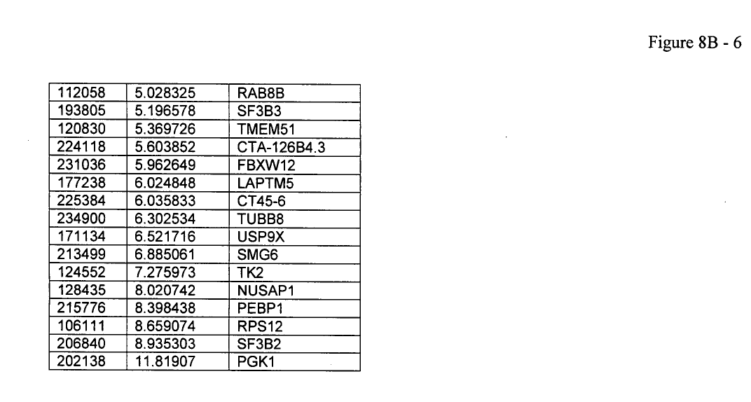

Because quantification using hybridization based microarray assays is sometimes unreliable, we tested the expression levels of five of the differentially expressed transcripts that had strong levels of expression in hemopoetic tissues (see the world-wide-web at genecards.org) using RT-PCR. TCP1, GYS2, DERL3 and KIAA 1183 were chosen from the list containing the transcripts up-regulated in all subjects. In addition, we choose to test the expression of COMT, which was only significantly up-regulated in males, because of its obvious relevance to biological psychiatry. FIGS. 2 and 3 show the results of that analysis. Consistent with overall data, TCP1, DERL3 and KIAA1183 levels were significantly increased in PD cases versus well controls (p<0.001, p<0.05, and p<0.008) after adjustment for GAPDH and LDHA levels. In contrast, neither GYS2 nor COMT expression was significantly associated with affected status. Interestingly, those three transcripts with the highest level of expression using RTPCR, TCP1 , DERL3 and KIAA1183, were successfully validated. Those genes with the lowest levels of expression using RTPCR, GYS2 and COMT, were not validated.

Next, a functional cluster analysis was conducted using the Panther pathway analysis program (see world-wide-web at pantherdb.org). No significant differences were observed in any functional gene cluster.

### DISCUSSION

In summary, the inventors report significant differences in the transcriptional profiles of lymphoblast cell lines derived from subjects with panic disorder versus those derived from psychiatrically well controls. However, before discussing these results a few caveats should be acknowledged. First, the RNA profiled in this study was derived from cultured lymphoblast cell lines, not primary CNS material. Second, it is possible that some of the changes noted, could be secondary to the presence of psychiatric medications, though two of the PD subjects were not on any medication. Third, the male PD lymphoblast lines were derived from younger subjects than their matched PD controls. Fourth, the lymphoblast lines from the PD subjects are second passage lines (stored previously in liquid nitrogen) while the control lines are newly created.

Despite the caveats, these observations are intriguing. The analysis of the transcriptional profiles was structured so that only those transcripts that were consistently up-regulated or down-regulated in every cell line were reported. There are a number of other transcripts whose expression is significantly changed overall as compared to the matched controls, but not arithmetically changed in the same direction in every cell line. However, given that this is a new area of investigation and the relevance of gene expression in these cells as compared to that in the relevant areas of the CNS is completely unknown, the inventors preferred to err on the conservative side. Nevertheless, the finding that so many genes in these cell lines are consistently and differentially expressed is remarkable.

In general, due caution must be utilized when reviewing microarray data. Microarray quantification is based on hybridization which, though sensitive, may be prone to false positives when the transcript copy number is low. The strength of the correlation of the RT-PCR and microarray signals is improved by using probe sets that recognize the same transcript. The current study took advantage of this by using microarrays which are paired to specific primer probe sets. Still, even when using complementary probe sets, the correlation between RT-PCR and microarray fold quantification is less than 0.9.

The expression of some of the transcripts quantified by the microarrays cannot be easily verified using RT-PCR. This may be in part because each microarray analysis uses more RNA (2 μg) than the normal RT-PCR reaction (12.5 ng in this study). For example, despite the fact that microarrays could measure 5HT3B and 5HT4 receptors, which were modestly up-regulated, the inventors could not detect them in these samples using RT-PCR. Conceivably, one could add more cDNA to the reaction tubes, but the kinetics of the PCR reaction can be adversely affected by excessive amounts of cDNA rendering the validation less useful.

In summary, we report that the transcriptional signatures of lymphoblast cell lines derived from PD subjects are significantly different from that of those of psychiatrically well subjects.

### EXAMPLE 3

### Antisocial Personality Disorder (ASPD)

The probe list for Antisocial Personality Disorder was derived using the same approach as delineated in our recent publications in the American Journal of Medical Genetics whose content is described in Examples 1 and 2. Briefly, lymphoblast RNA derived from 17 subjects and 14 controls was prepared as previously described and the resulting material was hybridized to ABI 1700 Human Genome Expression Micro-Array containing 29,098 gene specific oligonucleotide probes per the manufacturer's specifications. Storey q value method and the Spotfire™ analytic program were then used to assess false discovery rate to help determine the number of genes differentially expressed in the cell lines (Storey et al., Methods Mol Biol 2003; 224:149-57). The significantly up-regulated and down-regulated gene lists (7A and 7B) represent those genes whose expression was significantly changed in the cases as compared to controls.

### EXAMPLE 4

### Major Depression Disorder

The probe list for Major Depressive Disorder was derived using the same approach as delineated in our recent publications in the American Journal of Medical Genetics whose content is described in Examples 1 and 2. Briefly, lymphoblast RNA was prepared as previously described and the resulting material was hybridized to ABI 1700 Human Genome Expression Micro-Array containing 29,098 gene specific oligonucleotide probes per the manufacturer's specifications. Storey q value method and the Spotfire™ analytic program were then used to assess false discovery rate to determine the number of genes differentially expressed in the cell lines (Storey et al., Methods Mol Biol 2003; 224:149-57). The significantly up-regulated and down-regulated gene lists (8A and 8B) represent those genes whose expression was significantly changed in 8 of 10 of the cases as compared to 10 of 12 controls.

### EXAMPLE 5

### Positive Symptom Schizophrenia Such as that Associated with the HOPA^{12bp} Allele

The probe list for HOPA Syndrome Positive Symptom Psychosis was derived using the same approach as delineated in our recent publications in the American Journal of Medical Genetics whose content is described in Examples 1 and 2. Briefly, lymphoblast RNA derived from five psychotic subjects with the HOPA12bp polymorphism and five controls was prepared as previously described and the resulting material was hybridized to ABI 1700 Human Genome Expression Micro-Array containing 29,098 gene specific oligonucleotide probes per the manufacturer's specifications. Storey q value method and the Spotfire™ analytic program were then used to assess false discovery rate to help determine the number of genes differentially expressed in the cell lines (Storey et al., Methods Mol Biol 2003; 224:149-57). The significantly up-regulated and down-regulated gene lists (9A and 9B) represent those genes whose expression was significantly changed in the cases as compared to controls.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A screening kit for determining whether a subject has a predisposition to, or likelihood of having, a substance use disorder, or mental illness or syndrome comprising:
(a) a solid substrate, at least one probe specific for a down-regulated gene associated with nicotine dependence, and/or at least one probe specific for an up-regulated gene associated with nicotine dependence, wherein each probe is bound onto the substrate in a distinct spot; and/or
(b) a solid substrate, at least one probes specific for a down-regulated gene associated with panic disorder, and/or at least one probe specific for an up-regulated gene associated with panic disorder, wherein each probe is bound onto the substrate in a distinct spot; and/or
(c) a solid substrate, at least one probe specific for a down-regulated gene associated with Antisocial Personality Disorder (ASPD), and/or at least one probe specific for an up-regulated gene associated ASPD, wherein each probe is bound on the substrate in a distinct spot; and/or
(d) a solid substrate, at least one probe specific for a down-regulated gene associated with Major Depressive Disorder (MDD), and/or at least one probe specific for an up-regulated gene associated with MDD, wherein each probe is bound onto the substrate in a distinct spot; and/or
(e) a solid substrate, at least one probe specific for a down-regulated gene associated with positive symptom schizophrenia such as that associated with the HOPA^{12bp} allele, and/or at least one probe specific for an up-regulated gene associated with positive symptom schizophrenia such as that associated with the HOPA^{12bp} allele, wherein each probe is bound onto the substrate in a distinct spot.

2. The kit of claim 1, wherein the substrate is a polymer, glass, semiconductor, paper, metal, gel or hydrogel.

3. The kit of claim 1, further comprising a solid substrate and at least one control probe, wherein the at least one control probe is bound onto the substrate in a distinct spot.

4. The kit of claim 1, wherein the solid substrate is a microarray.

5. The kit of claim 1, wherein the probe is an oligonucleotide probe.

6. The kit of claim 1, wherein the probe is a nucleic acid derivative probe.

7. A screening kit for determining whether a subject has a predisposition to, or likelihood of having, a substance use disorder, or mental illness or syndrome comprising:
(a) a PCR or RTPCR assay comprising at least one primer probe set specific for a down-regulated gene associated with nicotine dependence, and/or at least one primer probe set specific for an up-regulated gene associated with nicotine dependence, and/or
(b) a PCR or RTPCR assay comprising at least one primer probe set specific for a down-regulated gene associated with panic disorder, and/or at least one primer probe set specific for an up-regulated gene associated with panic disorder, and/or
(c) a PCR or RTPCR assay comprising at least one primer probe set specific for a down-regulated gene associated with Antisocial Personality Disorder (ASPD), and/or at least one primer probe set specific for an up-regulated gene associated ASPD, and/or
(d) a PCR or RTPCR assay comprising at least one primer probe set specific for a down-regulated gene associated with Major Depressive Disorder (MDD), and/or at least one primer probe set specific for an up-regulated gene associated with MDD; and/or
(e) a PCR or RTPCR assay, at least one primer probe set specific for a down-regulated gene associated with positive symptom schizophrenia such as that associated with the HOPA^{12bp} allele, and/or at least one primer probe set specific for an up-regulated gene associated with positive symptom schizophrenia such as that associated with the HOPA^{12bp} allele.

8. The kit of claim 7, wherein the probe is an oligonucleotide probe.

9. The kit of claim 7, wherein the probe is a nucleic acid derivative probe.

10. A diagnostic method for determining whether a subject has a predisposition to, or likelihood of having, a substance use disorder or mental illness or syndrome, by determining a nucleic acid expression profile from a single type of blood cell or blood cell derivative from the subject, the method comprising:
(a) obtaining a profile associated with the sample, wherein the profile comprises quantitative data for at least one down-regulated gene associated with nicotine dependence, at least one up-regulated gene associated with nicotine dependence, at least one down-regulated gene associated with panic disorder, at least one up-regulated gene associated with panic disorder, at least one down-regulated gene associated with ASPD, at least one up-regulated gene associated with ASPD, at least one down-regulated gene associated with MDD, at least one up-regulated gene associated with MDD, at least one down-regulated gene associated with positive symptom schizophrenia such as that associated with the HOPA^{12bp} allele, or at least one up-regulated gene associated with positive symptom schizophrenia such as that associated with the HOPA^{12bp} allele;
(b) inputting the data into an analytical process that uses the data to classify the sample, wherein the classification is a “substance use disorder or mental illness or syndrome” classification or a “healthy” classification; and
(c) classifying the sample according to the output of the process.

11. The method of claim 10, wherein the analytical process comprises comparing the obtained profile with a reference profile.

12. The method of claim 11, wherein the reference profile comprises data obtained from one or more healthy control subjects, or comprises data obtained from one or more subjects diagnosed with a substance use disorder or mental illness or syndrome.

13. The method of claim 11, further comprising obtaining a statistical measure of a similarity of the obtained profile to the reference profile.

14. The method of claim 10, wherein the blood cell is a lymphocyte.

15. The method of claim 14, wherein the lymphocyte type is a B-lymphocyte.

16. The method of claim 15, wherein the B-lymphocytes have been immortalized.

17. The method of claim 10, wherein the blood cell type is a monocyte.

18. The method of claim 10, wherein the blood cells type is a basophil.

19. The method of claim 10, wherein the mental illness or syndrome is major depression disorder, antisocial personality disorder, panic disorder, bipolar disorder or schizophrenia.

20. The method of claim 10, wherein the substance use disorder is nicotine dependence, alcohol dependence or cannabis dependence.

21. A composition for determining whether a subject has a predisposition to, or likelihood of having nicotine dependence comprising:
(a) a solid substrate; and
(b) at least one down-regulated probe specific for a down-regulated gene associated with nicotine dependence, wherein each down-regulated probe is bound onto the substrate in a distinct spot, and/or at least one up-regulated probe specific for an up-regulated genes associated with nicotine dependence, wherein each up-regulated probe is bound onto the substrate in a distinct spot.

22. A composition for determining whether a subject has a predisposition to, or likelihood of having panic disorder comprising:
(a) a solid substrate; and
(b) at least one down-regulated probe specific for a down-regulated gene associated with panic disorder, wherein each down-regulated probe is bound onto the substrate in a distinct spot, and/or at least one up-regulated probe specific for an up-regulated gene associated with panic disorder, wherein each up-regulated probe is bound onto the substrate in a distinct spot.

23. A composition for determining whether a subject has a predisposition to, or likelihood of having Antisocial Personality Disorder (ASPD) comprising:
(a) a solid substrate; and
(b) at least one down-regulated probe specific for a down-regulated gene associated with ASPD, wherein each down-regulated probe is bound onto the substrate in a distinct spot, and/or at least one up-regulated probe specific for an up-regulated gene associated with ASPD, wherein each up-regulated probe is bound onto the substrate in a distinct spot.

24. A composition for determining whether a subject has a predisposition to, or likelihood of having Major Depressive Disorder (MDD) comprising:
(a) a solid substrate; and
(b) at least one down-regulated probe specific for a down-regulated gene associated with MDD, wherein each down-regulated probe is bound onto the substrate in a distinct spot, and/or at least one up-regulated probe specific for an up-regulated gene associated with MDD, wherein each up-regulated probe is bound onto the substrate in a distinct spot.

25. A composition for determining whether a subject has a predisposition to, or likelihood of having positive symptom schizophrenia such as that associated with the HOPA^{12bp} allele comprising:
(a) a solid substrate; and
(b) at least one down-regulated probe specific for a down-regulated gene associated with positive symptom schizophrenia such as that associated with the HOPA^{12bp} allele, wherein each down-regulated probe is bound onto the substrate in a distinct spot, and/or at least one up-regulated probe specific for an up-regulated gene associated with associated with positive symptom schizophrenia such as that associated with the HOPA^{12bp} allele, wherein each up-regulated probe is bound onto the substrate in a distinct spot.

26. A composition for determining whether a subject has a predisposition to, or likelihood of having nicotine dependence comprising a PCR or RTPCR assay kit comprising at least one primer-probe set specific for a down-regulated gene associated with nicotine dependence, and/or at least one primer-probe set specific for an up-regulated gene associated with nicotine dependence.

27. A composition for determining whether a subject has a predisposition to, or likelihood of having panic disorder comprising a PCR or RTPCR assay kit comprising at least one primer-probe set specific for a down-regulated gene associated with panic disorder, and/or at least one primer-probe set specific for an up-regulated gene associated with panic disorder.

28. A composition for determining whether a subject has a predisposition to, or likelihood of having Antisocial Personality Disorder (ASPD) comprising a PCR or RTPCR assay kit comprising at least one primer-probe set specific for a down-regulated gene associated with ASPD, and/or at least one primer-probe set specific for an up-regulated gene associated with ASPD.

29. A composition for determining whether a subject has a predisposition to, or likelihood of having Major Depressive Disorder (MDD) comprising a PCR or RTPCR assay kit comprising at least one primer-probe set specific for a down-regulated gene associated with MDD, and/or at least one primer-probe set specific for an up-regulated gene associated with MDD.

30. A composition for determining whether a subject has a predisposition to, or likelihood of having positive symptom schizophrenia such as that associated with the HOPA^{12bp} allele comprising a PCR or RTPCR assay kit comprising at least one primer-probe set specific for a down-regulated gene associated with positive symptom schizophrenia such as that associated with the HOPA^{12bp} allele, and/or at least one primer-probe set specific for an up-regulated gene associated with positive symptom schizophrenia such as that associated with the HOPA^{12bp} allele.
